(19) United States
(12) Patent Application Publication
(10) Pub. No.: US 2004/0235792 A1
(43) Pub. Date: Nov. 25, 2004

(54) Serine protease inhibitors compromising a hydrogen-bond acceptor

(76) Inventors: John J Deadman, London (GB); John Spencer, London (GB); Paulette A Greenidge, Oberwil (CH); Christopher A Goodwin, London (GB); Vijay Vir Kakkar, London (GB); Michael F Scully, London (GB); Richard Jones Holland, London (GB)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 10/466,736

(22) PCT Filed: Jan. 18, 2002
(86) PCT No.: PCT/GB02/00224

Related U.S. Application Data
(60) Provisional application No. 60/267,172, filed on Feb. 6, 2001.

(30) Foreign Application Priority Data
Jan. 20, 2001 (GB) 0101537.9

(51) Int. Cl. A61K 31/69; C07F 5/04

(52) U.S. Cl. 514/64; 558/287

(57) ABSTRACT

Compounds, useful as protease inhibitors, of the formula (I): where: Ar is a ring or ring system, for example a benzene ring, and may be substituted by one or more moieties in addition to X and LJ; X is a functional group which is a hydrogen-bond acceptor, e.g. a nitro or boronate group BY¹Y²; L is a linker, most preferably (CR⁵R⁶)—S—; J is a moiety containing a basic nitrogen atom but not containing an amino acid residue, preferably amidino, guanidine, amino carboxamido, hydroxylamino, or imidazolyl, or an N-substituted analogue thereof.

### BACKGROUND OF THE INVENTION

The present invention relates to non-peptide compounds which are useful in particular, but not exclusively, as inhibitors of proteases and other enzymes.

It is convenient to commence describing the detailed background to the invention by providing a glossary of certain technical terms and abbreviations:

α-Aminoboronic acid or Boro(aa) refers to an amino acid in which the CO₂ group has been replaced by BO₂

Boc—tertiarybutyloxycarbonyl

CG—cathepsin G

Chy—chymotrypsin

Dibal—diisobutyl aluminium hydride.

Ela—elastase

FVIIa-TF—factor VIIa-tissue factor complex

Kal—kallikrein

Pca—protein C activated

Pinacol—2,3-dimethyl-2,3-butanediol

(+)-Pinanediol boronate—1a,7,7-trimethyl-[1aS-{1aa,4a,6a,5aa}]4,6-methano-1,2-benzodioxaborole

Pla—plasmin

Plg—plasminogen

TBTU—2-(1H-Benzotriazole-1-yl)-1,1,3,3-tetramethyluronium tetrafluoroborate

TFA—trifluoroacetic acid

Thr—thrombin

t-PA—tissue plasminogen activator

Try—trypsin

IXa—factor IX activated

Xa—factor X activated

UK—urokinase

Initial encounter complex [EI] (also known as the Michaelis complex) refers to an interaction charcterised by the equation:

E+I=EI→EI*, where E is the enzyme, I the inhibitor and EI* the final complex after slow-tight binding.³ Initial Ki refers to the formation of EI and final Ki (Ki*) to EI*.

P1, P2, P3, etc. designate substrate or inhibitor residues which are amino-terminal to the scissile peptide bond, and S1, S2, S3, etc., designate the corresponding subsites of the cognate proteinase in accordance with: Schechter, I. and Berger, A. On the Size of the Active Site in Proteases, Biochem. Biophys. Res. Comm., 1967, 27, 157-162.

Proteases are enzymes which cleave proteins at specific peptide bonds. Cuypers et al., J. Biol. Chem. 257:7086 (1982), and the references cited therein, classify proteases on a mechanistic basis into five classes: serine, cysteinyl or thiol, acid or aspartyl, threonine and metalloproteases. Members of each class catalyse the hydrolysis of peptide bonds by a similar mechanism, have similar active site amino acid residues and are susceptible to class-specific inhibitors. For example, all serine proteases that have been characterised have an active site serine residue, and are susceptible to inhibition by boronic adds and their derivatives.

For example, Koehler et al. Biochemistry 10: 2477 (1971) report that 2-phenylethane boronic add inhibits chymotrypsin at millimolar levels. The inhibition of chymotrypsin and subtilisin by arylboronic acids (phenylboronic acid, m-nitro-phenylboronic acid, m-aminophenylboronic acid, m-bromophenylboronic acid) is reported by Phillip et al, Proc. Nat Acad. Sci. USA 68: 478-480 (1971). A study of the inhibition of subtilisin Carlsberg by a variety of boronic acids, especially phenyl boronic acids substituted by Cl, Br, CH₃, H₂N, MeO and others, is described by Seufer-Wasserthal et al, Biorg. Med. Chem. 2(1): 35-48 (1994).

Pharmaceutical research has moved from the simple arylboronic acids to boropeptides, i.e. peptides containing a boronic acid analogue of an N-acyl-α-amino add. Shenvi (EP-A-145441) disclosed that peptides containing an α-aminoboronic add with a neutral side chain were effective inhibitors of elastase and has been followed by numerous patent publications relating to boropeptide inhibitors of serine proteases. Specific, tight binding boronic acid inhibitors have been reported for elastase (Kᵢ, 0.25 nM), chymotrypsin (Kᵢ, 0.16 nM), cathepsin G (Kᵢ, 21 nM), α-lytic proteinase (Kᵢ, 0.25 nM), dipeptidyl aminopeptidase type IV (Kᵢ, 16 pM) and more recently thrombin (Ac-D-Phe-Pro-boro Arg-OH (DuP 714 initial Kᵢ 1.2 nM).

More recently, Amparo et al (WO 96/20689) have disclosed boropeptide thrombin inhibitors containing an aryl or heteroaryl group linked to the boronate residue or derivative through a linker featuring the structure:

-Z-C(H)(R²)—,

where Z is —(CH₂)ₘCONR⁸—, —(CH₂)ₘCSNR⁸—, —(CH₂)ₘSO₂NR⁸—, —(CH₂)ₘCO₂—, —(CH₂)ₘC(S)O—, or —(CH₂)ₘSO₂O—, and R² is a side chain substituted with halogen, nitrile, —NO₂, —CF₃, S(O)ᵣR¹⁴ or certain basic groups. Typically, the linker includes an arginine, lysine or thioarginine residue. These compounds contain a boroamino acid residue [>N—C(H)(R²)—B<] or an analogue in which the α-nitrogen has been replaced by an oxygen.

The scientific literature has featured a number of publications relating to thrombin inhibition, a selection of which it is useful to review:

Thrombin Inhibition and Thrombin Inhibitors: Background In contrast to heparin and hirudin (Markwardt, F. Hirudin: The Promising Antithrombotic. Cardiovascular Drug Reviews. 1992, 10, 211-232.), synthetic low molecular weight inhibitors show more or less equivalent potency as regards the inhibition of thrombin in solution and within the thrombus (Weitz, J. I.; Hudoba, M.; Massel, D.; Maraganore, J. and Hirsh, J. Clot-bound Thrombin is Protected from inhibition by Heparin-antithrombin III but is Susceptible to Inactivation by Antithrombin III-independent Inhibitors. J. Clin. Invest 1990, 86, 385-391.) and there have now been two reports of the use of low molecular weight thrombin inhibitors as adjuncts to thrombolytic therapy (Shebuski, R. J. Principles underlying the use of Conjunctive Agents with Plasminogen Activators. Annals of the New York Academy of Sciences. 1992, 667, 382-394.; Klement, P.; Borm, A.; Hirsh, J.; Maraganore, J.; Wilson, G.; Weitz, J.; The Effect of Thrombin Inhibitors on Tissue Plasminogen Activator Induced Thrombolysis in a Rat Model. Thromb. Haemostasis. 1992, 68, 64-68.

Both H-D-MePhe-Pro-Arginal (LY294468, [Bajusz, S.; Szell, E.; Bagdy, D.; Barabas, E.; Horvath, G.; Dioszegi, M.; Fittler, Z.; Szabo, G.; Juhasz, A.; Tomori, E.; Szilagyi, G. Highly Active and Selective Anticoagulants: D-Phe-Pro-Arg-H, a Free Tripeptide Aldehyde Prone to Spontaneous inactivation, and its stable N-methyl derivative D-MePhe-Pro-Arg-H. J. Med. Chem. 1990, 33, 1729-1735 and references therein; Barabas, E.; Szell, E. and Bajusz, S. Screening for Fibrinolysis Inhibitory Effect of Synthetic Thrombin Inhibitors. Blood Coag. Fibrinol. 1993, 4, 243-248.] and Argatroban [Bush, L. R. Argatroban, A Selective Potent, Thrombin Inhibitor. Cardiovasc. Drug. Rev. 1991, 9, 247-263; Lyle, T. A. Small Molecule Inhibitors of Thrombin. Perspect. Drug. Dis. Des. 1993, 1, 453-460.] have been shown to be effective in reducing reocclusion rates in animal models of thrombolysis by t-PA; experimental conditions in which a heparin regimen was ineffective. [Jackson, C. V.; Wilson, H. C.; Growe, V. G.; Shuman, R. T. and Gesellchen, P. D.; Reversible Tripeptide Inhibitors as Adjunctive Agents In Coronary Thrombolysis: a Comparison with Heparin in a Canine Model of Coronary Artery Thrombosis. J. Cardiovasc. Pharmacol. 1993, 21, 587-594.].

Argatroban has also shown efficacy in patients undergoing thrombolytic therapy [Verstraete, M. and Zoldhelyi, P., Novel Antithrombotic Drugs in Development. Drugs, 1995, 49, 856-884.] but with rapid recurrence of angina, described as “rebound”, a phenomenon which, while not fully understood, may be partly due to inadequate anticoagulation. [Gold, H. K.; Torres, F. W.; Garabedian, H. D.; Werner, W.; Jang, I. K.; Khan, A.; Hagstrom, J. N.; Yasuda, T.; Leinbach, R. C.; Newell, J. B.; Bovill, E. G.; Stump, D. C. and Colien, D. Evidence for a Rebound Coagulation Phenomenon after Cessation of a 4 Hour Infusion of A Specific Thrombin Inhibitor in Patients with Unstable Angina Pectoris. J. Am. Coll. Cardiol., 1993, 21, 1039-1047; Granger, C. B.; Miller, J. M.; Bovill, E. G.; Gruber, A.; Tracy, R. P.; Krucoff, M. W.; Green, C.; Berrios, E.; Harrington, R. A.; Ohman, E. M. and Califf, R. Rebound Increase in Thrombin Generation and Activity After Cessation of Intravenous Heparin in Patients with Acute Coronary Syndromes, Circulation, 1995, 91, 1929-1935; Ofosu F A. Mechanisms for the Anticoagulant Effects of Synthetic Antithrombins. Adv. Exp. Med. Biol. 1993, 340, 213-226.

This deficiency may arise from the functional characteristics of Argatroban which acts as rapidly equilibrating competitive inhibitor of thrombin, a property which has been claimed to be adequate for anticoagulation if the inhibitory constant (Ki) is 10 nM or less [Powers, J. C. and Kam, C.-M. Synthetic Substrates and Inhibitors of Thrombin. in Thrombin, Structure and Function., Berliner, L.Ed.; Plenum Press, N.Y.1992; pp117-157; Stone S R and Tapparelli C. ‘Thrombin Inhibitors as Antithrombotic Agents: The Importance of Rapid Inhibition’. J. Enz. Inhib. 1995; 9, 3-15.]. However, within the context of inhibiting fibrin-bound thrombin, the properties of slow-tight binding inhibitors which can achieve much tighter binding (Kᵢ<50 pM) may be more efficient since the limited flow within the thrombus provides opportunity for equilibration with the target enzyme.

The compound, Ac-D-Phe-Pro-boro Arg (DuP 714) Kettner, C; Mersinger, L. and Knabb, R. The Selective Inhibition of Thrombin by Peptide of Boroarginine. J Biol Chem. 1990, 205, 18289-18297] is an excellent example of a highly potent and slow-tight binding inhibitor of thrombin (final K_{i*(Thr)} <10 pM). However, at the high concentrations required for effective anticoagulation it has been shown to compromise the activity of both the fibrinolytic system [Callas, D. D.; Bacher, P.; Iqbal, O.; Hoppensteadt, D.; Fareed, J. Fibrinolytic Compromise by Simultaneous Administration of Site-directed Inhibitors of Thrombin. Thrombosis Res. 1994, 74, 193-205.] and the protein Ca system (IC₅₀ 0.75 μM) [Callas, D. D. and Fareed, J. Direct Inhibition of Protein Ca by site directed Thrombin Inhibitors: Implications for Anticoagulant and Thrombolytic Therapy Thromb. Res., 1995, 78, 457-460.], limiting its use in adjunctive therapy.

Several groups have now recognised the significance of ensuring selectivity for thrombin, over the trypsin-like fibrinolytic enzymes in the development of antithrombotic agents for use in thrombolytic therapy. One strategy used to develop second generation analogues of the D-Phe-Pro-Arg based compounds, such as DuP 714, has been to enhance the favourable edge-on interaction of the aryl group with Trp-215 of the thrombin S3 site. Derivatization of the P2 Pro of dipeptide boronate based inhibitors for example with acyl groups, e.g. arylpropionyl or arylpyrrolidinyl [Fevig, J. M.; Abelman, M. M.; Brittelli, D. R.; Kettner, C. A.; Knabb, R. M. and Weber, P. C. ‘Design and Synthesis of Ring-constrained Boropeptide Thrombin Inhibitors’. Bioorg. Med. Chem. Lett 1996; 6, 295-300.] led to reduction in the antiparallel interaction with Gly-216, and loss of affinity for thrombin compared to the parent compound DuP 714. Substitution of D-MePhe of LY294468 by D-homoproline [Schacht, A. L.; Wiley, M. R.; Chirgadze, N.; Clawson, D.; Craft, T.].; Coffman, W. J, Jones N D, Gifford-Moore D, Olkowski J, Shuman R T, Smith G F and Weir L C. N-Substituted Glycines as replacements for Proline in Tripeptide Aldehyde Thrombin Inhibitors. Bioorg. Med. Chem. Lett 1995, 5, 2529-2534.] giving D-hPro-Pro-Arg-H was shown from crystallographic studies to retain the Gly-216 interaction, but with a 2 fold loss in affinity for thrombin (Kass_{Thr} 54×10⁵ and 20×10⁵ L/mol., respectively). Further substitution of the P2 proline by N-substituted glycine led to a further decrease in affinity for thrombin, but with some improved specificity; the most selective being D-hPro-(Nα-isopropyl)Gly-Arg-H (Kass_{Thr}/Kass_{Thy} 20, compared to 5.1 for LY294468). Replacement of P2 proline with N-substituted glycines in boronate based inhibitors retaining P3 D-Phe also reduced thrombin affinity, [Rupin, A.; Mennecier, P.; de Nanteuil, G. and Verbeuren, T. S18326 is a New Potent Anti-thrombin Agent which Does not Interfere with Fibrinolysis. Thromb. Haemostas. 1995; 73, 1309.] with a moderate improvement in specificity.

Non-Peptide Boronates

Non-peptide boronates have been proposed as inhibitors of proteolytic enzymes in detergent compositions. WO 92/19707 and WO 95/12655 report that arylboronates can be used as inhibitors of proteolytic enzymes in detergent compositions. WO 92/19707 discloses compounds substituted meta to the boronate group by a hydrogen bonding group, especially acetamido (—NHCOCH₃), sulfonamido (—NHSO₂CH₃) and alkylamino. WO 95/12655 teaches that ortho-substituted compounds are superior.

Non-peptide boronate inhibitors are to be preferred as serine protease inhibitors, insofar as they are in principle likely to be more readily synthesised in higher yield than boropeptides. In the pharmaceutical field, boropeptides can be disadvantageous also because of difficulties in providing adequate oral bioavailability.

Boronate enzyme inhibitors have wide application, from detergents to bacterial sporulation inhibitors to pharmaceuticals. In the pharmaceutical field, there is ample patent literature describing boronate inhibitors of serine proteases, for example thrombin, factor Xa, kallikrein, elastase, plasmin as well as other serine proteases like prolyl endopeptidase and Ig AI Protease. Thrombin is the last protease in the coagulation pathway and acts to hydrolyse four small peptides from each molecule of fibrinogen, thus deprotecting its polymerisation sites. Once formed, the linear fibrin polymers may be cross-linked by factor XIIIa, which is itself activated by thrombin. In addition, thrombin is a potent activator of platelets, upon which it acts at specific receptors. Thrombin also potentiates its own production by the activation of factors V and VIII.

Non-peptide boronic adds have many uses in areas outside enzyme inhibition, including synthesis, sensing, affinity chromatography and molecular recognition, immunoassays, carriers for transporting species through lipid bilayers.

The preparation and reactions of o-(cyanomethyl)benzeneboronic add have been described by Catlin and Snyder (Catlin, J. C. and Snyder, H. R. J. Org. Chem. 1969, 34, 1660-1663). Compounds which are described as derived from o-(cyanomethyl)benzeneboronic acid include o-(boronophenyl)acetamide [2-catechol-O—BPhCH₂CONH₂]. 2-(o-boronophenyl)ethylamine [2-B(OH)₂—PhCH₂CH₂NH₂] and certain N-substituted analogues of the latter, notably alpha-(piperidinocarbonyl)-o-tolueneboronic acid.

Arylboronic acids are very versatile and readily available derivatives and have manifold applications in areas as diverse as organometallic chemistry and organic synthesis (Beller 1998, Miyaura 1998, Murata 2000), host-guest chemistry and materials science (Tuladhar 1992; Hughes 1996; Kimura 1999), affinity chromatography (Singhal 1991; Biedryzycki 1992) and medicinal chemistry. (Keller, 1991; Hamachi, I.; Kimura, O.; Takeshita, H.; Shinkai, S. Chem. Lett. 1995. Westmark, 1996. Weston 1998, 41, 4577. (e) Brikh, 1999, and refs. cited therein):

Beller, M.; Fischer, Herrmann, W. A.; öfele, K.; Brossmer, C. Angew Chem. Int. Ed. Engl. 1995, 34, 1848.

Miyaura, N. Adv. Met. Org. Chem. 1998, 6, 187.

Murata, M.; Oyama, T.; Watanabe, S.; Masuda, Y. J. Org. Chem. 2000, 65, 164 and refs. cited therein.

Tuladhar, S. M.; D'Silva Tetrahedron Lett. 1992, 33, 265.

Hughes, M. P.; Shang, M.; Smith, B. D. J. Org. Chem. 1996, 61, 4510.

Kimura, T.; Yamashita, T.; Koumoto, K.; Shinkai, S. Tetrahedron Lett. 1999, 40, 6631. (b)

Singhal, R. P.; Ramamurthy, B.; Govindraj, N.; Sarwar, Y. J. Chromatogr. 1991, 543, 17. (b)

Biedryzycki, M.; Scouten, W. H.; Biedrzycka, Z. J. Organomet. Chem. 1992, 431, 255.

Keller, T. H.; Seufer-Wasserthal, P.; Bryan Jones, J. Biochem. Biophys. Res. Comm. 1991, 176, 401. (b) Hamachi, I.; Kimura, O.; Takeshita, H.; Shinkai, S. Chem. Lett. 1995, 529. (c)

Westmark, P. R.; Gardiner, S. J.; Smith, B. D. J. Am. Chem. Soc. 1996, 118, 11093. (d)

Weston, G. S.; Blázquez, J.; Baquero, F.; Shoichet, B, K. J. Med. Chem. 1998, 41, 4577. (e)

Brikh, A.; Morin, C. J. Organomet Chem. 1999, 581, 82 and refs. cited therein.

Other applications of non-peptide boronates, and potentially of their non-boronate analogues, are described in the following publications:

Boronate-functionalized polypyrrole as a new fluoride sensing material, Nicolas M, Fabre B, Simonet J, Chemical Communications, 1999, No.18, pp.1881-1882;

Boronate affinity adsorption of RNA: possible role of conformational changes, Singh N, Willson R C, Journal Of Chromatography A, 1999, Vol.840, No.2, pp.205-213;

Measurement of glycated haemoglobin by boronate-affinity high-pressure liquid chromatography, Stirk H, Allen K R, Annals Of Clinical Biochemistry, 1999, Vol.36, No.Pt2, pp.233-234;

Determination of glycated albumin by enzyme-linked boronate immunoassay (ELBIA), Ikeda K, Sakamoto Y, Kawasaki Y, Miyake T, Tanaka K, Urata T, Katayama Y, Ueda S, Horiuchi S, Clinical Chemistry, 1998, Vol.44, No.2, pp.256-263;

Novel separation material suitable for both boronate affinity chromatography and immobilized metal affinity chromatography, Liu X C, Mosbach K, Abstracts Of Papers Of The American Chemical Society, 1997, Vol.213, No.Pt1, pp.157 et seq;

Studies on oriented and reversible immobilization of glycoprotein using novel boronate affinity gel, Liu X C, Scouten W H, Journal Of Molecular Recognition, 1996, Vol.9, No.5-6, pp.462-467;

Selective monosaccharide transport through lipid bilayers using boronic acid carriers, Westmark P R, Gardiner S J, Smith B D, Journal Of The American Chemical Society, 1996, Vol.118, No.45, pp.11093-11100;

Study of a highly selective and sensitive analytical method based on the molecular recognition ability of boronic acid derivatives, Gamoh K, Bunseki Kagaku, 1996, Vol.45, No.1, pp.19-30;

Boronic adds facilitate the transport of ribonucleosides through lipid bilayers, Westmark P R, Smith B D, Journal Of Pharmaceutical Sciences, 1996, Vol.85, No.3, pp.266-269;

Coupling of m-aminophenylboronic acid to s-triazine-activated sephacryl—use in the affinity-chromatography of glycated hemoglobins, Bisse E, Wieland H, Journal Of Chromatography-Biomedical Applications, 1992, Vol.575, No.2, pp.223-228;

G T Morin, M P Hughes, M-F Paugam and B D Smith., transport of Glycosides through Liquid Organic membranes mediated by reversible boronate formation is a diffusion-controlled process. J. Am. Chem. Soc. 1994, 116, 8895-8901;

Boronic acids facilitate the transport of ribonucleosides through lipid bilayers

Weatmark, P R. and Smith, B D., J. Pharm. Sci., 1996, 85, 266-269.

Study of a highly selective and sensitive analytical method based on the molecular recognition ability of boronic acid derivatives, Gamoh, K. Bunseki Kagaku, 1996, 45, 19-30.

Isothiouronium-Substituted Benzene

Duzhak, V. G. Fiziol. Akt. Veshchestva. 1989, 21, 81-83 describes the structure and antiadrenergic properties of certain aralkylisothiouronium hydrogen halides, including the HCl salts of (4-N₂OPh)CH₂SC(NH)NH₂ and (2-OH, 4-N₂OPh)CH₂SC(NH)NH₂.

Despite the many advances in enzyme inhibitor and aryl boronate chemistry, their levels of commercialisation remain low and there remains a need to provide, in particular, a pool of, new non-peptide compounds useful as protease inhibitors and which can be drawn on for a wide range of applications. A particular benefit of non-peptide inhibitors is the potential ease and economy of their synthesis, whilst their small size in principle will enhance oral bioavailability. It would also be useful to provide non-peptide compounds with potential as affinity chromatography or molecular recognition agents, for example, and also as synthetic intermediates.

### BRIEF SUMMARY OF THE INVENTION

The present invention provides novel compounds, especially compounds useful as protease inhibitors, of the formula:

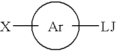

where:

Ar is a ring or ring system, which may be at least partially aromatic but can be non-aromatic and may be substituted by one or more moieties in addition to —X and -LJ;

X is a functional group which is a hydrogen-bond acceptor or a group transformed in vivo into a hydrogen bond acceptor, of which one example is BY¹Y² as defined below;

L is a linker selected from the group consisting of heteroatoms other than nitrogen (typically selected from 0 and preferably S) and chains of 1 to 15 carbon atoms (usually alkylenic carbon atoms) optionally interrupted and/or terminated by at least one heteroatom and especially by a single sulfur atom, which chain in some compounds has no side substituents (all carbon bonds outside the chain are to H) but in certain other compounds has side substituents, especially alkyl groups;

J is a moiety containing a basic nitrogen atom but not containing an amino acid residue.

The invention also provides novel synthetic methods which may be used in the preparation of the compounds of the invention as well as novel intermediates for making them.

### DETAILED DESCRIPTION OF THE INVENTION

The present invention provides novel compounds, especially compounds useful as protease inhibitors, and characterised in that they contain

1) a group Ar which is a ring or ring system, typically containing from 5 to 13 ring-forming atoms, and which may be non-aromatic (whether unsaturated or saturated, e.g. piperidinyl or cyclohexyl) but usually is an aryl or heteroaryl group, for example a fused ring system (e.g. naphthyl), a partially aromatic system (e.g. fluorenyl) or a 5- or 6-membered ring optionally containing one or, less preferably, more heteroatoms selected from N, O and S, of which pyridyl, quinolinyl and phenyl are preferred; and

2) substituents on Ar which comprise at least groups —X and -LJ.

For one class of serine protease inhibitors, Ar is most desirably phenyl or a wholly or partially hydrogenated analogue thereof; optionally as part of a fused ring system; such compounds desirably comprise X in a 1,4 or 1,3 relationship with the -L-J group.

X is a functional group which is a hydrogen-bond acceptor or a group transformed in vivo into a hydrogen bond acceptor (e.g. a protected hydrogen-bond acceptor), for example —NO₂, —CO₂E, —BY¹Y², CN or WHO, (—BY¹Y² is also potentially an electron acceptor),

where E is H, an ester-forming group (which typically contains from 1 to 10 carbon atoms and, for example, may be alkyl or alkenyl optionally interrupted by an ether, thioether or amino linkage and/or substituted by 1, 2, 3 or more moieties selected from halogen, hydroxy and amino), especially a pharmaceutically acceptable ester-forming group, or a cation, especially a pharmaceutically acceptable cation such as ammonium, sodium or potassium, for example; and

Y¹ and Y² are each independently —OR¹, —SR¹, halogen (especially —F) or —NR¹R² (of which hydroxy and alkoxy are most preferred), or Y¹ and Y² taken together with the boron atom form

a) a cyclic boron ester (i.e. Y¹ and Y² together form the residue of a compound having two hydroxy groups) which normally contains from 2 to 20 carbon atoms and from 0 to 3 heteroatoms selected from N, S and O,

b) a cyclic boron amide (i.e. Y¹ and Y² together form the residue of a compound having two amino groups) which normally contains from 2 to 20 carbon atoms and from 0 to 3 heteroatoms selected from N, S and O.

c) a cyclic boron amide-ester (i.e. Y¹ and Y² together form the residue of a compound having a hydroxy group and an amino group) which normally contains from 2 to 20 carbon atoms and from 0 to 3 heteroatoms selected from N, S and O,

and R¹ and R² are each independently an inert organic moiety, typically containing no more than 20 atoms which are not hydrogen or halogen. More preferably, R¹ and R² are each independently hydrogen or a moiety in which the non-hydrogen atoms are selected from the group consisting of C, N, O and S and number from 1 to 20 (especially 1, 2, 3, 4, 5, 6 or 7, for example methyl, ethyl, butyl, propyl) and which comprises at least one hydrocarbyl group which may be aliphatic or carbocyclic, and is for example selected from aryl, alkyl, alkylene, cycloalkyl, cycloalkylene, alkenyl, alkenylene, cycloalkenyl, cycloalkenylene, alkynyl and alkynylene (of which alkyl, alkylene, cycloalkyl and aryl form a preferred class), and optionally 1, 2 or 3 heteroatoms selected from O, N and S, and more preferably is hydrogen or C₁-C₁₀ alkyl and especially C₁, C₂, C₃ or C₄ alkyl, C₅-C₁₀cyclohydrocarbyl, (C₁-C₄)alkyl-(C₅-C₁₀)cyclohydrocarbyl or (C₅-C₁₀)cyclohydrocarbyl-(C₁-C₄)alkyl, or C₅-C₆ cyclohydrocarbyl (e.g. phenyl, cyclohexyl) substituted by up to three groups selected from C₁-C₄ alkyl, C₁-C₄ alkoxy and halogen, of which hydrogen is most preferred for one or both of R¹ and R². Those R¹ groups which contain alkylic carbon atoms may be interrupted at an alkylic carbon by an —O— linkage.

L is a linker selected from the group consisting of heteroatoms other than nitrogen (typically selected from O and preferably S) and chains of 1 to 15 carbon atoms (usually alkylic carbon atoms) optionally interrupted and/or terminated by at least one heteroatom and especially by a single sulfur atom, which chain in some compounds has no side substituents (all carbon bonds outside the chain are to H) but in certain other compounds has side substituents, especially alkyl groups. Preferably, L is of the formula —(CR⁵R⁶)ₗ-(Z)ₘ—(CHR⁷)ₙ—,

where each R⁵, R⁶ and R⁷ independently is —H, —OH, —NH₂, —SH, NCCH₂CO₂(C₁-C₁₄)alkyl or a moiety in which the atoms other than hydrogen and halogen are selected from the group consisting of C, N, O and S and number from 1 to 20 (especially 1, 2, 3, 4, 5, 6 or 7) and which comprises at least one hydrocarbyl group which is optionally substituted by halogen and may be aliphatic or carbocyclic, and is for example selected from aryl, alkyl, alkylene, cycloalkyl, cycloalkylene, alkenyl, alkenylene, cycloalkenyl, cycloalkenylene, alkynyl and alkynylene (which may be substituted by halogen and of which alkyl, alkylene, cycloalkyl and aryl form a preferred class), and 0, 1, 2 or 3 heteroatoms selected from O, N and S; l is from 0 to 6 and preferably 1, 2, 3 or 4, usually 2 or, most usually, 1; Z is O, N or preferably S; and m is 0 or, preferably, 1; n is from 0 to 6 and usually 0 but sometimes 1 or 2, for example; provided that (l+m+n) is at least 1. Preferably, each R⁵, R⁶ and R⁷ independently is hydrogen or an optionally substituted moiety selected from the group consisting of C₁-C₈ alkyl, C₁-C₈ alkenyl, C₁-C₈ alkynyl (which C₁-C₈ moieties for example contain 1, 2, 3, 4, 5 or 6 carbon atoms), C₅-C₁₀ cyclohydrocarbyl (especially phenyl or cyclohexyl), (C₁-C₄)alkyl-(C₅-C₁₀)cyclohydrocarbyl, and (C₅-C₁₀)cyclohydrocarbyl-(C₁-C₄)alkyl, where cyclohydrocarbyl is preferably phenyl or cyclohexyl, and said moieties when terminated by an ether, thioether or amino (—NH—) linkage to the remainder of L and/or, in the case of moieties containing at least one allylic carbon atom, interrupted at an alkylic carbon atom by a said linkage, the optional substitution being by halogen or by an OH, SH or NH₂ group.

Normally, there is no more than one R⁵ group which is not hydrogen and in one class of compounds the or each R⁵ group is hydrogen. Similarly, there is typically no more than one R⁶ group which is not hydrogen and often the or each R⁶ group is hydrogen. In one class of compounds, the total of R⁵ plus R⁶ groups which is not hydrogen is 2, in a second class of compounds it is 1 and in another class of compounds it is 0. Usually, there is no more than one R⁷ group which is not hydrogen and often the or each R⁷ group is hydrogen. The total of (R⁵ plus R⁶ plus R⁷) groups which is not hydrogen is 3 in a preferred class of compounds but is 2 in a more preferred class of compounds and 1 in another preferred class of compounds, whilst a particularly preferred class of compounds has all its R⁵, R⁶ and R⁷ groups as hydrogen. Of the non-hydrogen R¹ and R⁶ options, C₁-C₈ alkyl (e.g. CL, C₂, C₃, C₄, C₅ or C₆ alkyl) and phenyl or cyclohexyl are most preferred and also alkoxy (e.g. methoxy), alkylthio (e.g. methylthio), phenoxy and phenylthio. Of the non-hydrogen R⁷ options, C₁-C₈ alkyl is most preferred (e.g. C₁, C₂, C₃, C₄, C₅ or C₆ alkyl), especially methyl. Preferred —(CR⁵R⁶)ₗ— moieties are —CH₂— and —CHalkyl-, preferred —(CHR⁷)ₙ— moieties include —CH₂— and —CHalkyl-; in both cases alkyl is C₁-C₈ and a preferred alkyl group is methyl.

J is a moiety containing a basic nitrogen atom but not containing an amino add residue, and preferably is of the formula

(i) -GNR³R⁴ (e.g. —C(O)NR³R⁴—C(O)NHR¹),

(ii) -GNR¹OH,

(iii) -GNR¹C(NR¹)H, especially -GNHC(NH)H,

(iv) -GNR¹C(NR¹)NR¹OH, especially -GNHC(NH)NHOH,

(v) -GNR¹C(NR¹)NR¹CN, especially -GNHC(NH)NHCN,

(vi) -GNR¹C(NR¹)NR¹COR¹, especially -GNHC(NH)NHCOR¹,

(vii) -GNR¹C(NR¹)NR¹R², especially -GNHC(NH)NHR¹

(viii) -GC(NR¹)NR¹R², especially -GC(NH)NHR¹,

(ix) -GC(NR¹)NR¹NR¹COR¹, especially -GC(NH)NHNHCOR¹,

(x) -GC(NR¹)NR¹C(NR¹)NR¹R², especially -GC(NH)NHC(NH)NH₂,

(xi) -GC(NR¹)NR¹COR¹, especially -GC(NH)NHCOR¹,

(xii) -GNR¹C(O)R¹, especially -GNHC(O)R¹ (e.g. —CONHC(O)R¹),

(xiii) —OC(O)NR¹R², especially —OC(O)NHR¹,

(xiv) —OC(O)NR¹C(O)R¹, especially —OC(O)NHC(O)R¹,

(xv) —C(O)ONR³R⁴,

(xvi) -GN(R¹)COOR¹, especially —CON(R¹)COOR

(xvii) -GN(COOR¹)C(NH₂)═NCOOR¹, especially —N(COOR)C(NH₂)═NCOOR¹,

where:

G is nothing or is SO₂, CO or CO(CH₂)ₚCO, where p is 1, 2, 3 or 4 (and preferably 2), of which it is preferred for G to be nothing or, less commonly, CO,

R¹ and R² are as defined above and each R¹ group of a compound having a plurality of R¹ groups is selected from the R¹ options independently of the other R¹ group(s) of the compound, R¹ and R² usually being selected from H, alkyl (e.g. methyl or ethyl) and, less frequently, phenyl or cyclohexyl and one or both of R¹ and R² typically being H in compounds containing the moiety NR¹R², and

R³ and R⁴ are each independently hydrogen or a moiety in which the atoms other than hydrogen and halogen are selected from the group consisting of C, N, O and S and number from 1 to 20 (especially 1, 2, 3, 4, 5, 6 or 7) and which comprises at least one hydrocarbyl group which is optionally substituted by halogen and may be aliphatic or carbocyclic, and is for example selected from aryl, alkyl, alkylene, cycloalkyl, cycloalkylene, alkenyl, alkenylene, cycloalkenyl, cycloalkenylene, alkynyl and alkynylene (which may be substituted by halogen and of which alkyl, alkylene, cycloalkyl and aryl form a preferred class), and optionally 1, 2 or 3 heteroatoms selected from O, N and S, and more preferably said moiety is:

C₁-C₁₀ alkyl and especially C₁, C₂, C₃, C₄, C₅ or C₆ alkyl;

a substituted C₁-C₁₀ (e.g. C₁, C₂, C₃, C₄, C₅ or C₆) alkyl moiety and especially alkyl substituted by carboxyl, alkoxycarbonyl (wherein the alkoxycarbonylalkyl group contains from 1 to 12 carbon atoms, e.g. 1, 2, 3, 4, 5 or 6), alkoxy (wherein the alkoxylalkyl group contains from 1 to 12 carbon atoms, e.g. 1, 2, 3, 4, 5 or 6), hydroxy or halogen;

C₅-C₁₀ cyclohydrocarbyl (especially phenyl or cyclohexyl) optionally substituted by a group selected from C₁, C₂, C₃ or C₄ alkyl, C₁, C₂, C₃ or C₄ alkoxy and halogen;

(C₅-C₁₀)cyclohydrocarbyl-(C₁-C₄)alkyl whose cyclohydrocarbyl part is preferably phenyl or cyclohexyl and is optionally substituted by a group selected from C₁, C₂, C₃ or C₄ alkyl, C₁, C₂, C₃ or C₄ alkoxy and halogen; or

C₅-C₆ cyclohydrocarbyl (e.g. phenyl or cyclohexyl) substituted by two or three groups selected from C₁, C₂, C₃ or C₄ alkyl, C₁, C₂, C₃ or C₄ alkoxy and halogen.

Alternatively, R³ and R⁴ together with their attached N form a ring, especially a 5- or 6-membered ring (such as imidazolyl, oxazolyl or thiazolyl, for example) which is optionally part of a fused ring system (e.g. a C₉-C₁₀ fused ring such as, for example, benzoxazolinyl or thiazolinyl) and/or substituted. Preferred substituents are: C₁-C₁₀ alkyl and especially C₁, C₂, C₃, C₄, C₅ or C₆ alkyl; a substituted C₁-C₁₀ (e.g. C₁, C₂, C₃, C₄, C₅ or C₆) alkyl moiety and preferably alkyl substituted by carboxyl, alkoxycarbonyl (wherein the alkoxycarbonylalkyl group contains from 1 to 12 carbon atoms, e.g. 1, 2, 3, 4, 5 or 6), alkoxy (wherein the alkoxylalkyl group contains from 1 to 12 carbon atoms, e.g. 1, 2, 3, 4, 5 or 6), hydroxy or halogen; (C₁-C₄)alkyl-(C₅-C₁₀)cyclohydrocarbyl; C₅-C₁₀ cyclohydrocarbyl (where cyclohydrocarbyl is an aromatic or non-aromatic ring or ring system, especially a 5- or 6-membered ring, for example an aromatic heterocycle, phenyl or cyclohexyl), which is optionally substituted by, in particular, 1, 2 or 3 moieties selected from the group consisting of 5- and 6-membered rings (e.g. phenyl, cyclohexyl or an aromatic heterocycle) hydroxy, amino, C₁, C₂, C₃ or C₄ alkyl, C₁, C₂, C₃ or C₄ alkoxy, thiol, C₁, C₂, C₃ or C₄ alkylthio, amino, nitrile, carboxy, —CHO, —C(O)—(C₁-C₄)alkyl, —SO₂, substituted amino (e.g. mono- or di-alkylamino or alkylamido, where alkyl typically has from 1 to 4 carbon atoms) and halogen and is preferably an aromatic ring such as phenyl, pyridinyl or pyrimidinyl. The aforesaid alkyl-, cyclohydrocarbyl- and ring-containing substituents may be terminated by an ether or thioether linkage to the moiety which they substitute and/or, in the case of substituents containing at least one alkylic carbon atom, interrupted at an alkylic carbon by an ether or thioether linkage, especially to form an alkoxy substituent. All the organic moieties mentioned in this paragraph may be substituted by halogen; they may all be substituted by hydroxy. thiol or amino.

One preferred class of rings formed by R³ and R⁴ comprises 6-membered rings substituted at their 4-position by alkyl, alkoxy or by an optionally substituted 5- or 6-membered ring (which is usually an aromatic ring); the optional substituent is typically at the 2-position and is exemplified by alkyl or alkoxy. Another preferred class of rings formed by R³ and R⁴ comprises 6-membered saturated nitrogen heterocycles (for example piperidine or piperazine) substituted at their 4-position by an optionally substituted 6-membered (hetero)aromatic ring, especially phenyl, pyridinyl or pyrimidinyl; the optional substituent is typically at the 2-position and is exemplified by alkyl or alkoxy. All the rings mentioned in this paragraph may be C-substituted by halogen.

One or more of R¹, R², R³ and R⁴, when attached to nitrogen, may in any event be an N-protecting group.

Alternatively, -LJ is of the formula —C═N—NR¹—C(NR¹)NR¹R², especially —C═N—NH—C(NH)NH₂.

In one preferred class of compounds, -LJ comprises a heterocycle, notably a nitrogen-containing heterocycle, which is protonated at physiological pH, i.e. has a pKa of from about 7 to about 9. pKa for a molecular functional group is defined as the value of pH at which 50% of the groups in solution are charged and 50% are neutral. pKa can be calculated from the negative logarithm of the value of the equilibrium constant Kₐ for the equation for reaction of an acid HA with a base B to give the conjugate base HB and the conjugate add A, i.e.

HA+B═BH⁺+A⁻

Where

Kₐ=[HB⁺][A⁻]/[HA][B]

Ref. M. B. Smith, ‘Organic Synthesis’, Chapter 2, page 97, McGraw Hill international series, 1994. ISBN 0-07-113909-5.

Suitable heterocycles having a pKa of 7-9 include imidazole and analogous structures, for example benzoxazole and benzimidazole. As examples, there may be mentioned:

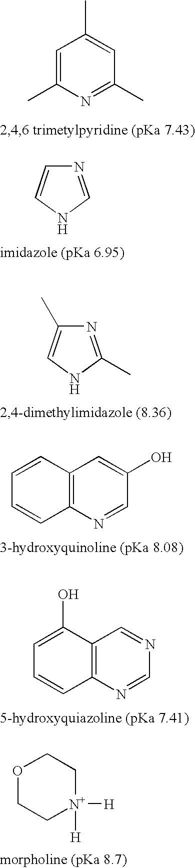

Ref: CRC Handbook of chemistry and Physics, R. C. Weast (ed), 68^{th} edition, 1987, pD-159, CRC Press, Inc., Boca Raton, Fla.

Of the compounds falling within the above definition, the following aralkylisothiouronium compounds are known and described as having antiadrenergic properties (Duzhak, V. G. Fiziol. Akt Veshchestva. 1989, 21, 81-83):

(4-N₂OPh)CH₂SC(NH)NH₂

(2-OH, 4-N₂OPh)CH₂SC(NH)NH₂.

These known compounds are excluded from the scope of the compounds of the invention. However, the invention does include these compounds in other aspects, notably the use of these known compounds as serine protease inhibitors and methods of treatment which use the known compounds to treat disorders suceptible to treatment by inhibition of a serine protease.

Also known are compounds of the following formulae (Catlin, J. C. and Snyder, H. R. J. Org. Chem. 1969, 34, 1660-1663)

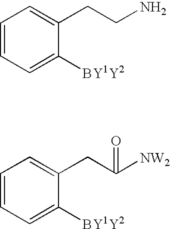

where

each W is H, or

one W is H and the other is the residue of 2-aminophenol, or

both W groups together with the adjoining N form piperidine, and

Y¹ and Y² are both H or together form the residue of catechol.

These known benzene boronic acid derivativess are excluded from the scope of the compounds of the invention. However, the invention does include these compounds in other aspects, notably pharmaceutical compositions and other protease inhibitory or pharmaceutical aspects, for example the use of the compounds as serine protease inhibitors and methods of treatment which use the known compounds to treat disorders suceptible to treatment by inhibition of a serine protease.

The compounds of the invention can exist in different forms, such as acids, esters, salts and tautomers, for example, and the invention includes all variant forms of the compounds. In particular, the compounds may be in the form of add addition salts which, for those compounds for pharmaceutical use, will be pharmaceutically acceptable. Exemplary acids include HBr, HCl and HSO₂CH₃.

Those compounds which are boronates may in particular have their boronate groups in the form of adds, salts or esters. Amines of the diamine type may have disadvantages as cations for boronate salts for pharmaceutical compositions, due to their potential toxicity. Diamines will tend to form cyclic derivatives. As a favourable point, while promoting transport into the membrane, cyclic boronates may be labile enough to dissociate and promote elution into the vascular lumen of the free add

The invention includes prodrugs for the active pharmaceutical species of the invention, for example in which one or more functional groups are protected or derivatised but can be converted in vivo to the functional group, as in the case of derivatised boronate groups convertible in vivo to —B(OH)₂ (which representation includes of course tetrahedral boronate species), or in the case of protected nitrogens. The term “prodrug,” as used herein, represents compounds which are rapidly transformed in vivo to the parent compound, for example, by hydrolysis in blood. A thorough discussion is provided in T. Higuchi and V. Stella, Pro-drugs as Novel Delivery Systems, Vol. 14 of the A.C.S. Symposium Series, Edward B. Roche, ed., Bioreversible Carriers in Drug Design, American Pharmaceutical Association and Pergamon Press, 1987, and Judkins, et al. Synthetic Communications, 26(23), 43514367 (1996), each of which is incorporated herein by reference.

The use of protecting groups is fully described in ‘Protective Groups in Organic Chemistry’, edited by J W F McOmie, Plenum Press (1973), and ‘Protective Groups in Organic Synthesis’, 2nd edition, T W Greene & P G M Wutz, Wiley-Interscience (1991).

Thus, it will be appreciated by those skilled in the art that, although protected derivatives of compounds of the invention (e.g compounds of formula (I), (II), (III), (IV) or (V)) may not possess pharmacological activity as such, they may be administered, for example parenterally or orally, and thereafter metabolised in the body to form compounds of the invention which are pharmacologically active. Such derivatives are therefore examples of “prodrugs”. All prodrugs of the described compounds are included within the scope of the invention.

The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or add in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., US, 1985, p. 1418, the disclosure of which is hereby incorporated by reference.

The invention thus includes pharmaceutically-acceptable salts of the disclosed compounds wherein the parent compound is modified by making acid or base salts thereof. for example the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

The phrase “pharmaceutically acceptable” is employed herein to refer to those compounds, materials, compositions, and/or dosage forms which are, within the scope of sound medical judgment, suitable for use in contact with the tissues of human beings or animals without excessive toxicity, irritation; allergic response, or other problem or complication, commensurate with a reasonable benefit/risk ratio.

Similarly, many of the groups referred to or featured herein (especially those containing heteroatoms and conjugated bonds) can exist in tautomeric forms and all these tautomers are included in the scope of the invention. There is a debate in the literature as to whether boronates in aqueous solution form the ‘trigonal’ B(OH)₂ or ‘tetrahedral’ B(OH)⁻₃ boron species, but NMR evidence seems to indicate that at a pH below the pKa of the boronic add the main boron species is the neutral B(OH)₂. In the stomach/upper duodenum the pH is likely to be between 6 and 7, so the trigonal species is likely to be predominant here. In any event, the symbol —BY¹Y² includes tetrahedral as well as trigonal boron species.

The compounds of formula I may also contain one or more asymmetric carbon atoms and may therefore exhibit optical and/or diastereoisomerism. All diastereoisomers may be separated using conventional techniques, e.g. chromatography or fractional crystallisation. The various stereoisomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation or epimerisation, or by derivatisation, for example with a homochiral acid followed by separation of the diastereomeric derivatives by conventional means (e.g. HPLC, chromatography over silica). All stereoisomers are included within the scope of the invention.

Geometric isomers may also exist in the compounds of the present invention. The present invention contemplates the various geometric isomers and mixtures thereof resulting from the arrangement of substituents around a carbon-carbon double bond and designates such isomers as of the Z or E configuration, wherein the term “Z” represents substituents on the same side of the carbon—carbon double bond and the term “E” represents substituents on opposite sides of the carbon—carbon double bond.

The invention therefore includes all variant forms of the defined compounds, for example any tautomer or any pharmaceutically acceptable salt, ester, acid or other variant of the defined compounds and their tautomers as well as substances which, upon administration, are capable of providing directly or indirectly a compound as defined above or providing a species which is capable of existing in equilibrium with such a compound.

The term “alkyl” in this specification includes linear and branched alkyl groups, for example methyl, ethyl, n-propyl, iso-propyl, tert-butyl, n-pentyl and n-hexyl. Similarly, the term “alkoxy” includes groups of which the alkyl part may be linear or branched, for example one of those groups listed in the preceding sentence; alkylene groups may likewise be linear or branched and may, for example, correspond to one of those alkyl groups listed in the preceding sentence. The alkyl groups may be substituted by inert substituents, notably halogen.

The term cyclohydrocarbyl includes saturated or unsaturated cyclic hydrocarbyl groups (particularly aryl, notably phenyl, or cycloalkyl, notably cyclohexyl).

The term “halogen” herein includes reference to F, Cl, Br and I, of which Cl is often preferred. In one class of compounds, Br is preferred.

It will be understood that the invention specifically includes variants of preferred or exemplary compounds in which one or more moieties have been replaced by alternatives described in this application. By way of example, the substituents and/or aryl group of formula VII below may be replaced by alternatives described herein for moieties at such positions.

Throughout the description and claims of this specification, the words “comprise” and “contain” and variations of the words, for example “comprising” and “comprises”, means “including but not limited to”, and is not intended to (and does not) exclude other moieties, additives, components, integers or steps.

The identity of any further substituent groups on group Ar is not critical to the invention, though in general there will often be 1, 2, 3 or 4 further substituents, typically selected from the group consisting of: -LJ moieties (one class of compounds has two -LJ moieties which may be different but are preferably the same), X moieties (one class of compounds has two X moieties which may be the same but are often different), 3 moieties, -linker-RING or -linker-RING-linker-RING (as defined subsequently), -linker-D (as defined subsequently), halogen, hydroxy, a hydroxy derivative (e.g. alkoxy), thiol, alkylthio, amino, nitrile, carboxy, —CHO, C(O)alkyl, —SO₂, and substituted amino (e.g. mono- or di-alkylamino or alkylamido), and moieties comprising at least one group (e.g. 1, 2, 3 or 4 groups, 1, 2 or 3 groups, 1 or 2 groups or one group) selected from substituted or unsubstituted aliphatic, alicyclic and (hetero)aromatic groups; said moieties typically contain 1 to 30 and often 1 to 20 carbon atoms and usually the atoms other than hydrogen and halogen (e.g. selected from the group consisting of C, N, O and S) number from 1 to 20 (especially 1, 2, 3, 4, 5, 6 or 7). Said moieties may, for example, be terminated or interrupted by heteroatom-containing linkages (of which ether and carbonyl linkages may be mentioned as examples). Some substituent groups on Ar are substituted or unsubstituted moieties containing at least two groups selected from optionally substituted aliphatic, alicyclic and (hetero)aromatic groups; exemplary substituent groups on Ar are alkyl, aralkyl or arylalkyl, all of which may be substituted or may be linked to Ar by, and/or interrupted by, an ether or thioether linkage. As substituents for the aliphatic, alicyclic and (hetero)aromatic moieties there may be mentioned halogen (often F, Cl or Br), hydroxy, a hydroxy derivative (e.g. alkoxy), thiol, alkylthio, amino, nitrile, carboxy, —CHO, —C(O)alkyl, —SO₂, and substituted amino (e.g. mono- or di-alkylamino or alkylamido), of which halogen is preferred. In one class of compounds, two of the substituent groups together form a cyclic group, which is usually a 5- or 6-membered structure optionally containing at least one heteroatom and in some classes of compounds is fused to a further ring, especially to a substituted or unsubstituted benzene ring (suuitable substituents include those in the preceding list of substituents). The alkyl groups mentioned in this paragraph, or the alkyl part of alkyl-containing moieties, may contain, for example from 1 to 10 carbon atoms, especially from 1 to 6 carbon atoms and most preferably 1, 2, 3 or 4 carbon atoms.

In some preferred compounds, Ar is further substituted by a single additional substituent which is a Q group as defined below and is typically at the 2- or 4 position to X.

A first class of compounds has an -LJ group in the 2-position relative to an —X group.

A second class of compounds has an -LJ group in the 3-position relative to an —X group.

A third class of compounds has an -LJ group in the 4-position relative to an —X group.

As has been stated, X is a hydrogen bond acceptor or a group which can be transformed into one in vivo; the invention therefore includes prodrugs in which X is a group converted in the body to a hydrogen bond acceptor. As a specific example may be mentioned boronate esters which have been found to have in vitro activity dose to that of the corresponding acid, which is believed to result from hydrolysis of the ester to its corresponding acid, which is the active agent.

Particularly preferred, therefore, are compounds in which X is —BY¹Y², where Y¹ and Y² are preferably H or a group which may be hydrolysed (e.g. in vivo) to form —OH, especially alkoxy or the residue of a diol. A preferred diol is pinacol and other exemplary diols include pinanediol, neopentylglycol, diethanolamine, 1,2-ethanediol, 1,2-propanediol, 1,3-propanedlol, 2,3-butanediol, 1,2-diisopropylethanediol, 5,6-decanediol and 1,2-dicyclohexylethanediol.

Another preferred X group is —NO₂.

One preferred class of compounds useful as serine protease inhibitors comprises those having an -L- group of the formula —(CR⁵R⁶)ₗ-(Z)ₘ—(CHR⁷)ₙ in which m is 1 and, of these, a preferred subclass of compounds have Z as S; a particularly preferred L group is therefore —CH²—S— and another preferred L group is —CHMe—S. Typically, in compounds of this class and sub-class (like other compounds of the invention) (l+m+n) is from 2 to 8 and preferably 2, 3, 4 or 5 (e.g. 2 or 3); l is most preferably 1 or 2 and n is often 0 (and if not 0, usually 1).

Compounds in which -LJ is CH₂NH₂ are poor inhibitors of thrombin and a preferred class of compounds excludes those in which -LJ is —CH₂NH₂ or an analogous group of the formula —CH₂NR¹R². More generally, it is preferred that L is not CH₂, especially when J does not contain a G moiety which is SO₂, CO or CO(CH₂)ₚCO.

Sometimes it is preferred that -LJ is not aminoalkyl or amidoalkyl.

As exemplary J groups in compounds useful as serine protease inhibitors there may be mentioned -GNH₂, -GNHalkyl, -GNHCH₂carboxyalkyl, -GN(alkyl)₂ (where G is as defined above and is usually CO or, more usually, nothing) and moieties of the following structure:

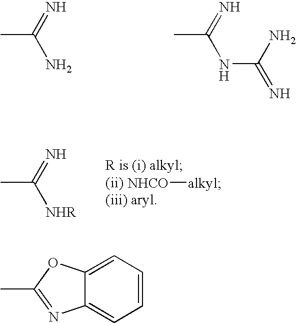

In these exemplary J groups, “alkyl” and “alkoxy” preferably have 1, 2, 3, 4, 5 or 6 carbon atoms and especially have one carbon or two carbons, and “aryl” is preferably phenyl or phenyl substituted by C₁, C₂, C₃ or C₄ alkyl or C₁, C₂, C₃ or C₄ alkoxy. All these preferred 3 groups may be bonded to any L group, especially one of the preferred L groups described herein, for example —CH₂—S— or, in the case of those 3 groups linked to L from a carbonyl moiety of J, —(CH₂)—, particularly —CH₂—CH₂—. A most preferred J group for all serine protease inhibitors is —C(NH)NHR″, where R″ is H, C₁-C₈ alkyl, cycloalkyl (e.g. C₅-C₆) or phenyl and preferably is H or alkyl, where alkyl is preferablu C₁, C₂, C₃ or C₄ alkyl, for example ethyl or methyl.

As will now be apparent to the reader, for all serine protease inhibitors, a particularly preferred -L-J group is —(CR⁵R⁶)ₗ—S—(CHR⁷)ₙ—C(NH)NHR″ more preferably —(CR⁵R⁶)ₗ—S—C(NH)NHR″, and most preferably —CHR⁶—SC(NH)NHR″. Especially preferred are such compounds in which X is BY¹Y². More desirably still, Ar is phenyl or a wholly or partially hydrogenated analogue thereof; optionally as part of a fused ring system; such compounds desirably comprise BY¹Y² or other X group in a 1,4 or 1,3 relationship with the -L-J group. Preferred values for the variables referred to in this paragraph have previously been described.

As has already been described, Ar is a ring or ring system and especially an aryl or heteroaryl group, and often contains up to 13 ring-forming atoms. The terms “aryl” and “heteroaryl” include reference to ring sytems which are partly aromatic, for example fluorenyl, and suitable aryl groups include phenyl, fluorenyl, biphenyl and naphthyl. Useful heteroaryl groups include 5-, 6-, 9- and 10-membered mono- or bicyclic aromatic rings which optionally contain 1, 2 or 3 heteroatoms selected from N, O and S. As exemplary heteroaryl moieties there may be mentioned pyridyl (2-, 3- or 4-pyridyl); furyl (2- or 3-furyl); 2- or 3-benzofuranyl; 2- or 3-thiophenyl; 2- or 3-benzo[b]thiophenyl; 2- or 3- or 4-quinolinyl; 1-, 3- or 4-isoquinolinyl; 2- or 3-pyrrolyl; 1-, 2- or 3-indolyl; 2-, 4- or 5-oxazolyl; 2-benzoxazolyl; 2-, 4- or 5-imidazolyl; 1- or 2-benzimidazolyl; 2-, 4- or 5-thiazolyl; 2-benzothiazolyl; 3-, 4- or 5-isoxazolyl; 3-, 4- or 5-pyrazolyl; 3-, 4- or 5-isothiazolyl; 3- or 4-pyridazinyl; 2-,4- or 5-pyrimidinyl; 2-pyrazinyl; 2-triazinyl; 3- or 4-cinnolinyl; 1-phthalazinyl; 2- or 4-quinazolinyl or 2-quinoxalinyl. More desirably still, Ar is phenyl or a wholly or partially hydrogenated analogue thereof; optionally as part of a fused ring system.

Ar may be aryl or a wholly or partially hydrogenated aryl analogue, e.g. a cycloalkane. More preferably Ar is phenyl a wholly or partially hydrogenated analogue thereof, i.e. particularly preferred compounds, in particular useful as serine protease inhibitors, are of formula I:

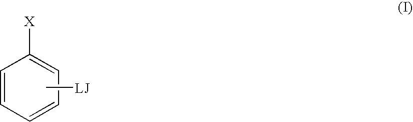

In which 1, 2, 3 or 4 of the remaining benzene ring carbon atoms is/are optionally substituted as described above. In one class of compounds, there is a single additional substituent at the 2, 3, or 4 position relative to X. As already discussed, the invention includes (i) compounds in which -LJ is meta to X, (ii) compounds in which is para to X, and (iii) compounds in which -LJ is ortho to X.

The benzene ring in formula (I) above as well as following formulae (U) to (VII), (XIIIA), (XIIIB) and (XIV) may be replaced by a wholly or partially hydrogenated analogue, in particular, cyclohexane.

Thus, one class of compounds, in particular useful as serine protease inhibitors, is of formula II:

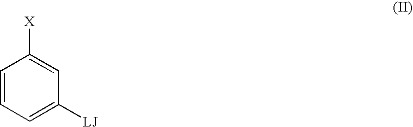

where 0, 1, 2, 3 or 4 of the remaining ring carbons are substituted as described above.

Preferred -LJ groups are of course as discussed above (—(CR⁵R⁶)ₗ-(Z)ₘ—(CHR⁷)ₙ— wherein m is 1 and Z is S in many). Preferred sub-classes of formula II compounds are of formulae III, IV and V (and those corresponding to formula IV where Q is 4- to X):

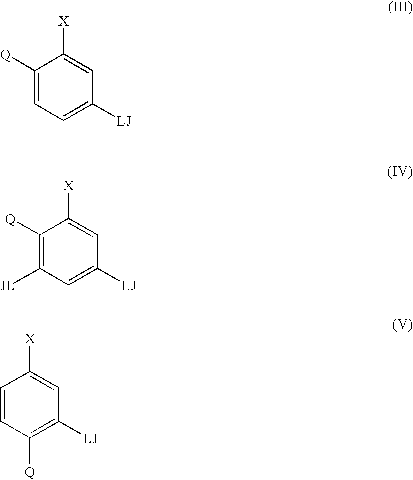

where

the ring carbons are bonded to H or, less preferably, halogen unless otherwise shown,

the two -LJ groups of formula (IV) are the same or different, and

Q is selected from the group consisting of: H; X moieties (in which case the two X moieties of the compound may be the same but are often different); J moieties; -linker-RING (as defined in the following paragraph); -linker-RING-linker-RING; -linker-D (as defined in the following paragraph); halogen; hydroxy; hydroxy derivatives (e.g. alkoxy); thiol; alkylthio; substituted or unsubstituted aliphatic, alicyclic or (hetero)aromatic groups; and substituted or unsubstituted moieties containing at least two groups (e.g. 2, 3 or 4, such as 2 or 3) selected from aliphatic, alicyclic and (hetero)aromatic groups and optionally one or more (e.g. 1, 2 or 3) linking groups interconnecting adjacent ones of said at least two groups, suitable linking groups including M moieties as defined below and especially —O— or —CO—. As substituents for the aliphatic, alicyclic and (hetero)aromatic moieties there may be mentioned halogen, hydroxy, a hydroxy derivative (e.g. alkoxy), thiol, alkylthio, amino, nitrile, carboxy, —CHO, —C(O)alkyl, —SO², and substituted amino (e.g. mono- or di-alkylamino or alkylamido), of which halogen is preferred. Q typically contains no more than 30, preferably no more than 20, carbon atoms and often no more than 16 carbon atoms; for example, Q groups may contain no more than 20, preferably no more than 16 atoms atoms which are other than hydrogen or halogen. Aliphatic groups are typically alkyl or alkenyl and may contain, e.g. 1-20, preferably 1-16 and optionally 1-6 (e.g. 1, 2, 3 or 4) carbon atoms whilst cyclic structures are typically 5- or 6-membered monocyclic or 10-12 membered-bicyclic structures, where any heteroatoms are usually O, N or S. The alkyl groups mentioned in this paragraph, or the alkyl part of alkyl-containing moieties, may contain, for example from 1 to 10 carbon atoms, especially from 1 to 6 carbon atoms and most preferably 1, 2, 3 or 4 carbon atoms.

As some particularly preferred Q groups there may be mentioned hydrogen or moieties of the structure:

-linker-RING, -linker-RING-linker-RING or -linker-D

where

linker is a bond, —O—, —NH—, —NH-M-, -M- or C₁, C₂, C₃ or C₄ alkylene optionally interrupted and/or terminated by one or more —C— or —NH— linkages, where M is SO₂, CO, (CH²)_{q}SO₂, (CH₂)_{q}CO, CO(CH₂)_{q}CO [especially CO(CH₂)₂CO] or CHMeCO, where q is 1, 2, 3, or 4. Preferred linkers are a bond, —O—, —NH—, —NH-M-, -M- where M is preferably CO.

Each RING independently is a mono- or bi-cyclic ring which may be wholly or partially aromatic (e.g. phenyl or cyclohexyl, piperazinyl, naphthyl, pyrridyl) additionally substituted by 0, 1, 2 or 3 substituents selected from hydrocarbyl (especially alkyl or alkenyl), halogen, hydroxy, a hydroxy derivative (e.g. alkoxy), thiol, alkylthio, amino, nitrile, carboxy, —CHO, —C(O)alkyl, —SO₂R¹ (where R¹ is as defined above and especially alkyl), —SO₂aa (where aa is a natural or unnatural amino acid), and substituted amino (e.g. mono- or di-alkylamino or alkylamido), of which halogen is most preferred. The hydrocarbyl and alkyl groups mentioned in this paragraph, or the alkyl part of alkyl-containing moieties, may be substituted by halogen; they may be interrupted by one or a plurality of ether linkages and typically contain, for example, from 1 to 10 carbon atoms, especially from t to 6 carbon atoms and most preferably 1, 2, 3 or 4 carbon atoms, such as ethyl or methyl.

Where Q is -linker-RING-linker-RING, each RING is preferably a monocyclic ring and usually a 5-membered or preferably 6-membered ring, for example a non-aromatic heterocycle or, in other embodiments, phenyl. Where Q is -linker-RING-linker-RING, each linker independently is preferably a bond, —O—, —NH—, —NH-M-, M where M is preferably CO and most preferably is a bond or, in another class of compounds, —O—. Some preferred -linker-RING-linker-RING moieties comprise linker attached to a first monocyclic (especially 6-membered) ring (often a non-aromatic heterocycle) attached to a second monocyclic (especially 6-membered) ring, the second ring preferably being aromatic, for example benzene or pyridine, and sometimes being substituted by a single further substituent

In some compounds Q is -linker-RING-linker-RING and each RING is a 6-membered monocyclic ring, the monocyclic rings being the same or different; in certain compounds both are phenyl. In particular preferred compounds of this type, the RING of the -linker-RING-linker-moiety is 1,4-substituted by the two linker moieties; additionally or alternatively each ring independently may be further substituted as previously described, usually by no more than a single further substituent per ring which is typically halogen, alkyl, hydroxy, alkoxy, thiol, alkylthio, amino, alkylamino, nitrile, carboxy, —CHO, —C(O)alkyl, the alkyl groups (including the alkyl part of alkyl-containing moieties) containing from 1 to 10 carbon atoms, e.g. 1, 2, 3 or 4, for example methyl or ethyl, and optionally being interrupted by one or a plurality of ether linkages.

In other compounds, Q is linker-RING where RING is a monocyclic ring substituted at the 4-position or 2-position to linker by a non-cyclic substituent as previously described and typically as described in the previous paragraph.

D is a C₁-C₁₆, preferably C₁-C₆, moiety constituted by alkyl, alkylene, cycloalkyl and/or cycloalkylene residues wherein the alkyl and alkylene residues may be interrupted by, or linked to an adjacent residue by, an —O— linkage and/or be substituted by —CHO, —SO₂R, or a residue of a natural or unnatural amino acid, for example D may be methyl or cyclohexyl.

One class of Q groups which is preferred is uncharged at physiological pH. This class includes Q groups containing heterocyclic structures which have a pKa of less than 7, for example pyrazine (0.65), pyridine (5.3) and quinoline (4.0).

One class of Q groups is of the formula -linker-RING, where linker is —O—; another class of Q groups is of the formula -linker-RING, where linker is —NHCO—:

Some preferred classes of Q groups are shown below:

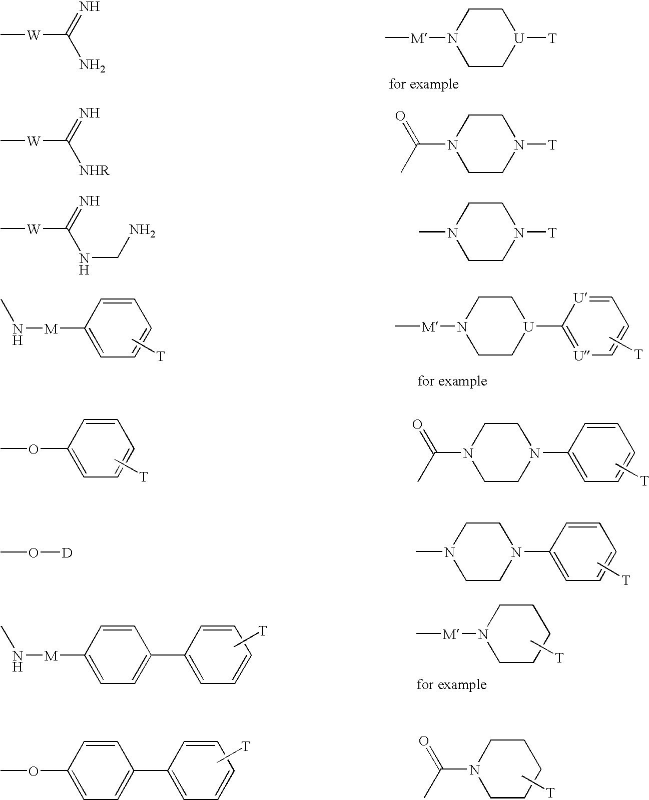

In the above formulae:

W is nothing or —CHR′—S—, where R′ is H or C₁-C₁₀ alkyl;

R is C₁-C₁₀ alkyl, —NHCO—(C₁-C₁₀)alkyl, or C₅-C₁₀ cyclohydrocarbyl (for example aryl, e.g. phenyl or cyclohexyl);

M is M is SO₂, CO, (CH²)_{q}SO₂, (CH₂)_{q}CO, CO(CH₂)_{q}CO [especially CO(CH₂)₂CO] or CHMeCO, where q is 1, 2, 3, or 4;

T is H, C₁-C₁₀ alkyl, C₁-C₁₀ alkoxy, —CHO, —SO₂R, halo (especially F, Cl or Br and most especially F or Cl), nitrile or a residue of a natural or unnatural amino acid (e.g. glycine);

D is a C₁-C₁₆ moiety constituted by alkyl, alkylene, cycloalkyl and/or cycloalkylene residues wherein the alkyl and alkylene residues may be interrupted by, or linked to an adjacent residue by, an —O— linkage or be substituted by —CHO, —SO₂R, or a residue of a natural or unnatural amino add (e.g. glycine), for example D may be methyl or cyclohexyl;

M′ is nothing or an M group; U, U′ and U″ are each independently C or, normally, N and, if C, are optionally substituted by C₁-C₁₀ alkyl or C₁-C₁₀ alkoxy independently of the substitution status of the others of U, U′ and U″; and

alkyl moieties (including the alkyl part of alkoxy) are preferably C₁-C₈ or, more preferably, C₁-C₆ and often have 1, 2, 3 or 4 carbon atoms.

One preferred class of compounds, in particular useful as serine protease inhibitors, is of formula (VI), in which the Q moiety —NH-M-Ph-Ph-T is preferably at the 4-position to —BY¹Y²:

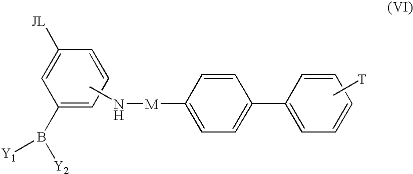

Exemplary compounds of Formula (VI) have the following combinations of T and M groups:

| T | M | T | M | T | M |
|---|---|---|---|---|---|
| 4′-OH | CO | 4′-Me | CO | 4′-N-hexyl | CO |
| 4′-Et | CO | 2′-Me | CO | 2′-F | CO |
| 4′-(2-pyridyl) | CO | 4′-Cl | CO | 4′-Br | CO |
| 4′-(3-pyridyl) | CO | 2′-OMe | CO | 4′-CH₃(CH₂)₇—O | CO |
| 4′-tBu | CO | 4′-Cl | CO | 4′-CH₃(CH₂)₆—O | CO |
| 4′-F | CO | 4′-CN | CO | 4′-CH₃(CH₂)₂O | CO |
| 4′-EtO | CO | 4′-N-Propyl | CO | 4′-CH₃(CH₂)₄—O | CO |
| | | | | 4′-CH₃(CH₂)₃—O | CO |

Q is most preferably H in formula IV compounds.

One advantageous class of compounds, useful especially as serine protease inhibitors, are those, usually of formula I or II and preferably of formulae III, IV or V, in which the or each -LJ group is of the formula

—CR′₂—S-J¹ (especially —CH₂—S-J¹ or CHMe—S-J¹)

where

each R′ is independently H or alkyl and one R′ is preferably H, and J¹ is C(NH)NH₂ (amidino), —NHC(NH)NH₂ (guanidino), —NH₂ (amino), —C(O)NH₂ (carboxamido), —NH₂OH (hydroxylamino) or imidazolyl (of which amidino and guanadino are preferred), or an N-substituted analogue thereof. In which there is an N-substituent selected from alkyl, cycloalkyl and phenyl, where alkyl has from 1 to 8 carbon atoms, and is preferably methyl, and cycloalkyl has from 4 to 8, normally 5 or 6, carbon atoms. One class of such compounds of formula III, IV or V have Q as H or another Q′ group as described below.

Some particularly preferred compounds of the invention, which in particular (but not exclusively) are advantageously used in the inhibition of coagulation serine proteases such as, for example, thrombin, Factor IXa or Factor Xa, are of formula (VII):

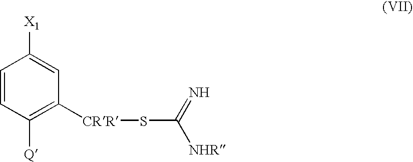

wherein:

X₁ is —B(OR′″)₂, a cyclic boron ester or, less preferably, —NO², where R′″ is preferably H or alkyl;

each R′ is independently H or alkyl and one R′ is preferably H;

R″ is H, alkyl, cycloalkyl or phenyl; and

Q′ is H, alkyl, alkoxyalkyl, alkoxy, aryloxy, aryloxyaryl, wholly or partially hydrogenated analogues of aryl or aryloxyaryl (e.g. cycloalkyloxy) or:

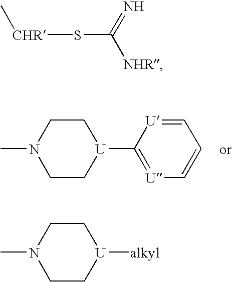

where U, U′ and U″ are each independently C or, normally, N and, if C are optionally substituted by alkyl or alkoxy independently of the substitution status of the others of U, U′ and U″, and R′ and R″ are as defined above. In the compounds of formula (VII), any alkyl or alkoxyalkyl groups have from 1 to 8 carbon atoms, e.g. 1, 2, 3 or 4, and are preferably methyl, and alkoxy has from 1 to 8 carbon atoms, e.g. 1, 2, 3 or 4, and is preferably methoxy; cycloalkyl groups have from 4 to 8 and usually 5 or 6 carbon atoms. Aryl groups are usually phenyl or naphthyl and may be substituted by 1, 2 or 3 substituents selected from halogen, alkyl, hydroxy, alkoxy, thiol, alkylthio, amino, nitrile, carboxy, —CHO, —C(O)alkyl, —SO₂R¹ (where R¹ is as defined previously), and substituted amino (e.g. alkylamino); the aforesaid alkyl groups (including the alkyl part of alkyl-containing moieties) containing from 1 to 10 carbon atoms, e.g 1, 2, 3 or 4, for example methyl or ethyl, and optionally being interrupted by one or a plurality of ether linkages. Needless to say, the compounds may be in the form of salts (including acid addition salts) and/or tautomers of those literally shown, and any boron atom may be in tetrahedral configuration (e.g. with an additional —OH substituent), as is the case with all the compounds of the invention.

In variants of the compounds of formulae (VI) and (VII), any one or two of the three substituent groups on the phenyl ring is replaced by another substituent permitted by the invention. One class of variants comprises compounds in which the phenyl group is replaced by cyclohexyl.

Useful as serine protease inhibitors, especially Factor IXa inhibitors, are compounds of Formula (XIV):

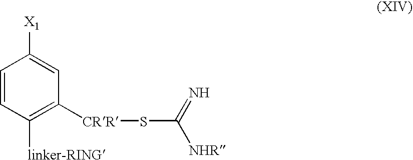

where X₁, R′ and R″ are as defined with reference to Formula (VII), linker is as defined with reference to Formulae (III), (IV) and (V), and RING′ is a mono- or bi-cyclic ring (which may be wholly or partially aromatic) substituted, through a “linker” as previously defined, by a second mono- or bi-cyclic ring (which may be wholly or partially aromatic). Each mono- or bi-cyclic ring may be further substituted by 1, 2 or 3 substituents selected from halogen, alkyl, hydroxy, alkoxy, thiol, alkylthio, amino, nitrile, carboxy, —CHO, —C(O)alkyl, —SO₂R¹ (where R¹ is as defined previously), and substituted amino such as mono- or di-alkyl amino or alkylamido; the aforesaid alkyl groups (including the alkyl part of alkyl-containing moieties) containing from 1 to 10 carbon atoms, e.g. 1 to 4, for example methyl or ethyl, and optionally being interrupted by one or a plurality of ether linkages. Normally each ring is not further substituted or is substituted by a single further substituent. The mono- or bi-cyclic rings are preferably phenyl or a wholly or partially hydrogenated phenyl analogue; the second of the mono- or bi-cyclic rings is preferably attached to the first at the 4-position to where the first is bonded to the remainder of the molecule. Preferably, each linker independently is a bond, —O— or —NH—, of which a bond and, particularly, —O— are preferred. An exemplary linker-RING′ moiety is 4-PhO-PhO, where each phenyl is unsubstituted or substituted as previously described.

Also useful, especially for inhibiting urokinase, are isomers of the compounds of Formula (VII) in which the -LJ group is at the 4-position to Xₗ:

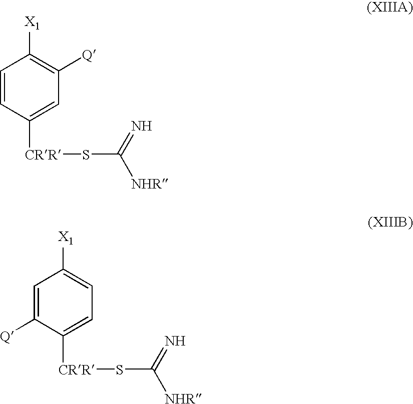

The symbols in Formulae (XIIIA) and (XIIIB) have the same meaning as in Formula (VII). In some preferred Formula (XIII) compounds, Q′ is H.

In variants of the Formula (XIII) and (XIV) compounds, any one or two of the three substituent groups on the benzene ring is replaced by another substituent permitted by the invention, for example another -LJ group and or another Q than those illustrated is used. One class of variants comprises compounds in which the benzene ring is replaced by cyclohexane.

Needless to say, the various preferred compounds described above may be in the form of salts or other derivatives as previously described. In particular, the invention includes prodrugs, for example in which one or more functional groups are protected or derivatised but can be converted in vivo to the functional group, as in the case of protected nitrogens (protected for example by Boc) and derivatised boronate groups convertible in vivo to —B(OH)₂ (which representation includes of course tetrahedral boronate species). By way of example, the invention includes Formula (XIII) and (XIV) compounds which are N-substituted by one or more N-protecting groups.

Synthesis

The synthesis of the compounds of the invention may be performed by using, for example, conventional methods known to the skilled person but, for the reader's assistance, suitable techniques will now be described in the following reaction schemes: The structural formulae shown in the schemes are for the purposes of illustration and, whilst often relating to preferred compounds, the formulae given do not define the scope of the invention. The reaction schemes must therefore be read in conjunction with the description of the invention which appears above.

1. Introduction of Boron to an Aromatic Skeleton.

(i) Halogen-Metal Exchange; ArX+MR→ArM+RX

ArM+BX₃→→ArB(OH)₂

Reactions typically involve an aryl halide, ArX, or a related derivative such as an aryl triflate or trimethylsilyl derivative, (X═I, Br, Cl, OTf, OMs, SiMe₃), and a metal, such as magnesium (Mg, Li, Na, Al, Zn, Si, Hg, Sn . . . ), a Grignard reagent (RM) (e.g. EtMgBr, PhMgBr) or an organolithium (e.g. MeLi, BuLi, PhLi . . . ). Transmetallation is achieved with a borate ester or halide, BX₃, (X=Br, OMe, Oi-Pr, On-Bu, . . . ). These reactions are often carried out at low temperatures, for example −50° C. to −100° C. (e.g. −78° C.) but can also be carried out at room temperature (rt). Often, the initial metallation step requires extreme conditions, e.g. −100° C. or in difficult cases may require heating. A related procedure can be found in Bakker, W. et al. J. Org. Chem. 1994, 59, 972-76. A general review for these metallation procedures and further synthetic manipulations, can be found in Miyaura, N., Adv. Met. Org. Chem., 1998, 6, 187-243. The arylboronic acids can optionally be protected as their ester derivatives by reaction with e.g. the appropriate diol such as pinacol; pinananediol, neopentylglycol or diethanolamine, for example, or attached to a solid support via the acid or ester group for solid state derivatisation. The two examples below are key processes for the formation of polyfunctionalised arylboronic acids synthesised by the inventors.

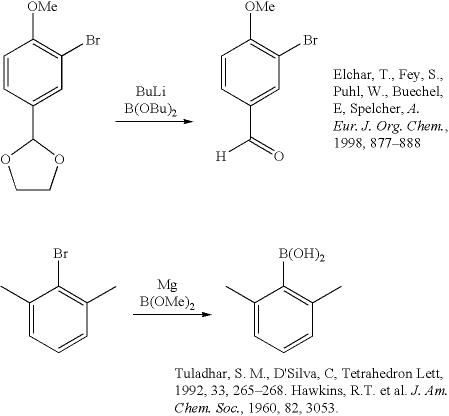

(ii) Direct Metallation: ArH+MR→ArM+RH

ArM+BX₃→→ArB(OH)₂

The aryl group can also be metallated prior to transmetallation with BX₃, typically with a Grignard reagent (RM) (e.g. EtMgBr, PhMgBr) or an organolithium (e.g. MeLi, BuLi, PhLi . . . ) and this is often assisted and directed by the presence of coordinating (e.g. O, S, N, P containing) groups on the aryl ring such as an amide, ether, thioether, amine, heterocyclic group or ester. This protocol has been used in the formation of Losartan, an Angiotension II receptor antagonist—Larsen, R. D. et al., J. Org. Chem., 1994, 59, 6391-6394.

(iii) Boronation of an Aryl Group Under Catalytic Conditions:

HB(OR)₂ or (RO)₂B—B(OR)₂+ArX+catalyst+base→ArB(OR)₂

This route is of increasing popularity for the formation of boronic acid derivatives. Typical catalysts are based on palladium (often of the type PdP₄ or PdP₂Cl₂ where P=phosphine derivative), nickel or platinum complexes and boron derivatives employed include bis(pinacolato) or (neopentylglycolato)-diboron or pinacolborane. Bases include NaOAc, KOAc, carbonates and bicarbonates, hydroxides, amines. Once synthesised, the arylboronic acids can be further derivatised, see below, and optionally deprotected to afford the free boronic add. This method also depends on the availability of suitably substituted arylbromide, iodide and triflate starting materials which can be made via known procedures e.g. aromatic halogenation, metallation-halogenation etc.

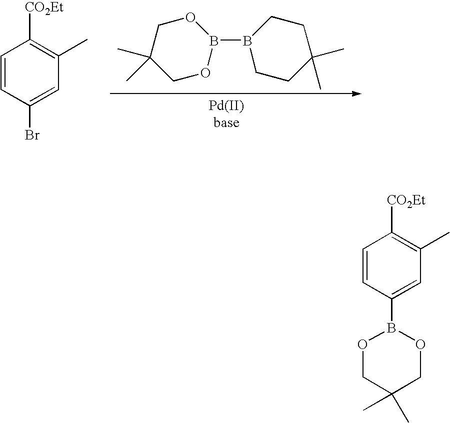

2. Formation of a Benzylhalide or Related Derivative by Aliphatic Halogenations.

ArCH₃+Hal+init.→ArCH₂Hal

The side-chain halogenation of tolyl groups and related derivatives is a convenient method for the formation of benzylhalides and related derivatives. In this scheme, side-chain halogenation of tolyl groups is illustrated with reference to tolyl meta-substituted by —B(OH)₂ but the reaction is in principle applicable to other X groups and other sites of substitution by the X group. These halogenation processes are often radical in nature and involve a halogenating reagent (Hal=elemental halogen or halogenated succinate derivative such as NBS, NCS, NIS), a radical initiator (init. =AIBN, dibenzoylperoxide . . . ), preferably in a polar solvent such as CCl₄ or CHCl₃, often with either heating or a light source such as a 200-500 W bulb. In some cases polyhalogenation can be observed especially when polyfunctionalised arylboronic acid derivatives are employed. The free boronic acid or an ester thereof can be employed.

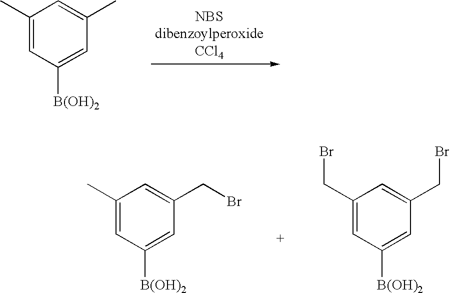

The benzylhalide products are useful intermediates for the formation of further derivatised derivatives, see below.

3. Nucleophilic Substitution of Carboxylic Acid, Carbonyl and Related Derivatives for Further Derivatisation:

(i) Reaction with N, O and S-Based Nucleophiles: RCO₂H→RCOX+Nu→RCONu

Aryl compounds containing carboxylic add groups are useful synthetic intermediates for further synthetic elaboration. In this scheme, the reaction of carboxyl acid groups contained in the side chains of aromatic structures is illustrated with reference to benzene rings para-substituted by boronate groups (e.g. as the acid or an ester) but the reaction is in principle applicable to compounds containing other X groups and other sites of substitution by the X group. Often activation of the carboxylic add is required (to RCOX where X=Cl, Br, I, Oalkyl, Oaryl, succinyl, OC(═O)Oi-Bu etc) prior to reaction with a nucleophile (Nu=amine, alcohol, thiol, etc) with final products including amides, esters, aldehydes, ketones and alcohols. This activation can be effected by the formation of an ester, acyl halide or mixed anhydride derivative or with the use of a typical activating agent such as TBTU, HOBT, DCC, DIEA and procedures and common reagents are described in commercial catalogues such as Novabiochem. O-succinate esters have been described by P. S. Dobbin, R. C. Hider, A. D. Hall, P. D. Taylor, P. Sarpong, 3. B. Porter, G. Xiao and D. van der Helm, J. Med. Chem., 1993, 36, 2448-58 and similar arylboronic acid containing carboxylic acid succinate derivatives were employed by Hamachi, I., Kimura, O., Takeshita, H., and Shinkai, S. Chem. Lett. 1995, 529. The parallel synthesis of amides via activated adds in the presence of scavenger reagents was described by Weidner, J. J., Parlow, J. J., Flynn, D. L., Tetrahedron Lett. 1999, 40, 239-42. A typical reaction scheme is shown below for the purposes of illustration.

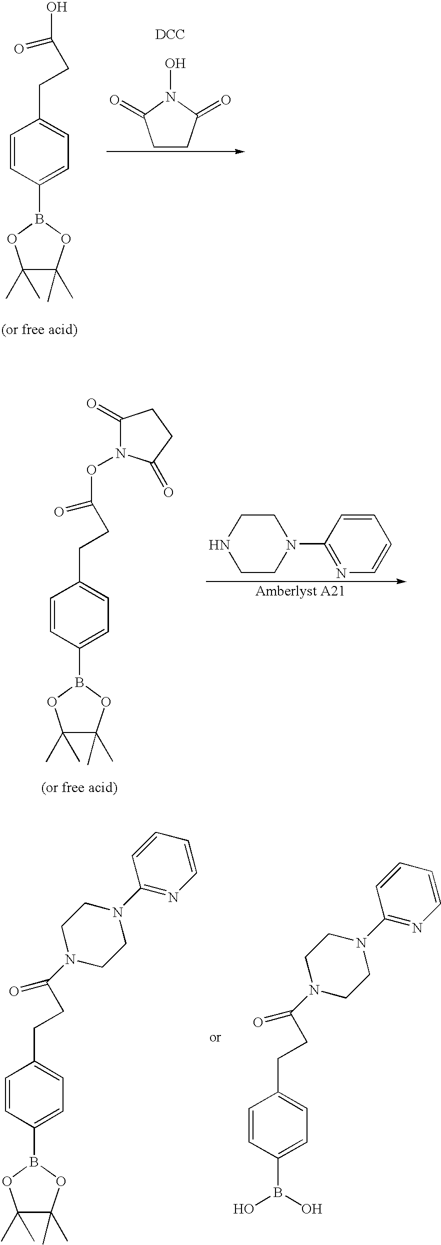

(ii) Reaction with Carbon-Based Nucleophiles

RCOX→RCOR′ or RR′R′COH

RC(═O)H→RR′CHOH

Reactions of carboxylic acid derivatives with carbon-based nucleophiles are well-known and, products range from ketones and aldehydes to alcohols. The addition of an alkyl, alkenyl, allyl, aryllithium or related metallated derivative e.g. malonate, activated ester, Umpolung anion such as dithiane anion, can lead to a secondary alcohol which following activation (e.g. Mitsonobu reaction, conversion to a leaving group-substituted compound, for example halide or tosylate or mesylate) can give an isothiouronium derivative often with a chiral benzylic centre. This reaction is particularly amenable to solid state synthesis, with the boron being linked to the resin (D. G. Hall, J. Tailor, M. Gravel, Angew. Chem. Int. Ed. 1999, 38, 3064.

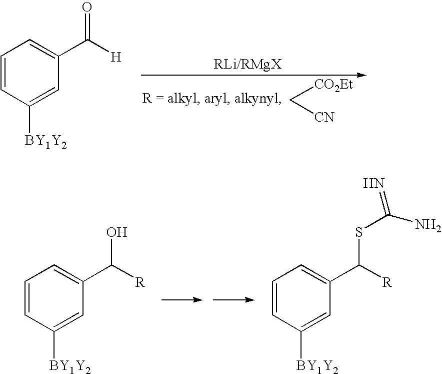

4. Nucleophilic Substitution of a Halide or Related Derivative:

(i) Displacement of an Aliphatic Halide or Related Derivative by a Nucleophile:

RLG+Nu→RNu+LG

This process is very useful for the formation of cyclic compounds having a substituted alkyl side chain, for example substituted boronic acid derivatives. Typically, a nucleophile (Nu=e.g. thiol, thiol derivative, amine or derivative thereof, alcohol, phosphine, metallated aryl, alkyl group, CN, enolate, Umpolung of a carbonyl group) reacts with a benzyl halide or related derivative (leaving group LG=Br, Cl, I, OMs, OTos, etc). These reactions have been used for the synthesis of functionalised isothiouronium, guanidino, thioether, amino boronic acid derivatives using general synthetic protocols (isothiouroniums—Vogel's Book of Practical Organic Chemistry, 5^{th} Edition, B. S. Furniss, A. J. Hannaford, P. W. G. Smith, A. R. Tatchell, P789, Longman Scientific and Technical, N.Y. and guanidines—J. Org. Chem. 1997, 62, 4867-69). In the following scheme, the leaving group is represented by Br and the nucleophile by a thiol or amine (shown as their tautomers). The aryl group on which the aliphatic bromide is substituted is represented by phenyl meta-substituted by a boronate group (shown as the acid or pinacol ester) but the reaction is in principle applicable to compounds containing other X groups and other sites of substitution by the X group.

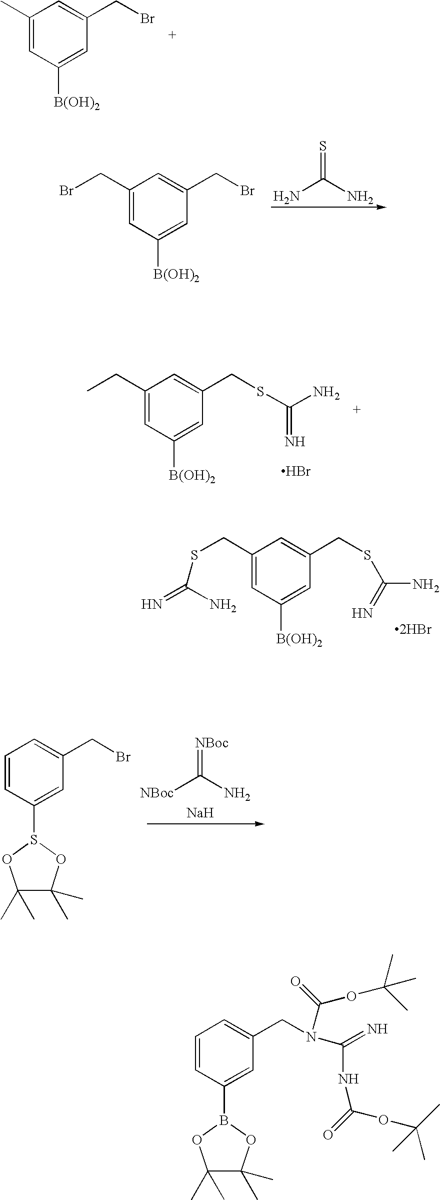

(ii) Nucleophilic Aromatic Substitution:

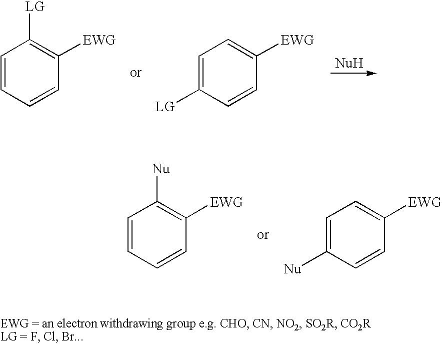

Here, LG is a leaving group directly bound to aryl, typically F, Cl, Br which is suitably activated for a S_{N}Ar type process i.e. where an electron withdrawing group is present on the aryl group e.g. where EWG=CHO, CRO, CO₂R, CN, NO₂, SO₂R, P═O(OR)₂. η⁶-Cr(CO)₃ (Suffert et al. Synlett, 2000, 6, 874-6) or EWG is an aldimine [CH═NR] Cahiez, G et al., Synthesis 1999, 12, 2138-2144. and which is usually either in an ortho or para position relative to X. We have usually chosen EWG to be a group which can be converted into an -LJ group and especially to be —CRO (R=R⁵ as defined above) which can be converted to CR-leaving group for further transformation. Nucleophiles are typically amines, alcohols, nitrile, azide, carbon based anions e.g. activated esters, enolates, malonates, phosphorus containing derivatives and thiols. Following the nucleophilic substitution process, the EWG or nucleophile can be modified e.g. by reduction, c.f. Scheme 5. Modern methods are focusing on the use of metal catalysts, based mainly on palladium, nickel and copper, to promote these reactions, even in the absence of an EWG (Buchwald et al. Angew. Chem. Int. Ed. 1995, 34, 1348. Hartwig et al. Tetrahedron Lett. 1995, 36, 3609. Beller et al. Tetrahedron Lett. 1997, 38,2073.)

We have modified the procedure of Nijhuis, W. H. N., Verboom, W., and Reinhoudt, Synthesis, 1987, 641-45, for the synthesis of polysubstituted aryl compounds (e.g. arylboronic acids) via the displacement of a fluoro substituent ortho to an aldehyde function.

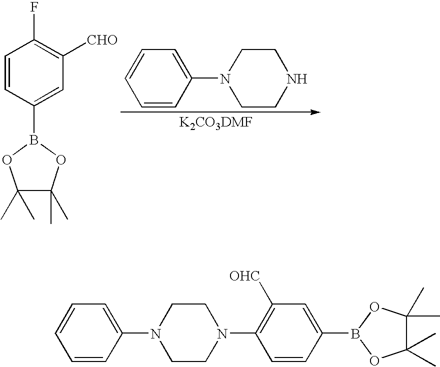

Alternatively, EWG can be converted to Q and the nucleophile can be converted into LJ as below.

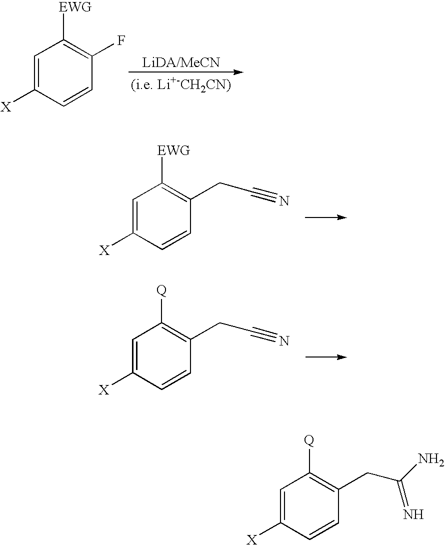

5. Reduction:

(i) Reduction of Acids and Derivatives, Aldehydes, Ketones, Azides and Nitriles:

RCOX→RC(═O)H or RCH₂OH:

RCN→RC(═NH)NH₂ or RCH₂NH₂

RN₃→RNH₂

Here, an aryl compound, for example an arylboronic acid derivative, containing a carbonyl, azide or nitrile group reacts with a reducing agent for example a metal hydride (e.g. LiAlH₄, Dibal, NaBH₄), hydrogen in the presence of a suitable catalyst, or Grignard type or related reagent to afford either an alcohol, amidine or amine derivative respectively, which may be further derivatised according to procedures described herein or known to the skilled person. These synthetic transformations can be employed, typically following activation (e.g. Mitsunobu reaction, conversion to a leaving group-substituted compound, for example halide or tosylate or mesylate), for the formation of a trisubstituted boronic acid isothiouronium derivative or benzamidines (a complementary route to NH₃/HCl or H₂S, MeI methods). The following scheme illustrates the reaction using an aryl boronate and dibal as the reducing agent.

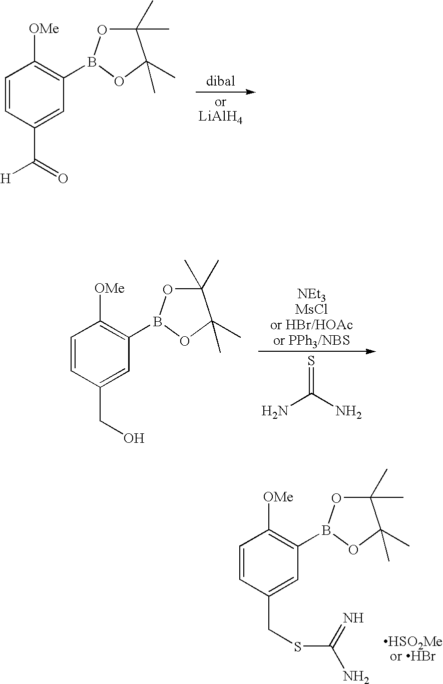

(ii) Reduction of Aryl Boronic Acid Derivatives

The unsaturated rings of unsaturated heterocycles substituted by boronic acid (e.g. as an ester, for example of a diol) may be reduced, preferably in the presence of a metal catalyst such as Adam's catalyst or other platinum, palladium, rhodium or a similar well-established system, under either a hydrogen atmosphere or by transfer hydrogenation. The variety of substituted pyridines and quinolines and related heterocyclic derivatives available render this method attractive for the synthesis of polyfunctional boron containing piperidine and related systems, which can be further modified by the methods described herein. The nitration of an aromatic system (see Scheme 6) can be used to form polyfunctionalised nitroarylboronic acids, which can be reduced to amines under reductive conditions, as above, or by the use of transfer hydrogenation or reductive metal processes (Fe, HCl, Sn, HCl . . . ) The amines can be further functionalised e.g. via diazonium salts (also see Scheme 7).

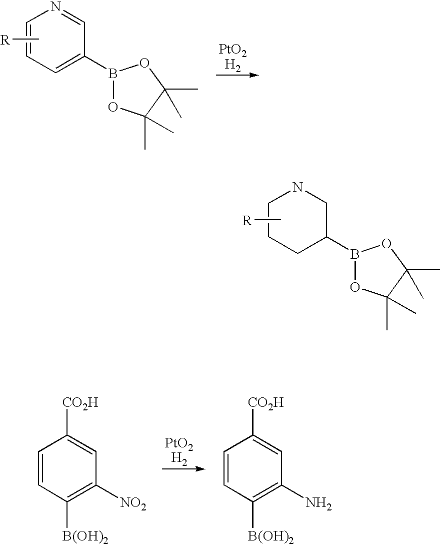

6. Nitration of Aryl Compounds; ArH+NO₂⁺→ArNO₂+H⁺

A variety of methods have been developed for the nitration of aryl groups, including nitric acid-based routes, which have already been used for the formation of trisubstituted arylboronic acids (Soloway et al. J. Am. Chem. Soc. 1959, 3017) as well as nitronium ion-based reagents. These methods are attractive since they form polyfunctionalised nitroaryl compounds, for example nitroarylboronic acid derivatives, which not only have modified electron properties due to the electron withdrawing effect of the nitro group, but also have a nitro group which can be reduced to an amine for further derivatisation or solid phase attachment. Nitration of aromatic compounds is represented in the following scheme by nitration using HNO₃ and H₂SO₄ of arylboronic acids.

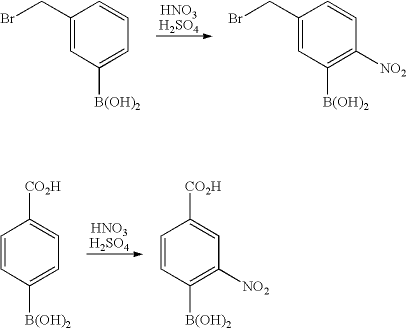

7. Reactions of Amine Containing Aryl Compounds;

e.g.

RNH₂+E⁺→RNHE+H⁺

R₂NH+E⁺→R₂NE+H⁺

Aryl compounds, for example arylboronic adds, containing amino groups can be made from a variety of routes as outlined above and are versatile precursors for other compounds by a host of methods including the nucleophilic attack of the amine on an electrophilic centre such as an aldehyde (also for reductive aminations), acyl derivative, guanidino, cyanate, isocyanate, isothiocyanate, sulphonyl derivative. This route can be employed for the synthesis of amides (see Scheme 3), amines, guanidines, thioureas, isothioureas, carboxamides, sulphonamides and ureas e.g.

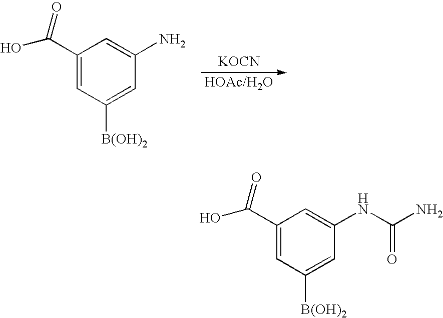

In the above scheme, reaction of an arylamine is represented by reaction of an amino-substituted arylboronic acid to form an amidino group.

8. Carbonylation, Hydroxyalkylation and Halomethylation of Aryl Compounds:

ArH or ArHal+CO→ArCHO

ArH+HCHO→ArCH₂OH+H⁺

ArH+HCHO→ArCH₂OH+HHal→ArCH₂Hal+H₂O

A variety of reagents are known for the carbonylation, hydroalkylation and haloalkylation of aryl systems (e.g. arylboronic acids), notably based on classical Gatterman-Koch, Vilsmeiyer and Reimer Tiemann procedures and modern variants (e.g. metal-catalysed carbonylation of aryliodides and bromides-paper Tsuji, 3. ‘Palladium Reagents and Catalysts’, 3. Wiley and Sons, 1995, chap.4, pp125-290. ISBN 0471 972029; Bohmer, V.; Marschollet, F. and Zetta, L. J. Org. Chem., 1987, 52, 3200-3205; Olah, G. A., Beal., D.a. Yu, S. H.; and Olah, 3. Synth. Commun. 1974, 560-561.). These procedures are especially interesting for the synthesis of polysubstituted arylboronic acid derivatives, which can be further derivatised especially for the synthesis of isothiouronium and guanidine systems. These procedures are represented below by the haloalkylation of an arylboronic acid (which could of course be as an ester):

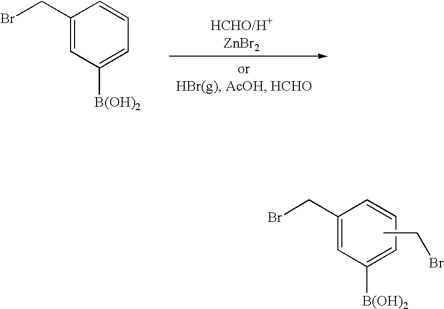

9. Metallation of Arylhalides and Related Derivative's Followed by Reaction with Electrophile:

RHal→RM→RE

These methods comprise metallation of an aryl group (as in Scheme 1) then quenching with e.g. elemental sulphur, or other carbon, nitrogen based reagents (e.g. DMF, CO₂). In the case of the sulphur derivative, the anionic thiophenol derivative can be further functionalised with e.g. an acyl, alkyl, epoxide, guanidine group.

10. Derivatization of amino-carboxylic-phenylboronate.

The carboxylate can be protected by standard protecting groups (see T. Greene, ‘Protecting groups in Organic synthesis, J. Wiley and Sons, 1991, NY, Chichester, Brisbane, toronto.) or by esterification to a resin(see scheme). The nitro group can be reduced to the amino on the resin (by catalytic hydrogenation or sodium thiosulphate) or protected derivative or the amino species can be protected directly. The amino group can then be derivatized (Ar in scheme). The Protection of the carboxylate can be removed, ideally by reduction and cleavage liberating the benzylalcohol, which can be converted to the U species (see below, method 5 or by the intermediacy of a halomethyl derivative).

11. Preparation of Trisubstituted Arylboronates.

The application of the above techniques to the preparation of trisubstituted arylboronates is summarised in the following illustrations:

(1) Route to Trisubstituted boronates (I) and (II)

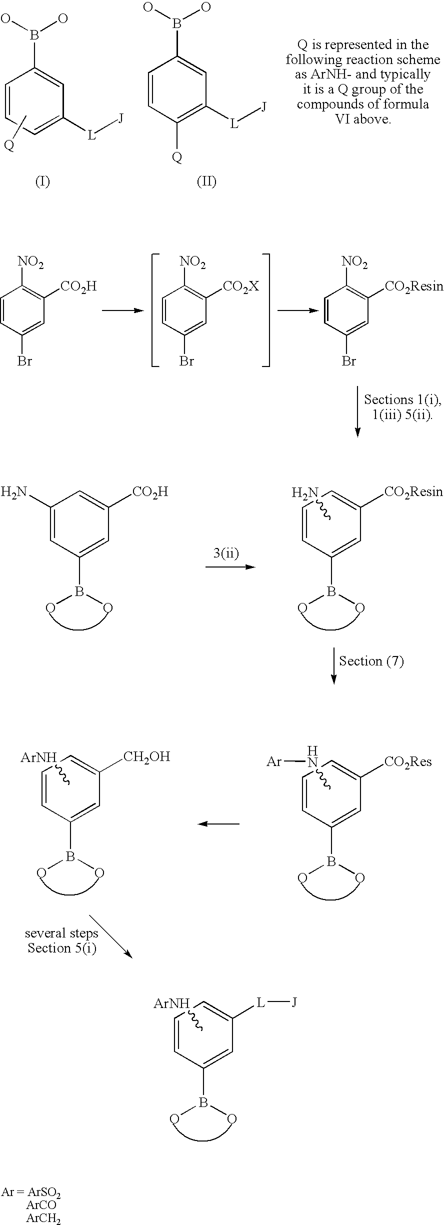

Of the above steps, the reduction of resin-bound aryl nitrate and the cleavage from the resin of the resultant arylamine followed by reduction of the linking carboxylate group are new and included in the invention.

(2) Formation of S or N-Substituted Methylphenylboronic Acids via the Substitution of Leaving Group-Substituted (e.g. Halide or Mesylate) Derivatives by S or N Nucleophiles.

(i) Bromide made from halogenatlon of tolylboronic acid derivatives e.g.:

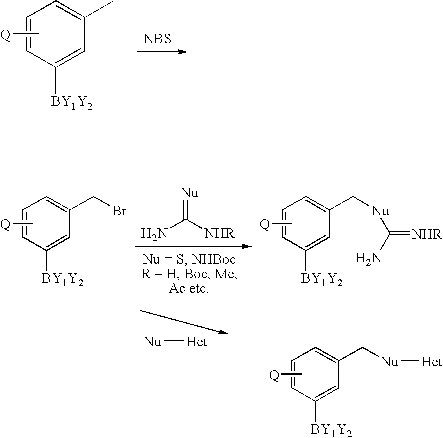

The nucleophilic substitution steps (described more fully in Scheme 3) are novel and included in the invention.

(ii) (a) Leaving group-substituted derivative (e.g. bromide or mesylate) made following reduction or alkylation of carbonyl precursor e.g.

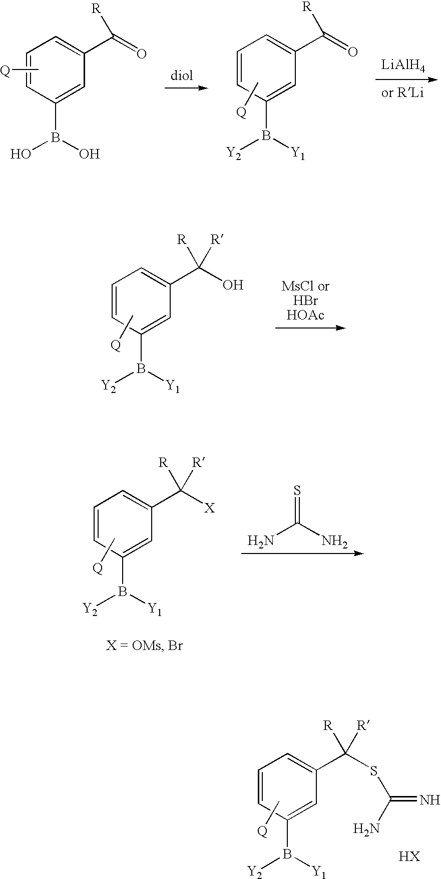

The addition/reduction reactions with LiAlH₄ or R′Li (described in Schemes 3(i) and 5(i)) and the activation reactions with, e.g. MsCl or HBr (also described in Schemes 3(i) and 5(i)), are novel and included in the invention. The products of these reactions are novel intermediates which themselves form an aspect of the invention.

(ii) (b) Leaving group-substituted derivative (e.g. bromide or mesylate) made from polysubstituted arylboronate carbonyl precursor following nucleophilic aromatic substitution reaction on activated arylboronate.

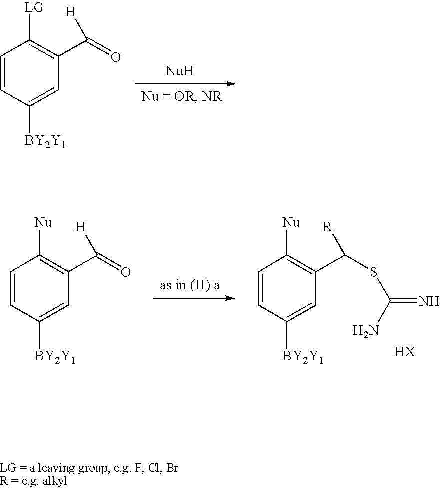

In the above illustration, the aryl group is represented by phenyl meta-substituted by a boronate group but the reaction is in principle applicable to compounds containing other X groups and other sites of substitution by the X group. The substitution reaction on the arylboronate (described in Scheme 4(ii)) and its resultant intermediate are novel and themselves included in the invention.

(iii) Use of a deprotecton strategy enabling further elaboration of polysubstituted boronates e.g.

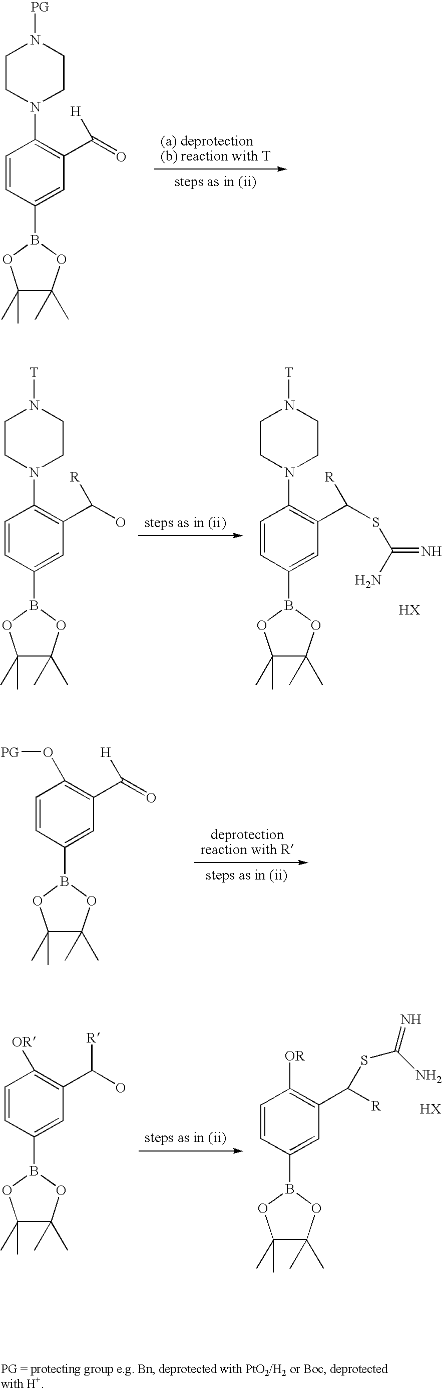

Here, a protecting group is utilized in the starting material, which is then deprotected to yield a common precursor for reaction with T or R¹ groups to yield other derivatives. This is especially desirable for parallel synthesis and protection/deprotection strategies are outlined in T. W. Greene, P. G. M. Wuts, Protective Groups in Organic Synthesis, Wiley, NY, 1991, ISBN 0-471-62301-6.

(iv) ‘One-pot’ reaction without isolation of the leaving group substituted-derivative (.e.g bromide)

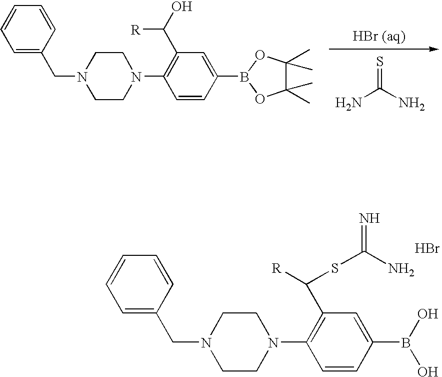

There is literature precedent for the one pot method with alkyl and aryl alcohols (R L Frank, P V Smith, J. Am. Chem. Soc. 1946, 68, 2103) which is here applied to arylboronates.

(3) Amides, e.g. Arylboronate Amides, via Activated Esters e.g.

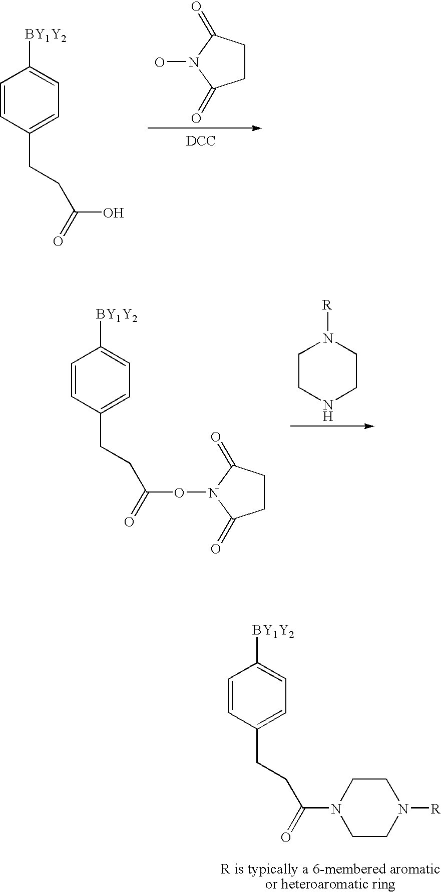

These synthetic steps are described in Scheme 3(i) above.

Novel Methods

The invention includes the methods described herein for synthesising the compounds of the invention and their precursors. Of these methods, the following will be mentioned in particular:

1) Nucleophilic Aromatic Substitution

This method comprises contacting a compound of the formula

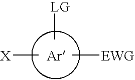

where Ar′ is an aromatic ring, LG is a leaving group, e.g. F, Cl or Br, and EWG is an electron withdrawing group and at the 2- or 4-position to LG, exemplary electron withdrawing groups being CHO, CRO, CO₂R, CN, NO₂, SO₂R, P═O(OR)₂, η⁶-Cr(CO)₃ or [CH═NR] (where R is in principle any organic radical but is most commonly alkyl), and X is as defined above and preferably —BY¹Y²,

with a compound of the formula NuH, where Nu is a nucleophile and especially is the residue of a Q group terminated by a nitrogen atom through which it is joined to Ar′, such as a group of the formula —NH—RING or —NH-M-RING, where M and RING are as defined above. The end product is of the formula:

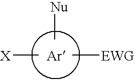

EWG is often a group of the formula [CR⁵R⁶]ₗ₋₁CR⁵(═O), where R⁵ is as defined above and is preferably C₁-C₈ alkyl or hydrogen. It may then be converted to an -LJ group in which L is —CR⁵R⁶—S— using methods 2), 3) and 4) below, and the end product is then suitably purified or isolated and may be formulated into a pharmaceutical or other composition. Another useful EWG group for further transformation is CO₂R. Ar′ is preferably phenyl.

The reaction is suitably carried out in an organic solvent, especially an anhydrous organic solvent, often at elevated temperature. The solvent is desirably polar, for example DMF/DMA. Two illustrative reaction schemes are

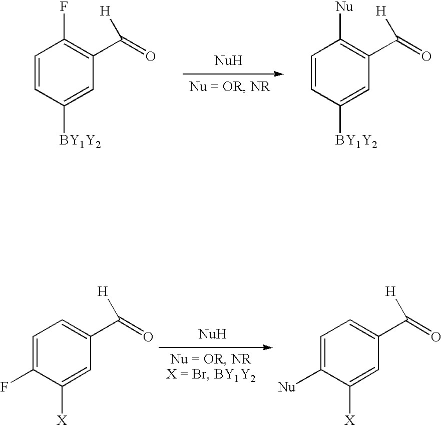

where R is in principle any organic radical, for example OR and NR may be a Q group as described above.

2) Addition to a Carbonyl Function to Give an Aryl Boronate Containing a Secondary or Tertiary Alcohol

This reaction comprises contacting a compound of the formula

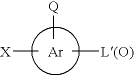

where Ar, Q and X are as defined above and L′ is a chain containing from 1 to 15 carbon atoms including an aldehydic or ketonic carbon atom bonded to the 0 to form a carbonyl group and is preferably [CR⁵R⁶]ₗ₋₁CR⁵(═O), for example formed by performance of a reaction according to preceding method 1),

with a reducing agent or a nucleophile. Suitable reducing agents include metal hydrides (e.g. LiAlH₄, Dibal, NaBH₄) and hydrogen in the presence of a catalyst (e.g. Pd), whilst reagents which may be used as nucleophiles include metallated organic species, for example metallated alkyl, aryl, alkenyl and alkynyl groups as well as metallated forms of their activated derivatives, e.g. malonate, activated ester. The end product is of the formula:

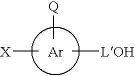

where L′ is a chain containing from 1 to 15, e.g. 1 to 6 and especially 1, 2, 3, or 4, carbon atoms including an alcoholic carbon atom bonded to the OH.

The reaction is suitable performed in an organic solvent, especially a polar solvent, for example THF. The solvent is desirably anhydrous.

Two illustrative reaction schemes are:

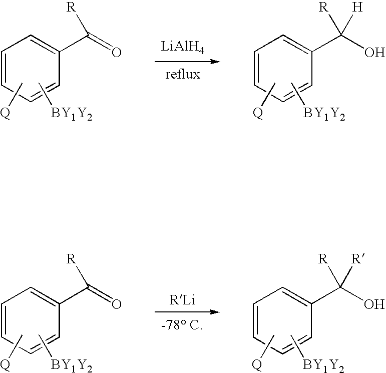

(R′Li may be replaced by another metallated reagent, e.g. R′MgHal).

where R and R′ typically are each independently C₁-C₈ alkyl, C₁-C₈ alkenyl, C₁-C₈ alkynyl or C₆-C₁₀ aryl.

3) Activation of Alcohol to Nucleophilic Substitution

The method comprises contacting a compound which is obtainable by the preceding method 2) and is of the formula

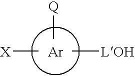

where Ar, Q and X are as defined above and and L′ is a chain containing from 1 to 15, e.g. 1 to 6 and especially 1, 2, 3, or 4, carbon atoms including an alcoholic carbon atom bonded to the OH, -L′-OH preferably being —CR⁵R⁶OH,

with an agent to replace the hydroxy group with a leaving group, for example Br, Cl, I, OMs or OTos (Ms=mesylate, Tos=tosylate). The end product is of the formula:

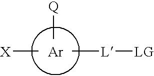

where LG is a leaving group.

The reaction is suitably performed In an organic solvent, especially a non-polar solvent, for example dichloromethane. The solvent is desirably anhydrous. The reaction is typically carried out at low or at room temperature (e.g. from −78° C. to rt).

An illustrative reaction scheme is:

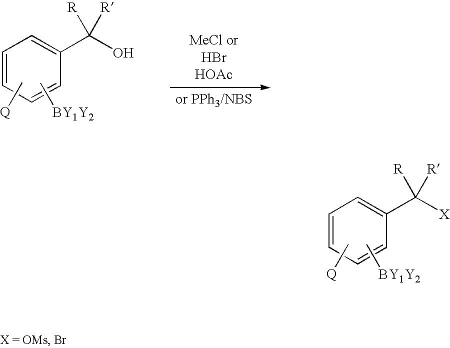

where R and R′ typically are each independently C₁-C₈ alkyl, C₁-C₈ alkenyl, C₁-C₈ alkynyl or C₆-C₁₀ aryl.

4) Nucleophilic Substitution of Leaving-Group Substituted Alkylic Side Chain Moieties

The novel reaction comprises contacting a compound of the formula

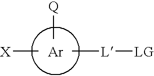

where Ar is as defined above, L′ is a chain containing from 1 to 15 carbon atoms including at least one alkylenic carbon atom substituted by LG, and LG is a leaving group, for example Br, Cl, I, OMs or OTos (Ms=mesylate, Tos=tosylate),

with a nucleophilic reagent, for example a thiol, thiol derivative, amine or derivative thereof, alcohol, phosphine, metallated aryl, alkyl group, CN, enolate, Umpolung of a carbonyl group, which, in the preparation of the compounds of the invention, forms after the reaction a -ZCHR⁷J group or a moiety which can be converted to a -ZCHR⁷J group. L′ is preferably of the formula CR⁵R⁶)ₗ-LG, where R⁵ and R⁶ are as defined above, and is most preferably —CH₂— or —CHR⁵—. Ar is preferably phenyl. X is preferably BY¹Y², especially a boronic acid or boronate ester moiety. The nucleophilic reagent is thiourea in one preferred class of methods.

The end product in the preparation of the compounds of the invention is of the formula:

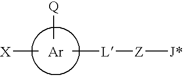

where Z is O, N or S and J* is a J moiety or a group capable of being converted to a J moiety. The end product is suitably purified or isolated and may be formulated into a pharmaceutical or other composition.

The reaction is in practice carried out in the presence of a polar (4a) solvent, for example an alcohol, e.g. a C₁-C₄ alkanol such as methanol or ethanol optionally in admixture with a minor part of water. The reaction temperature is not critical but room temperature or elevated temperatures are typically used. 4b is carried out usually in a non-polar solvent e.g. benzene and typically at room temperature in the presence of a base such as DBU.

An illustrative reaction scheme is shown below:

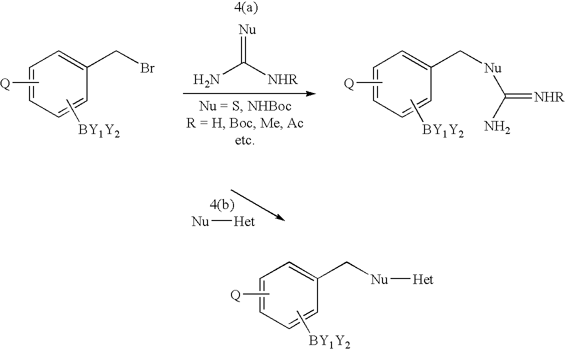

5) Boronation of Resin Bound Aryl Halide

The novel reaction comprises, in a first step, contacting a compound of the formula

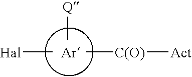

where Ar′ is an aromatic ring, Hal is a Cl, Br or I, Q″ is a Q group or a precursor for a Q group (e.g. NH₂ or NO₂) and Act is —OH or an activating group, (activation of carboxylic acids forms part of the knowledge of any organic chemist and is described above in Synthesis Section Scheme 3(i) by way of example suitable activating groups are an ester-forming group (e.g. Oalkyl, Oaryl), Cl, Br, I, succinyl, OC(═O)Oi-Bu),

with a resin having nucleophilic functional groups, for example Wang resin. Alternatively, the resin can be electrophilic e.g. as with a bromomethyl Wang resin and the product results from the nucleophilic substitution reaction of the halide leaving group by an acid activated as a salt (e.g. with Cs₂CO₃). The end product is of the formula:

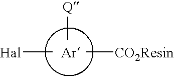

The essential feature of the reaction is its second step, in which the preceding resin-bound aryl halide is converted into a corresponding aryl boronate by halogen-metal exchange or direct metallation as described above in Schemes 1(i). Thus, the aryl halide is reacted with a metallating agent (e.g. a metal, for example Mg, Li, Na, Al, Zn, Si, Hg, Sn, a Grignard reagent (alkylMHal, arylMHal, for example EtMgBr, PhMgBr) or an organolithium compound, for example MeLi, BuLi, PhLi or other alkyl lithium or aryl lithium compound), and the reaction product is contacted with a compound BY¹Y²Y, where Y¹ and Y² are as defined above and Y is halogen or alkoxy, for example Br, OMe, O-iPr, O-n-Bu.

The end product is of the formula:

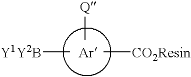

Suitable reaction conditions are described above in Scheme 1(i). An illustrative reaction scheme is shown below:

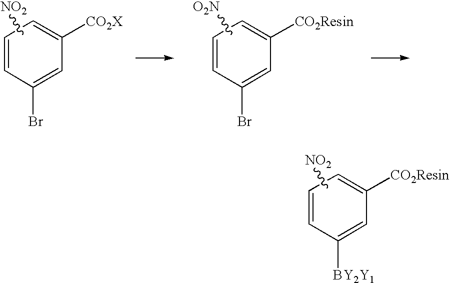

6) Reduction of Resin Bound Nitroaryl Boronate

A resin-bound nitroaryl boronate, obtainable by the preceding method 5),

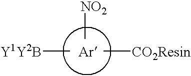

is reacted with a reducing agent, for example a conventional reductant for nitro groups such as hydrogen in the presence of a suitable metal catalyst (e.g Adam's catalyst, platinum, palladium, rhodium), or by the use of transfer hydrogenation or reductive metal processes (e.g. Fe, HCl; Sn, HCl) to afford an amine derivative of the formula:

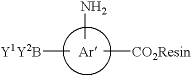

7) Substitution of Amino Group of Resin-Bound Aminoboronate

The amines obtainable by preceding method 6) can be functionalised by reactions described in Synthesis Section Scheme 7. The end product is of the formula:

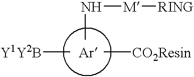

where M′ is as defined above and RING may be replaced by D.

8) Cleavage from Resin and Reduction of Linker Group of Aryl Boronate.

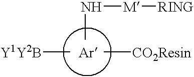

A resin bound aryl boronate obtainable by preceding method 7) and of the formula is contacted with reducing agents as in Synthesis Section Scheme 3(ii) or 5(i) or by displacement by an amine (section 3(i)).

The end product is of the formula:

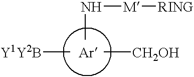

The —CH₂OH group of the end product may be converted to an -LJ group, for example by halogenation followed by nucleophilic substitution as described in Scheme 4 under the heading “Synthesis”. The end product is then suitably purified or isolated and may be formulated into a pharmaceutical or other composition. An illustrative reaction scheme is shown below:

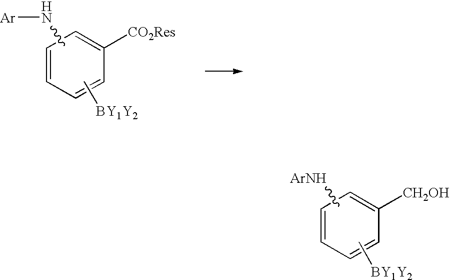

9) “One Pot” Halogenation and Nucleophilic Substitution

The method comprises contacting a compound which in one embodiment comprises a ring or ring system substituted by at least a —BY¹Y² group and a moiety having an alcoholic hydroxy group and which in another embodiment is obtainable by the method 2) and is of the formula

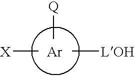

where Ar, Q and X are as defined above and and L′ is a chain containing from 1 to 15, e.g. 1 to 6 and especially 1, 2, 3, or 4, carbon atoms including an alcoholic carbon atom bonded to the OH, -L′-OH preferably being CR⁵R⁶OH,

with aqueous HHal, in particular HBr, and thiourea to give in one step the product of formula:

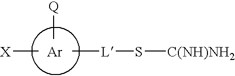

where -L′- is preferably —CR₅R₆—.

An illustrative reaction is:

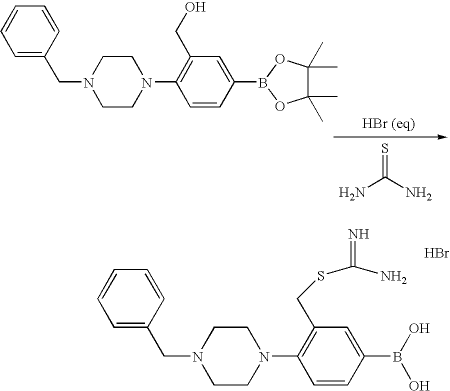

The end product is suitably purified or isolated and may be formulated into a pharmaceutical or other composition.

Intermediates

The invention includes novel compounds useful as intermediates in making the compounds of the invention. Thus, the invention provides compounds of the following formulae

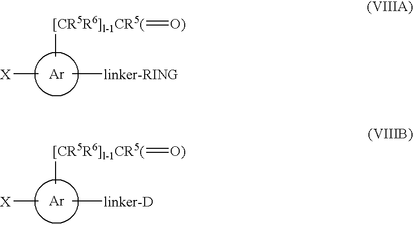

where [CR⁵R⁶]ₗ₋₁CR⁵(═O) is preferably —CHO or C(Me)OH

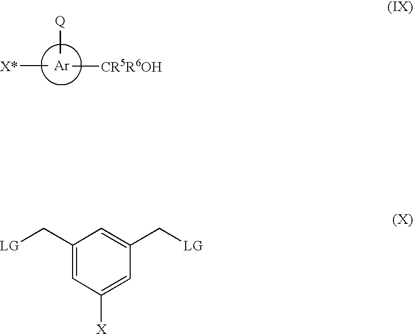

where X* is halogen or an X group.

where LG is a leaving group, for example halogen, mesylate or tosylate.

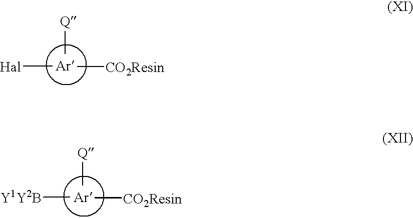

where Q″ is a Q group or a precursor for a Q group, e.g NH₂ or NO₂, and preferably is —NH-M′-RING or —NH-M′-D.

Use

The invention provides a novel class of compounds useful as protease inhibitors and especially as serine protease inhibitors. The compounds find application in the study of proteases, in particular serine proteases, the inhibition of bacterial sporulation, stabilisation of protease-containing detergent compositions, and the inhibition of aberrant or unwanted proteolysis in medical or veterinary applications.

The compounds of the invention are also useful in other applications where their ability to associate with enzymes is of utility, for example affinity chromatography and molecular recognition. The invention also provides compounds which are useful as research tools and intermediates.

A wide range of serine proteases have been implicated in disease states in man and animals, including elastase and cathepsin G, which have been reported to have roles in diseases involving tissue destruction such as emphysema, rheumatoid arthritis, corneal ulcers and glomerular nephritis.

Urokinase (urinary-type plasminogen activator or uPA (International Union of Biochemistry classification number: EC3.4.21.31)) is a serine protease which is highly specific for a single peptide bond in plasminogen. Plasminogen activation (cleavage of this bond by the urokinase enzyme) results in formation of plasmin, a potent general protease.

Many cell types use urokinase as a key initiator of plasmin-mediated proteolytic degradation or modification of extracellular support structures such as extracellular matrix (ECM) and basement membrane (BM). Cells exist, move and interact with each other in tissues and organs within the physical framework provided by ECM and BM. Movement of cells within ECM or across BM requires local proteolytic degradation or modification of the structures and allows cells to invade adjacent areas previously unavailable prior to the degredation or modification.

Cellular invasiveness initiated by urokinase is central to a variety of normal and disease-state physiological processes (Blasi, F., Vassalli, J. D., and Dano, K. J. Cell Biol. 104:801-804, 1987; Dano, K., Anderson, P. A., Grondahl-Hansen, J., Kristensen, P., Nielsen, L. S., and Skriver, L. Adv. Cancer Res. 44:139-266, 1985; Littlefield, B. A. Ann. N.Y. Acad. Sci. 622: 167-175, 1991; Saksela, O., Biochim. Biophys. Acta 823: 35-65, 1985; Testa, J. E. and Quigley, J. P. Cancer Metast. Rev. 9:353-367, 1990). Such processes include, but are not limited to, angiogenesis (neovascularization), bone restructuring, embryo implantation in the uterus, infiltration of immune cells into inflammatory sites, ovulation, spermatogenesis, tissue remodelling during wound repair and organ differentiation, fibrosis, tumour invasion, metastatic spread of tumour cells from primary to secondary sites and tissue destruction in arthritis. Amiloride, for example, a known urokinase inhibitor of only moderate potency, has been reported to inhibit tumour metastasis in vivo (Kellen, J. A., Mirakian, A. Kolin, A. Anticancer Res. 8:1373-1376, 1988) and angiogenesis/capillary network formation in vitro (Alliegro, M. C. and Glaser, B. M. J. Cell Biol. 115[3 Pt 2]: 402a, 1991).

Inhibitors of urokinase, therefore, have mechanism-based anti-angiogenic, anti-arthritic, anti-inflammatory, anti-retinopathic (for anigiogenesis-dependent retinopathies), contraceptive and tumoristatic uses.

In summary, urokinase is a key initiator of extracellular matrix degradation (breakdown of cellular walls) which precedes the cell migration involved in tumor metastasis, angiogenesis and restenosis. An extensive body of evidence supports the validity of urokinase as a target for the development of agents to control the spread and growth of cancer and to prevent re-clogging of blood vessels following vascular surgery such as angioplasty. The compounds of the invention which have an -LJ group at the 4-position to X have beneficial specificity for urokinase, especially when Ar is phenyl or cyclohexyl, especially the compounds of Formula (XIIIA) or (XIIIB).

Serine and other proteases of bacterial origin may cause tissue damage which results from infection. Broad spectrum inhibitors, effective against a number of proteases, are indicated as useful for controlling proteases of bacterial and other pathogenic or parasitic origin.

The invention provides compounds which are particularly potent as inhibitors of trypsin-like enzymes, a group of proteases which hydrolyse peptide bonds at basic residues liberating a C-terminal arginyl or lysyl residue. Among these are the enzymes of the blood coagulation and fibrinolytic system required for haemostasis. They include factors II, IX, X, VII, IXa, XII, thrombin, kallikrein, tissue plasminogen activator and plasmin, as well as urokinase. Enzymes of the complement system, acrosin and pancreatic trypsin are also in this group. The compounds of the invention may therefore be used in vitro for diagnostic and mechanistic studies of trypsin-like enzymes. Furthermore, because of their inhibitory action they are indicated for use in the prevention or treatment of diseases caused by an excess of an enzyme.

The invention therefore provides compounds which have potential for controlling haemostasis and especially for inhibiting coagulation, for example in myocardial infarction. The invention further provides compounds which have potential for inhibiting urokinase and thus controlling tumour metastasis, angiogenesis and restenosis; such anti-angiogenic activity is itself beneficial in controlling metastasis. The medical use of the compounds may be prophylactic as well as therapeutic.

Those compounds of the invention which are beneficially used to inhibit coagulation serine proteases, for example thrombin, factor IXa or factor Xa, have antithrombogenic properties and may be employed when an anti-thrombogenic agent is needed. They are thus indicated in the treatment or prophylaxis of thrombosis and hypercoagulability in blood and tissues of animals including man.

It is known that hypercoagulability may lead to thrombo-embolic diseases. Conditions associated with hypercoagulability and thrombo-embolic diseases which may be mentioned include activated protein C resistance, such as the factor V-mutation (factor V Leiden), and inherited or acquired deficiencies in antithrombin III, protein C, protein S, heparin cofactor II. Other conditions known to be associated with hypercoagulability and thrombo-embolic disease include circulating antiphospholipid antibodies (Lupus anticoagulant), homocysteinemi, heparin induced thrombocytopenia and defects in fibrinolysis. The coagulation serine protease inhibitors of the invention are thus indicated both in the therapeutic and/or prophylactic treatment of these conditions.

Particular disease states which may be mentioned include the therapeutic and/or prophylactic treatment of venous thrombosis and pulmonary embolism, arterial thrombosis (eg in myocardial infarction, unstable angina, thrombosis-based stroke and peripheral arterial thrombosis) and systemic embolism usually from the atrium during arterial fibrillation or from the left ventricle after transmural myocardial infarction.

The thrombin inhibitors of the invention are further indicated in the treatment of conditions where there is an undesirable excess of thrombin without signs of hypercoagulability, for example in neurodegenerative diseases such as Alzheimer's disease. In addition to its effects on the coagulation process, thrombin is known to activate a large number of cells (such as neutrophils, fibroblasts, endothelial cells and smooth muscle cells). Therefore, the compounds of the invention may also be useful for the therapeutic and/or prophylactic treatment of idiopathic and adult respiratory distress syndrome, pulmonary fibrosis following treatment with radiation or chemotherapy, septic shock, septicemia, inflammatory responses, which include, but are not limited to, edema, acute or chronic atherosclerosis such as coronary arterial disease, cerebral arterial disease, peripheral arterial disease, reperfusion damage, and restenosis after percutaneous trans-luminal angioplasty (PTA).

Moreover, the coagulation enzyme inhibitors of the invention are expected to have utility in prophylaxis of re-occlusion (ie thrombosis) after thrombolysis, percutaneous trans-luminal angioplasty (PTA) and coronary bypass operations; the prevention of re-thrombosis after microsurgery and vascular surgery in general. Further indications include the therapeutic and/or prophylactic treatment of disseminated intravascular coagulation caused by bacteria, multiple trauma, intoxication or any other mechanism; anticoagulant treatment when blood is in contact with foreign surfaces in the body such as vascular grafts, vascular stents, vascular catheters, mechanical and biological prosthetic valves or any other medical device; and anticoagulant treatment when blood is in contact with medical devices outside the body such as during cardiovascular surgery using a heart-lung machine or in haemodialysis.

Compounds of the invention that inhibit trypsin and/or thrombin may also be useful in the treatment of pancreatitis.

According to a further aspect of the present invention, there is provided a method of treatment of a condition where inhibition of thrombin is required which method comprises administration of a therapeutically effective amount of a compound of the invention, or a pharmaceutically acceptable salt thereof, to a person suffering from, or susceptible to such a condition.

The compounds of the invention will normally be administered orally, intravenously, subcutaneously, buccally, rectally, dermally, nasally, tracheally, bronchially, by any other parenteral route, as an oral or nasal spray or via inhalation, The compounds may be administered in the form of pharmaceutical preparations comprising prodrug or active compound either as a free compound or, for example, a pharmaceutically acceptable non-toxic organic or inorganic acid or base addition salt, in a pharmaceutically acceptable dosage form. Depending upon the disorder and patient to be treated and the route of administration, the compositions may be administered at varying doses.

The coagulation enzyme inhibitors of the invention may also be combined and/or co-administered with any antithrombotic agent with a different mechanism of action, such as the antiplatelet agents acetylsalicylic acid, ticlopidine, clopidogrel, thromboxane receptor and/or synthetase inhibitors, fibrinogen receptor antagonists, prostacyclin mimetics and phosphodiesterase inhibitors and ADP-receptor (P.sub.2 T) antagonists.

The coagulation enzyme inhibitors of the invention may further be combined and/or co-administered with thrombolytics such as tissue plasminogen activator (natural, recombinant or modified), streptokinase, urokinase, prourokinase, anisoylated plasminogen-streptokinase activator complex (APSAC), animal salivary gland plasminogen activators, and the like, in the treatment of thrombotic diseases, in particular myocardial infarction.

Typically, therefore, the pharmaceutical compounds of the invention may be administered orally or parenterally (“parenterally” as used herein, refers to modes of administration which include intravenous, intramuscular, intraperitoneal, intrasternal, subcutaneous and intraarticular injection and infusion.) to a host to obtain an protease-inhibitory effect. In the case of larger animals, such as humans, the compounds may be administered alone or as compositions in combination with pharmaceutically acceptable diluents, excipients or carriers.

Actual dosage levels of active ingredients in the pharmaceutical compositions of this invention may be varied so as to obtain an amount of the active compound(s) that is effective to achieve the desired therapeutic response for a particular patient, compositions, and mode of administration. The selected dosage level will depend upon the activity of the particular compound, the route of administration, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

Envisaged suitable daily doses of the compounds of the invention in therapeutic treatment of humans are about 0.001-100 mg/kg body weight at peroral administration and 0.001-50 mg/kg body weight at parenteral administration. A preferred peroral dose of from 0.02 to 15 mg/Kg of body weight is envisaged, and the active compound may be given as a single dose, in multiple doses or as a sustained release formulation. For use with whole blood, from 1 to 10 mg per litre may be provided to prevent coagulation.

According to a further aspect of the invention there is thus provided a pharmaceutical composition including a compound of the invention, in admixture with a pharmaceutically acceptable adjuvant, diluent or carrier.

Pharmaceutical compositions of this invention for parenteral injection suitably comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants.

These compositions may also contain adjuvants such as preservative, wetting agents, emulsifying agents and dispersing agents. Prevention of the action of microorganisms may be ensured by the inclusion of various antibacterial and antifungal agents, for example, paraben, chlorobutanol or phenol sorbic acid. It may also be desirable to include isotonic agents such as sugars or sodium chloride, for example. Prolonged absorption of the injectable pharmaceutical form may be brought about by the inclusion of agents (for example aluminium monostearate and gelatin) which delay absorption.

In some cases, in order to prolong the effect of the drug, it is desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are suitably made by forming microencapsule matrices of the drug in biodegradable polymers, for example polylactide-polyglycolide. Depending upon the ratio of drug to polymer and the nature of the particular polymer employed, the rate of drug release can be controlled. Examples of other biodegradable polymers include polyorthoesters) and poly(anhydrides). Depot injectable formulations may also prepared by entrapping the drug in liposomes or microemulsions which are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use.

Solid dosage forms for oral administration include capsules, tablets, pills, powders and granules. In such solid dosage forms, the active compound is typically mixed with at least one inert, pharmaceutically acceptable excipient or carrier such as sodium citrate or dicalcium phosphate and/or one or more: a) fillers or extenders such as starches, lactose, sucrose, glucose, mannitol and silicic acid; b) binders such as carboxymethylcellulose, alginates, gelatin, polyvinylpyrrolidone, sucrose and acacia; c) humectants such as glycerol; d) disintegrating agents such as agar-agar, calcium carbonate, potato or tapioca starch, alginic acid, certain silicates and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay and i) lubricants such as talc, calcium stearate, magnesium stearate, solid polyethylene glycols, sodium lauryl sulfate and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycol, for example.

The solid dosage forms of tablets, dragees, capsules, pills, and granules can be prepared with coatings and shells such as enteric coatings and other coatings well known in the pharmaceutical formulating art. They may optionally contain opacifying agents and may also be of a composition such that they release the active ingredient(s) only, or preferentially, in a certain part of the intestinal tract, and/or in delayed fashion. Examples of embedding compositions which can be used include polymeric substances and waxes.

The active compounds may also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan and mixtures thereof. Besides inert diluents, the oral compositions may also include adjuvants such as wetting agents, emulsifying and suspending agents, sweetening, flavoring and perfuming agents. Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar, and tragacanth and mixtures thereof.

Compositions for rectal or vaginal administration are preferably suppositories which can be prepared by mixing the compounds of this invention with suitable non-irritating excipients or carriers such as cocoa butter, polyethylene glycol or a suppository wax which are solid at room temperature but liquid at body temperature and therefore melt in the rectum or vaginal cavity and release the active compound.

Compounds of the present invention can also be administered in the form of liposomes. As is known in the art, liposomes are generally derived from phospholipids or other lipid substances. Liposomes are formed by mono- or multi-lamellar hydrated liquid crystals which are dispersed in an aqueous medium. Any non-toxic, physiologically acceptable and metabolisable lipid capable of forming liposomes can be used. The present compositions in liposome form can contain, in addition to a compound of the present invention, stabilisers, preservatives, excipients and the like. The preferred lipids are the phospholipids and the phosphatidyl cholines (lecithins), both natural and synthetic. Methods to form liposomes are known in the art, for example, Prescott, Ed., Methods in Cell Biology, Volume XIV, Academic Press, New York, N.Y. (1976), p 33 et seq.

Dosage forms for topical administration of a compound of this invention include powders, sprays, ointments and inhalants. The active compound is mixed under sterile conditions with a pharmaceutically acceptable carrier and any needed preservatives, buffers or propellants which may be required. Ophthalmic formulations, eye ointments, powders and solutions are also contemplated as being within the scope of this invention.

Advantageously, the compounds of the invention are orally active, have rapid onset of activity and low toxicity.

The compounds of the invention have the advantage that they may be more efficacious, be less toxic, be longer acting, have a broader range of activity, be more potent, produce fewer side effects, be more easily absorbed than, or that they may have other useful pharmacological properties over, compounds known in the prior art.

Hypothesis for Interaction of Compounds with Target Serine Proteases.

The kinetics of inhibition of serine proteases by the compounds herein indicate that the compounds bind to the active site of the enzyme. Without meaning to be bound by theory, it is believed that the compounds may then form a set of hydrogen bonding and/or covalent interactions, as seen with peptide boronates and serine proteases. Supposed interactions of illustrative compounds with the active site of thrombin are shown below:

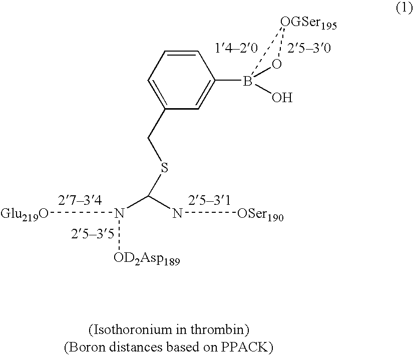

It can be seen from scheme (1) above how Y¹ and Y² (—OH) act as hydrogen bond acceptors whereas, in peptide boronate inhibitors of serine proteases, the boron of a boronate group is considered to act as an electron acceptor.

### EXAMPLES

### Example 1

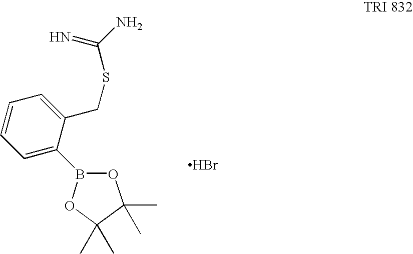

This was synthesised from 2-bromomethylbenzeneboronic acid pinacol ester (297 mg, 1 mmol) and thiourea (76 mg, 1 mmol) following a known general protocol for the synthesis of S-benzylisothiouronium salts (see TRI 864). Yield 110 mg (59%) of a beige solid. ¹H NMR (DMSO-d₆) δ 1.38 (s, 12H), 4.70 (s, 2H), 7.65-7.70 (m, 3H), 7.80 (d, 1H, J5), 9.05 and 9.18 (2brs, 4H). ¹³C NMR (DMSO-d₆) δ 24.9, 34.9, 84.2, 128.0, 130.2, 131.9, 136.7, 140.9, 169.7. MS (ESI) 293 (MH⁺, 100%) calc 292 for C₁₄H₂₁BN₂O₂S.HBr.

### Example 2

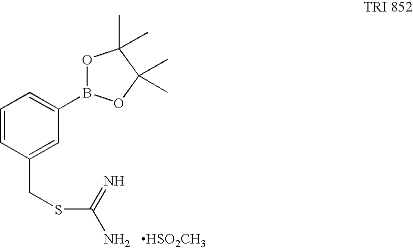

3-hydroxymethylbenzeneboronic acid pinacol ester (228 mg, 0.97 mmol) was combined with excess triethylamine (330 μl, 2.4 mmol) in dichloromethane at room temperature (rt) and methanesulphonyl chloride was added dropwise (95 μl, 1.2 mmol). After 1.5 h stirring, further dichloromethane was added (10 ml) and the organic phase was washed successively with 1N HCl and brine and then dried over Na₂SO₄. Filtration and concentration to dryness yielded an oil which was dissolved in MeOH/H₂O (9 ml/1 ml) and thiourea was added (75 mg, 0.99 mmol). After 1 h reflux, then cooling to rt, the volatiles were removed in vacuo to afford the crude product which was washed with dichloromethane. Yield 80 mg (22%) of a beige solid. ¹H NMR (DMSO-d₆) δ 1.36 (s, 12H), 4.56 (s, 2H), 7.40 (t, 1H, J8), 7.4-7.6 (m, 2H), 7.82 (s, 1H), 9.2 (brs, 3H). ¹³C NMR (DMSO-d₆) δ 25.0, 34.1, 84.1, 128.6, 132.4, 134.2, 135.3, 169.2. MS (ESI) 293 (MH⁺, 100%) calc 292 for C₁₄H₂₁BN₂O₂S.HBr.

### Example 3

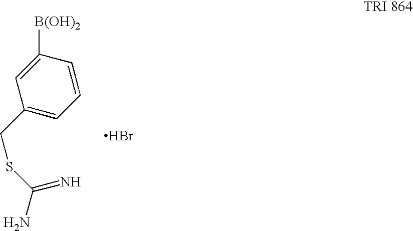

3-bromomethylbenzeneboronic acid (212 mg, 1 mmol) and thiourea (76 mg, 1 mmol) were heated to reflux in MeOH/H₂O (9 ml/1 ml) for 3 h. After cooling to rt, the volatiles were removed in vacuo and the crude product was washed with dichloromethane and acetone. Yield 150 mg (52%) of a beige solid. ¹H NMR (DMSO-d₆) δ 4.48 (s, 2H), 7.34 (t, 1H, J7), 7.44 (d, 1H), 7.73 (d, 1H, J7), 7.79 (s, 1H), 8.99 and 9.19 (2 brs, 4H). ¹H NMR (DMSO-d₆) δ 34.8, 128.2, 130.9, 134.0, 135.1, 169.4. MS (FAB) 481 (M+ bis p-nitrophenol adduct, 100%) calc 210 for C₈H₁₁BN₂O₂S.HBr. Anal calc (found) C, 33.02 (32.94); H, 4.16 (4.11); N, 9.63 (9.31).

### Example 4

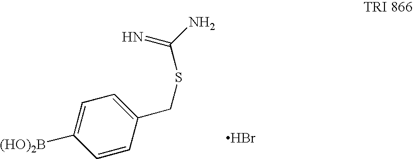

4-bromomethylbenzeneboronic acid (216 mg, 1 mmol) and thiourea (78 mg, 1 mmol) were heated to reflux in MeOH/H₂O (9 ml/1 ml) for 3 h. After cooling to rt, the volatiles were removed in vacuo and the crude product was washed with dichloromethane and acetone. Yield 180 mg (62%) of a beige solid. ¹H NMR (DMSO-d₆) δ 4.47 (s, 2H), 7.36 (d, 2H, J8), 7.76 (d, 2H), 8.97 and 9.17 (2brs, 4H). ¹³C NMR (DMSO-d₆) δ 34.6, 128.3, 134.8, 137.0, 169.2. MS (FAB) 481 (M+ bis p-nitrophenol adduct, 100%) calc 210 for C₈H₁₁BN₂O₂S.HBr. Anal calc (found) C, 33.02 (32.83); H, 4.16 (4.04); N, 9.63 (9.28).

### Example 5

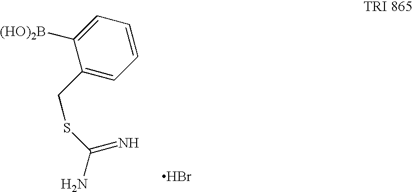

2-bromomethylbenzeneboronic acid (216 mg, 1 mmol) and thiourea (78 mg, 1 mmol) were heated to reflux in MeOH/H₂O (9 ml/1 ml) for 3 h. After cooling to rt, the volatiles were removed in vacuo and the crude product was washed with dichloromethane and acetone. Yield 260 mg (89%) of a beige solid. ¹H NMR (DMSO-d₆) δ 3.48 (brs, 2H), 4.98 (s, 2H), 7.63-7.75 (m, 3H), 8.00 (d, 1H, J7), 9.27 and 9.45 (2brs, 4H). ¹³C NMR (DMSO-d₆) δ 35.0, 127.5, 129.9, 135.4, 139.4, 170.0. MS (FAB) 481 (M+ bis p-nitrophenol adduct, 100%) calc 210 for C₈H₁₁BN₂O₂S.HBr. Anal calc (found) C, 33.02 (32.89); H, 4.16 (4.07); N, 9.63 (9.79).

### Example 6

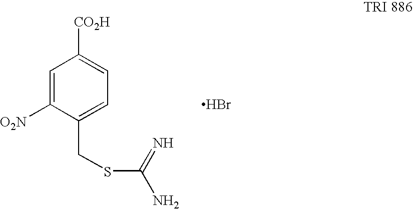

4-(Bromomethyl)-3-nitrobenzoic acid (130 mg, 0.5 mmol) and thiourea (40 mg, 0.53 mmol) in MeOH (10 ml) were reacted as for TRI 852. Yield 105 mg (62%) of a yellow solid. ¹H NMR (DMSO-d₆) δ 4.92 (s, 2H), 7.96 (d, 1H, J8), 8.36 (d, 1H), 8.60 (s, 1H), 9.18 and 9.41 (2brs, 3H). ¹³C NMR (DMSO-d₆) δ 32.2, 126.4, 132.4, 132.6, 134.7, 135.3, 148.3, 165.5, 168.6. MS (ESI) 256 (MH⁺, 100%) calc 255 for C₉H₉N₃O₄S.HBr. Anal calc (found) C, 32.16 (32.34); H, 3.00 (3.08); N, 12.50 (12.60).

### Example 7

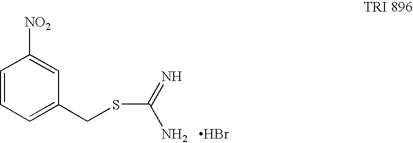

This was synthesised from 3-nitrobenzylbromide (230 mg, 1.06 mmol) and thiourea (90 mg, 1.18 mmol) as for TRI 852. Yield 220 mg (71%) of a white solid. ¹H NMR (DMSO-d₆) δ 4.71 (s, 2H), 7.20 (t, 1H, 77), 7.77 (t, 1H, 78), 7.96 (d, 1H), 8.26 (d, 1H), 8.41 (s, 1H), 9.12 and 9.35 (2brs, 3H). ¹³C NMR (DMSO-d₆) δ 33.4, 123.3, 123.9, 130.7, 135.9, 138.4, 148.2, 168.7. MS (ESI) 212 (MH⁺, 100%) calc 211 for C₈H₁₀BrN₃O₂S.HBr. Anal calc (found) C, 32.89 (32.78); H, 3.45 (3.35); N, 14.38 (14.18).

### Example 8

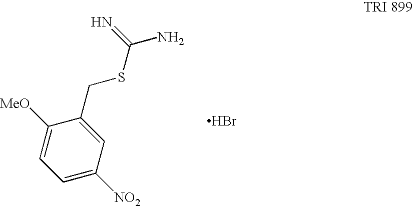

This was made from combining 2-methoxy-5-nitrobenzylbromide (246 mg, 1 mmol) and thiourea (78 mg, 1 mmol) and heating in EtOH (10 ml) at 60° C. for 2 h. After cooling to rt, the volatiles were removed in vacuo to afford the title product. Yield 242 mg (71%) of a beige solid. ¹H NMR (DMSO-d₆) δ 4.06 (s, 3H), 4.50 (s, 2H), 7.37 (d, 1H, J9), 8.34 (d, 1H), 8.44 (s, 1H), 9.13 and 9.31 (2brs, 2H). ¹³C NMR (DMSO-d₆) δ 29.7, 57.2, 112.3, 124.7, 125.9, 126.5, 140.7, 162.7. MS (ESI) 242 (MH⁺, 100%) calc 241 for C₉H₁₁N₃O₃S.HBr. Anal calc (found) C, 33.55 (33.71); H, 3.75 (3.62); N. 13.04 (13.07).

### Example 9

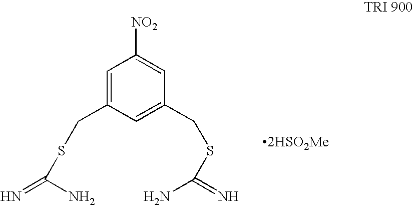

5-Nitro-m-xylene α, α′ diol (183 mg, 1 mmol) was stirred in CH₂Cl₂ (10 ml) with triethylamine (650 μl, 4.8 mmol) and methanesulphonyl chloride (190 μl, 2.4 mmol) was added dropwise.

After stirring at rt for 1.5 h, 1N HCl (5 ml) was added and the organic layer was separated, then washed with concentrated brine. After MgSO₄ drying, the volatiles were removed in vacuo. The resulting pale yellow oil was placed in MeOH (10 ml) with thiourea (76 mg, 1 mmol) and the mixture was refluxed for 1 h. After cooling, the solution was concentrated in vacuo to afford an oil which yielded a white solid after trituration in acetone overnight. Yield 150 mg (33%). ¹H NMR (DMSO-d₆) δ 2.44 (s, 6H), 4.69 (s, 4H), 7.99 (s, 1H), 7.93 (d, 1H, 77), 8.36 (s, 2H), 9.25 and 9.32 (2brs, 6H). ¹³C NMR (DMSO-d₆) δ 33.2, 123.5, 136.0, 138.8, 148.3, 168.8. MS (ESI) 300 (MH⁺, 100%) calc 299 for C₁₀H₁₃N₅O₂S₂.2HSO₂CH₃.

### Example 10

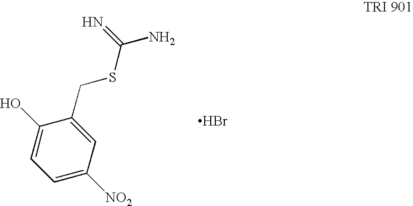

2-hydroxy-5-nitrobenzylbromide (695 mg, 3 mmol) and thiourea (225 mg, 3 mmol) were reacted in EtOH (20 ml) at 60° C. for 2 h. After cooling to rt, the volatiles were removed in vacuo to afford the title product. Yield 750 mg (81%) of a white solid. ¹H NMR (DMSO-d₆) δ 4.46 (s, 2H), 7.07 (d, 1H, J9), 8.14 (d, 1H, J9), 8.34 (s, 1H), 9.05 and 9.23 (2brs, 2H). ¹³C NMR (DMSO-d₆) δ 29.7, 115.9, 122.8, 126.3, 126.7, 139.7, 169.4. MS (ESI) 227 (MH⁺, 100%) calc 226 for C₈H₉N₃O₃S.HBr. Anal calc (found) C, 31.18 (31.27); H, 3.27 (3.38); N, 13.64 (12.86).

### Example 11

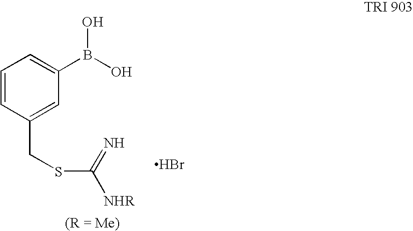

3-bromomethylbenzeneboronic acid (215 mg, 1 mmol) and 1-methyl-2 thiourea (90 mg, 1 mmol) were reacted in MeOH as for TRI 864. Yield 80 mg (26%) of a beige solid. ¹H NMR (DMSO-d₆) δ 2.93 (s, 3H), 4.58 (s, 2H), 7.3-7.5 (m, 2H), 7.8-7.89 (m, 2H), 9.16, 9.47, 9.75 (3brs, 3H). ¹³C NMR (DMSO-d₆) δ 30.8, 35.4, 128.2, 131.1, 133.8, 134.8, 135.1, 165.9. MS (ESI in MeOH) 253 (MH⁺, bis-methanol adduct, 100%) calc 224 for C₉H₁₃BN₂O₂S.HBr.

Other derivatives of TRI 903 were synthesised in a similar manner: R═C(═NH)NH₂ (MS, ESI in MeOH, 281, 100% bis MeOH adduct, 267, mono MeOH adduct, 70%), and R=Ph (MS, ESI in MeOH, 317, 50% bis MeOH adduct, 303, mono MeOH adduct, 100%).

### Example 12

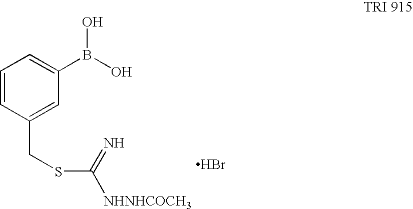

3-bromomethylbenzeneboronic acid (214 mg, 1 mmol) and 1-acetyl-3-thiosemicarbazide (133 mg, 1 mmol) were reacted in MeOH as for TRI 864. Yield 120 mg (35%) of a white solid. ¹H NMR (DMSO-d₆) δ 2.93 (s, 3H), 4.56 (s, 2H), 7.35 (t, 1H, J8), 7.45 (d, 1H), 7.75-7.81 (m, 2H), 9.70, 10.40 (2brs, 3H). MS (ESI, in MeOH) 296 (bis-methanol adduct, 100%), 282 (mono-methanol adduct, 70%), calc 267 for C₁₀H₁₄BN₃O₃S.HBr.

### Example 13

4-(2-carboxyethyl)benzeneboronic acid N-succinate ester, and its pinacol ester (Intermediates)

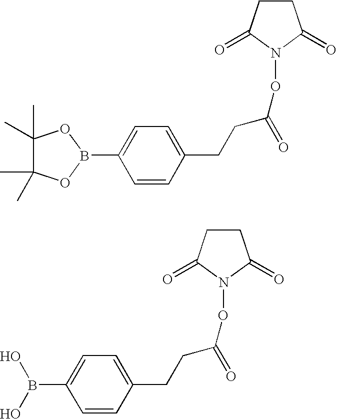

Pinacol ester derivative: 4-(2-carboxyethyl)benzeneboronic acid (1.3 g, 6.7 mmol) was treated with a slight excess of pinacol (790 mg, 6.8 mmol) in anhydrous benzene (50 ml) overnight at reflux temperature in the presence of 3A molecular sieves. After cooling, the solution was transferred by decanting and concentrated in vacuo to yield a white solid (1.22 g, 66%). N-hydroxysuccinimide (250 mg, 0.5 mmol) was added in one go to a DMF solution (20 ml) of the aforementioned 4-(2-carboxyethyl)benzeneboronic acid pinacol ester (550 mg, 2 mmol) under an argon atmosphere. After 10 min, a DMF solution of DCC (412 mg, 2 mmol) was added and the reaction mixture was stirred overnight. The precipitate which had formed was removed by filtration and the filtrate was concentrated in vacuo. Yield 750 mg of a brown solid contaminated with excess N-hydroxysuccinimide. ¹H NMR (CDCl₃) δ 1.33 (s, 12H), 2.71-2.97 (m, 8H), 7.24 (d, 2H, J8), 7.76 (d, 2H). ¹³C NMR (CDCl₃) δ 24.8, 25.6, 30.6, 32.4, 83.7, 127.7, 127.8, 135.2, 167.8, 169.6. The free boronic add derivative was made in an analogous fashion. ¹H NMR (DMSO-d₆) δ 2.81 (s, 4H), 2.89-2.99 (m, 4H), 7.24 (d, 2H, J8), 7.71 (d, 2H), 7.98 ('s, 2H). ¹³C NMR (CDCl₃) δ 24.8, 30.1, 31.9, 134.6, 141.6, 168.7, 170.6. Anal calc (found) C, 53.60 (53.81); H, 4.85 (4.98); N, 4.81 (5.13).

The N-succinate esters are used as the starting material for the procedure of the next example.

### Example 14

General synthetic procedure for the synthesis of 4-(2-carboxyethyl)benzeneboronic acid pinacol ester amide derivatives. Compounds 771, 772, 773, 826, 827, 878, 879, 880 and 926 (intermediates).

4-(2-Carboxyethyl)benzeneboronic add N-succinate ester or pinacol ester (Example 13, 0.5 mmol) is stirred or shaken with one equivalent of an amine or amino acid derivative* in CH₂Cl₂ (3 ml) overnight. The product is either (1) diluted with further CH₂Cl₂ and washed with brine then acid (1N HCl), dried over MgSO₄, filtered and concentrated or (ii) treated with Amberlyst A21 resin for 2 h, then filtered and concentrated. The isolated compounds were estimated to have a purity of ≧80% by. NMR and many were made in parallel. *If the amine or amino acid is an acid salt then excess base, such as NaHCO₃, is added.

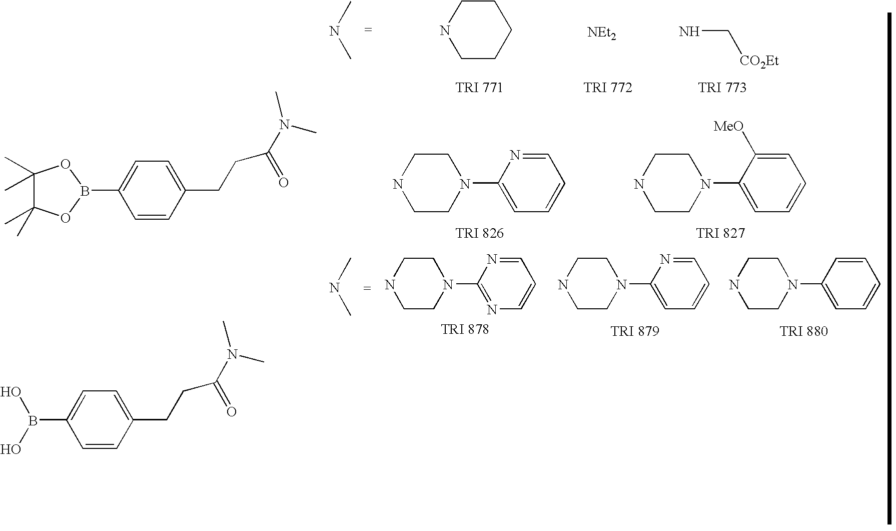

TRI 771 ¹H NMR (CDCl₃) δ 1.33 (s, 12H), 1.4-1.9 (m, 6H), 2.60, 2.97, 3.55, 3.70 (4m, 8H), 7.24 (d, 2H, J8), 7.73 (d, 2H). ¹³C (CDCl₃) δ 24.8, 25.5, 26.4, 29.7, 31.1, 34.9, 42.7, 46.6, 83.7, 127.8, 135.0, 144.9, 170.4. MS (ESI) 709 (2M+Na, 100%), 366 (M+Na, 20%), calc 343 for C₂₀H₃₀BNO₃.

TRI 772

¹H NMR (CDCl₃) δ 0.77 (t, 2H, J8), 1.27 (s, 12H), 2.52 (t, 2H, 78), 2.91 (t, 2H, J8), 3.14 (q, 2H, J7), 3.30 (q, 2H), 7.17 (d, 2H, J7), 7.66 (d, 2H). ¹³C (CDCl₃) δ 13.1, 13.8, 24.8, 29.7, 31.8, 41.4, 41.9, 83.7, 127.9, 135.0, 145.0, 171.1.

TRI 773

¹H NMR (CDCl₃) δ 1.20 (t, 3H, 77), 1.26 (s, 12H), 2.47 (t, 2H, J7), 2.95 (t, 2H), 3.92 (s, 2H), 4.13 (q, 2H, J7), 6.01 (brs, 1H), 7.14 (d, 2H, J8), 7.66 (d, 2H). ¹³C (CDCl₃) δ 13.1, 24.6, 30.6, 36.8, 40.4, 60.5, 82.7, 126.7, 134.0, 143.0, 168.9, 171.0. MS (ESI) 745 (2M+Na, 40%), 384 (M+Na, 100%), calc 361 for C₁₉H₂₈BNO₅.

TRI 826

¹H NMR (CDCl₃) 1.25 (s, 12H), 2.59 (m, 2H), 2.87-94 (m, 2H), 3.41-3.67 (m, 8H), 6.57 (2h, t, J 8), 7.18 (t, 2H, J7), 7.55 (m, 2H), 7.67 (d, 2H, J7), 8.11 (m, 2H). ¹³C (CDCl₃) δ 25.3, 34.3, 35.3, 41.6, 45.1, 45.6, 84.1, 107.8, 114.2, 128.3, 135.5, 138.1, 144.9, 148.4, 159.4, 171.2. MS (ESI) 843 (2 MH+, 40%), 422 (MH⁺, 100%), calc 421 for C₂₄H₃₂BN₃O₃.

TRI 827

¹H NMR (CDCl₃) 1.26 (s, 12H), 2.59-2.96 (m, 4H), 3.45-3.73 (m, 4H), 3.79 (s, 3H), 6.80-6.86 (m, 4H), 7.18 (d, 2H, J7), 7.67 (d, 1H). ¹³C (CDCl₃) δ 25.7, 30.1, 35.2, 42.2, 45.3, 50.0, 55.8, 84.0, 111.6, 118.8, 121.4, 123.8, 128.2, 135.2, 141.0, 144.2, 152.6, 170.9. MS (ESI) 451 (MH⁺, 100%), calc 450 for C₂₆H₃₅BN₂O₄.

TRI 878

¹H NMR (CDCl₃) 2.60 (t, 2H, J7), 2.92 (m, 2H), 3.37 (m, 2H), 3.60-3.70 (m, 6H), 6.44 (dd, 1H, J8, J5), 7.85 (d, 2H, J7), 8.22 (d, 2H, J7). ¹³C (CDCl₃) δ 30.5, 34.2, 40.4, 42.1, 42.5, 42.6, 109.4, 126.6, 133.3, 156.7, 160.4, 170.0.

TRI 879

¹H NMR (CDCl₃) 2.60 (d, 2H, J7), 2.92 (m, 2H), 3.37 (m, 2H), 3.60-3.70 (m, 6H), 6.44 (dd, 1H, J8, J5), 7.85 (d, 2H, J7), 8.22 (d, 2H, 77). ¹³C (CDCl₃): δ 30.5, 34.2, 40.4, 42.1, 42.5, 42.6, 109.4, 126.6, 133.3, 156.7, 160.4, 170.0.

TRI 880

¹H NMR (CDCl₃) 2.62 (d, 2H, J7), 2.88-2.98 (m, 6H), 3.47 (m, 2H), 3.70 (m, 2H), 6.78-6.83 (m, 2H), 7.15-7.28 (m, 5H), 7.94 (d, 2H, J7). ¹³C (CDCl₃) δ 32.1, 34.3, 35.4, 42.0, 44.6, 49.6, 117.0, 117.2, 120.9, 128.3, 128.5, 135.3, 151.3, 171.3.

### Example 15

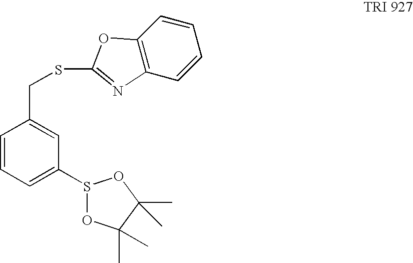

3-bromomethylbenzeneboronic acid pinacol ester (150 mg, 0.5 mmol), methylthiobenzoxazole (75 mg, 0.5 mmol) and DBU (80 μl) were stirred in benzene overnight. After filtration, the filtrate was diluted with further benzene, washed with 1N NaOH, then dried (MgSO₄). The solvents were concentrated in vacuo. Yield 88 mg (48%) of a beige solid. ¹H NMR (CDCl₃) 1.33 (s, 12H), 4.56 (s, 2H), 7.20-7.26 (m, 3H), 7.33 (m, 1H), 7.41 (d, 1H, J8), 7.60 (m, 2H), 7.74 (d, 1H, J7), 7.89 (s, 1H). ¹³C (CDCl₃) δ 24.6, 33.4, 83.9, 109.9, 118.5, 123.9, 124.2, 128.8, 130.9, 134.8, 135.0, 135.4, 141.9, 164.5. MS (ESI) 369 (M+2H+, 100%), calc 367 for C₂₀H₂₂BNO₃S.

### Example 16

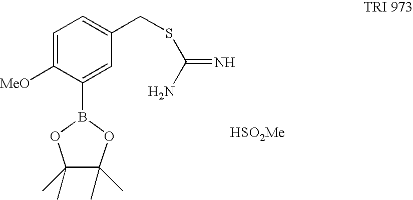

This product was synthesized according to the following multi-step procedure via routes (a) and (b):

a) 3-Formyl-6-methoxyphenylboronic acid pinacol ester, an Intermediate.

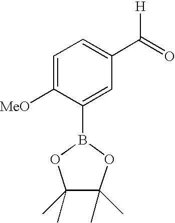

3-formyl-6-methoxyphenylboronic acid (540 mg, 3 mmol) and pinacol (360 mg, mmol) in anhydrous benzene were stirred overnight at reflux in the presence of 3A molecular sieves. After cooling, the solution was filtered and concentrated in vacuo, yielding a virtually quantitative yield of a red solid. ¹H (CDCl₃) δ 1.36 (s, 12H), 3.92 (s, 3H), 6.95 (d, 1H, J8), 7.96 (dd, 1H), 8.20 (d, J2), 9.90 (s, 1H). ¹³C (CDCl₃) δ 24.5, 55.9, 83.9, 110.5, 129.3, 131.0, 139.9, 168.8, 191.0.

b) 2-Methoxy-5-hydroxymethylphenylboronic acid pinacol ester, an Intermediate.

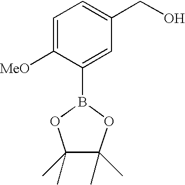

3-Formyl-6-methoxyphenylboronic acid pinacol ester (440 mg, 1.68 mmol) was dissolved in benzene (10 ml) and cooled to 0° C. DIBAL (4 ml, 1M solution in hexanes) was added dropwise and the mixture allowed to warm to rt and stirred overnight. After quenching the reaction with MeOH/benzene (10 ml 1:1 mixture) then 1N HCl (5 ml) the mixture was filtered and extracted with ether and dried over MgSO₄. Concentration yielded an orange oil (250 mg, 95%). ¹H NMR (CDCl₃) δ 1.36 (s, 12H), 3.84 (s, 3H), 4.60 (d, 2H, J7), 6.85 (d, 1H, J7), 7.40 (d, 1H), 7.68 (m, 1H). ¹³C (CDCl₃) δ 24.5, 55.9, 65.0, 83.5, 110.6, 128.8, 130.9, 131.8, 135.9, 163.9.

c) TRI 973. 2-Methoxy-5-hydroxymethylphenylboronic acid pinacol ester (250 mg, 0.95 mmol) was dissolved in dichloromethane (10 ml) and treated sequentially with NEt₃ (320 μl, 2.3 mmol) then methanesulphonyl chloride (100 μl, 1.2 mmol). After 1 h stirring, the mixture was diluted with further dichloromethane (10 ml) and washed with HCl (1N, 10 ml) and brine. After drying over MgSO₄, concentration afforded an oil which was then treated with thiourea (75 mg, 1 mmol) in ethanol (10 ml) at reflux temperature for 2 h. After concentration, a yellow oil/solid was obtained (30 mg, 8%). ¹H NMR (DMSO-d₆) δ 1.27 (s, 12H), 3.73(s, 3H), 4.45(s, 2H), 6.96 (d, 1H, J8), 7.48 (dd, 1H, J8), 7.58 (s, 1H), 8.50, 9.21 (brs, 3H). ¹³C (DMSO-d₆) δ 24.9, 33.9, 55.8, 83.6, 111.4, 126.3, 133.9, 137.5, 162.0, 163.8, 169.5. MS (ESI, MeOH) 348 (M3H++Na, 30%), 247 (M-(S—C(═NH)NH₂), 100%). calc 322 for C₁₅H₂₃BN₂O₃S. FAB 323 (MH⁺, 15%), 247 (M-(S—C(═NH)NH₂), 100%).

### Example 17

### Route to TRI 967 and TRI 968

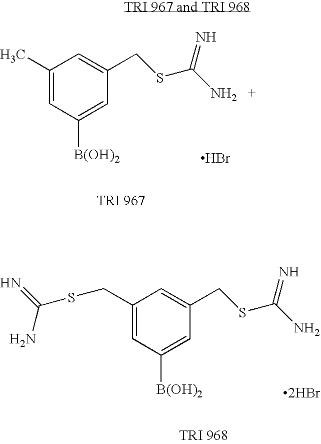

a) 3,5-dibromomethylphenylboronic acid and 3-bromomethyl-5-methylphenyl boronic acid (intermediates)

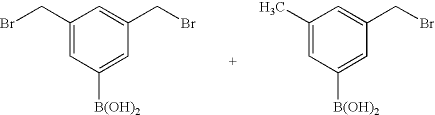

3,5-dimethylphenylboronic acid (1.5 g, 10 mmol) and N-bromosuccinimide (4 g, 22.5 mmol) in anhydrous CCl₄ (100 ml) were illuminated with a 500 W lamp overnight. The resulting mixture was filtered and the white precipitate was air-dried. The latter was washed with acetone (2×50 ml) and the insoluble white residue dried in vacuo before extraction with CH₂Cl₂ and washing with brine and water. After MgSO₄ drying and concentration, NMR analysis showed the presence of starting boronic acid material as well as mono and bis-brominated compounds. Selected ¹H NMR data (DMSO-d₆) δ 2.32 and 2.36 (3H, starting material and monobrominated derivative), 4.73 and 4.77 (2H, mono and bis brominated derivatives), 7.3-8.1 (aromatics). ¹³C (DMSO-d₆) δ 21.3, 35.4, 131.8, 132.5, 132.6, 135.2, 136.9, 131.2.

b) TRI 967 and 968

The mixture from (a) above (110 mg) was heated to reflux for 1 h in EtOH (20 ml) with thiourea (60 mg, excess). After cooling, the volatiles were removed in vacuo affording an oil, which could be triturated with dichloromethane to afford a solid. The dichloromethane soluble fraction was removed by decantation and, after concentration, this was shown to be the starting 3,5-dimethylphenylboronic acid. The solid was dried in vacuo. ¹³C (DMSO-d₆) δ 21.3, 39.2, 131.5, 132.3, 133.9, 134.7, 134.8, 137.2, 169.4. MS (ESI in MeOH) 299 (TRI 968, 15%), 225 (TRI 967, 45%). A series of fractional crystallizations with dichloromethane, then acetone, afforded two fractions—a soluble fraction, the monoisothiouronium compound TRI 967 as an orange oil, which was washed further with acetone to remove excess thiourea: ¹H NMR (DMSO-d₆) δ 2.37 (s, 3H), 4.49 (s, 2H), 7.31, 7.44, 7.65 (3s, 3H), 8.0 (brs, 1H), 9.03 & 9.22 (2brs, 2H). MS (ESI in MeOH) 253 (bis MeOH adduct, 15%), 342 (H₂O adduct, 100%), 239 (MH⁺, MeOH adduct, 30%) calc. 224 for C₉H₁₃BN₂O₂S.HBr and an insoluble white solid which was predominantly the bis-isothiouronium salt TRI 968. The latter was purified by preparative HPLC. ¹H NMR (DMSO-d₆) δ 4.36 (s, 4H), 7.35, 7.65, 8.20 (3s, 3H), 9.08 (brs, 6H). MS (ESI in MeOH) 326 (bis MeOH adduct, 35%), 312 (MH⁺, MeOH adduct, 60%), 237 (-2C(═N)NH₂+Na, 100%) calc 298 for C₁₀H₁₅BN₄O₂S₂.

### Example 18

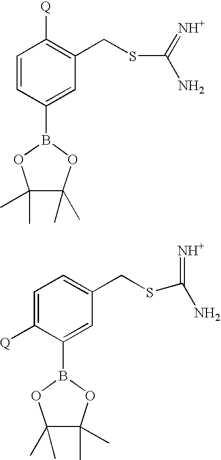

(a) 4-Fluoro-3-formylbenzeneboronic acid, pinacol ester (an Intermediate for Method as via Synthesis Section 4(ii))

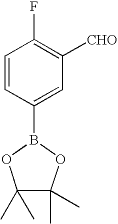

4-Fluoro-3-formylbenzeneboronic add (1.7 g, 10 mmol) was stirred overnight with pinacol (1.24 g, 1.05 mmol) in anhydrous benzene (25 ml) at rt in the presence of molecular sieves. After filtration, the reaction mixture was concentrated in vacuo to afford 2.40 g of a pale yellow solid (97%). ¹H NMR (CDCl₃) δ 1.35 (s, 12H), 7.16 (dd, 1H, J19, J2), 8.02 (m, 1H), 8.33 (dd, 1H, J8, J2). ¹³C (CDCl₃) δ 24.9, 84.4, 115.9(d), 123.7(d), 139 (d), 166(d), 187.3. Anal calc (found) C, 62.44 (62.46), H, 6.45 (6.38).

b) Parallel Synthesis of Piperazine m-Substituted Formyl-Phenylboronic Acids 1a-e and -Bromobenzenes 2a-e (Intermediates).

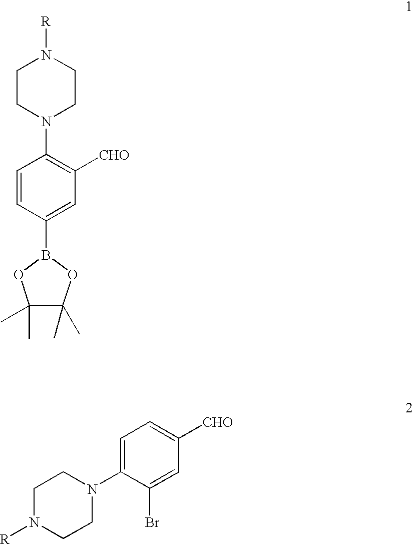

Twelve separate reaction vessels, each containing 4-fluoro-3-formylbenzeneboronic acid pinacol ester (250 mg, 1 mmol) or 3-bromo-4-fluorobenzaldehyde (204 mg, 1 mmol), potassium carbonate (276 mg, 2 mmol) and the appropriate piperazine (1.2 mmol), in anhydrous DMF (10 ml), were placed in parallel (In an Argonaut Firstmate™ apparatus) and the reactions were heated for 24-48 h at 120° C. The course of each reaction was followed by tic. After cooling, each reaction mixture was diluted with ethyl acetate (40 ml) and the organic phase was separated and washed with water and brine before being dried over MgSO₄. Filtration and concentration in vacuo afforded the crude product, which was either used as such or purified by chromatography (SiO₂, typically Et₂O/hexane 60:40 with increasing polarity).

Compounds of the general formula 2 are converted to the boronates via substitution of the arylbromide with BY¹Y² according to section 1 under the heading “Synthesis”. The —CHO groups of the above compounds are converted to —CH₂SC(NH)NH₂ (an -LJ group) via the route described in Example 16 to make TRI 973 and by Scheme 3(ii) and 5(i), under the heading “Synthesis”.

| 1 | R | 2 | R |
|---|---|---|---|
| a | Ph | a | Ph |
| b | o-pyr | b | o-pyr |
| c | o-PhOMe | c | o-PhOMe |
| d | 1-piperonyl | d | 1-piperonyl |
| e | Me | e | Me |
| f | CH₂Ph | f | Ch₂Ph |

1a: ¹H NMR (CDCl₃) δ 1.34 (s, 12H), 3.32 (m, 4H), 3.38 (m, 4H), 6.98-7.30 (m, 6H), 7.87 (dd, 1H, J8, J2), 8.20 (d, 1H, J2), 10.27 (s, 1H). ¹³C NMR (CDCl₃) δ 24.9, 49.4, 53.4, 83.9, 116.3, 117.7, 120.2, 127.7, 129.6, 138.9, 151.1, 191.1. MS (ESI, MeOH) 393 (MH⁺, 40%), 375 (—H₂O, 100%) calc. 392 for C₂₃H₂₉BN₂O₃.

1b: ¹H NMR (CDCl₃) δ 1.34 (s, 12H), 3.27 (m, 4H), 3.74 (m, 4H), 6.98 (m, 2H), 7.09 (d, 1H, J8), 7.50 (m, 1H), 7.70 (m, 1H), 8.20 (d, 1H, J1.5), 10.28 (s, 1H). ¹³C NMR (CDCl₃) δ 24.8, 45.8, 53.1, 83.9, 107.1, 113.4, 128.8, 137.6, 139.2, 141.3, 147.9, 191.1. MS (ESI, MeOH) 390 (M⁺−3H, 100%), 372 (M⁺−H₂O, 60%), 375 (—H₂O, 100%) calc. 393 for C₂₄H₂₉BN₂O₃.

1c: ¹H NMR (CDCl₃) δ 1.34 (s, 12H), 3.31 (m, 8H), 3.87 (s, 3H), 6.88-7.30 (m, 5H), 7.94 (dd, 1H, J8, J2), 8.27 (d, 1H, J2), 10.27 (s, 1H). MS (ESI, MeOH) 467 (M⁺+2Na, 60%), 421 (M+, 100%) calc. 422 for C₂₄H₃₁BN₂O₄.

1d: ¹H NMR (CDCl₃) δ 1.34 (s, 12H), 2.64 (m, 4H), 3.15 (m, 4H), 5.95 (s, 2H), 6.77 (m, 2H), 6.88 (m, 1H), 7.04 (d, 1H, J8), 7.90 (dd, 1H, J8, J2), 8.23 (d, 1H, J2), 10.28 (s, 1H). ¹³C NMR (CDCl₃) δ 24.8, 52.8, 53.4, 62.7, 83.9, 100.9, 107.9, 109.5, 117.7, 122.3, 138.6, 141.2, 147.7, 157.1, 191.2. MS (ESI, MeOH) 451 (M⁺+H₂O+Na, 25%), 451 (M⁺+H₂O, 60%), calc. 436 for C₂₄H₂₉BN₂O₅.

1e: ¹H NMR (CDCl₃) δ 1.33 (s, 12H), 2.38 (s, 3H), 2.64 (m, 4H), 3.18 (m, 4H), 7.05 (d, 1H, J8), 7.92 (dd, 1H, J8, J2), 8.23 (d, 1H, J2), 10.21 (s, 1H). ¹³C NMR (CDCl₃) δ 24.8, 46.1, 49.4, 83.9, 117.7, 127.2, 138.7, 141.2, 156.9, 191.1. MS (ESI, MeOH) 352 (M+Na 100%), 331 (M+, 80%) calc. 330 for C₁₈H₂₇BN₂O₃.

1f: ¹H NMR (CDCl₃) δ 1.27 (s, 12H), 2.59 (m, 4H), 3.42 (m, 4H), 3.53 (s, 2H), 6.96 (d, 1H, J8), 7.24-28 (m, 5H), 7.90 (dd, J8, J2), 8.16 (d, 1H, J2), 10.14 (s, 1H). ¹³C NMR DEPT 135 (CDCl₃) δ 25.4, 118.0, 127.6, 128.7, 129.6, 138.9, 141.6, 191.5 (+ve). 53.3, 53.8, 63.4 (−ve) MS (ESI, MeOH) 407 (MH⁺, 100%), calc. 406 for C₂₁H₃₁BN₂O₃. Anal calc (found) C, 70.94 (71.12), H, 7.69 (7.79), N, 6.89 (6.94).

2a: ¹H NMR (CDCl₃) δ 3.27 (m, 4H), 3.32 (m, 4H), 6.82-7.26 (m, 5H), 7.73 (dd, 1H, J8, J2), 8.02 (d, 1H, J2), 9.86 (s, 1H). ¹³C NMR (CDCl₃) δ 49.7, 51.6, 116.7, 119.5, 120.6, 120.9, 129.6, 130.6, 132.6, 135.9, 151.5, 156.2, 190.3. MS (ESI, MeOH) 345 (M+, 100%), 266 (—Br, 15%) calc. 345 for C₁₇H₁₇BrN₂₀. Anal. Calcd. (found) C, 59.14 (59.18), H, 4.96 (5.06), N, 8.11 (7.96).

2b: ¹H NMR (CDCl₃) δ 3.29 (m, 4H), 3.74 (m, 2H), 6.74 (m, 2H), 7.14 (d, 1H, J8), 7.40 (m, 1H), 7.78 (dd, 1H, J8, J2), 8.08 (d, 1H), 8.10 (m, 1H), 9.85 (s, 1H). ¹³C NMR (CDCl₃) δ 45.8, 51.0, 107.1, 113.2, 119.0, 130.3, 135.4, 147.9, 159.5, 189.9. MS (ESI, MeOH) 346 (M+, 45%), calc. 346 for C₁₇H₁₇BrN₃O.

2c: ¹H NMR (CDCl₃) δ 3.29 (m, 4H), 3.38 (m, 4H), 3.90 (s, 3H), 6.89-7.18 (m, 5H), 7.80 (dd, 1H, J8, J2), 8.01 (d, 1H, J2), 9.80 (s, 1H). ¹³C NMR (CDCl₃) δ 50.7, 51.4, 55.4, 111.2, 118.4, 118.9, 121.0, 123.3, 130.3, 132.0, 135.5, 140.9, 152.3, 189.9. MS (ESI, MeOH) 377 (M++3H, 100%), calc. 375 for C₁₈H₁₉BrN₂O₂.

2d: ¹H NMR (CDCl₃) δ 2.63 (m, 4H), 3.19 (m, 4H), 5.95 (s, 2H), 6.77 (m, 2H), 7.07 (s, 1H), 7.10 (d, 1H, J8), 7.75 (dd, 1H, J8, J2), 8.04 (d, 1H) 9.83 (s, 1H). ¹³C NMR (CDCl₃) δ 52.8, 53.2, 62.7, 100.8, 109.5, 109.6, 118.8, 120.4, 122.3, 130.2, 131.8, 135.1, 146.5, 189.8. MS (ESI, MeOH) 405 (M+hydrate, 95%), calc. 389 for C₁₈H₁₇BrN₂O₃.

2e: ¹H NMR (CDCl₃) δ 2.38 (s, 3H), 2.64 (m, 4H), 3.22 (m, 4H), 7.11 (d, 1H, J8), 7.77 (dd, 1H, J8, J2), 8.06 (d, 1H), 9.85 (s, 1H). ¹³C NMR (CDCl₃) δ 46.0, 51.0, 54.9, 120.5, 130.2, 131.9, 135.5, 189.9. MS (ESI, MeOH) 285 (M+2H, 100%), calc. 283 for C₁₂H₁₉BrN₂O.

2f: ¹H NMR (CDCl₃) δ 2.59 (m, 4H), 3.13 (m, 4H), 3.52 (s, 2H), 7.02 (d, 1H, J8), 7.18-7.29 (m, 5H), 7.69 (dd, 1H, J8, J2), 7.98 (d, 1H, J2), 9.76 (s, 1H). ¹³C NMR (CDCl₃) δ 51.5, 53.4, 63.4, 119.1, 120.8, 127.4, 128.6, 129.6, 130.6, 132.3, 135.9, 138.4, 156.4, 190.3. MS (ESI, MeOH) 361 (M⁺2H⁺, 100%), calc. 359 for C₁₈H₁₉BrN₂O.

### Example 19

Conversion of Compounds of the Series (2), Example 18 into Boronate Derivatives

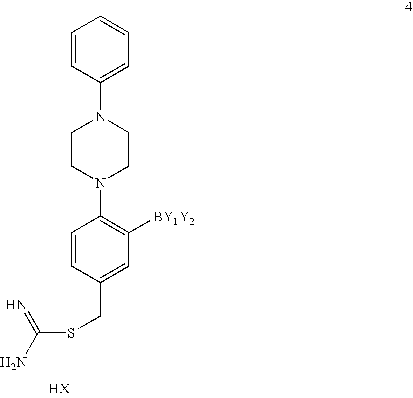

This (above) is made via the protected, ethylene glycol diacetal derivative of compound 2a (below):

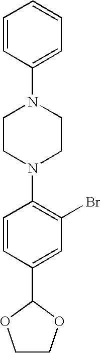

The diacetal derivative of 2a was prepared in >90% yield (estimated by NMR) by reaction of 2a and ethylene glycol according to the method described in Eur J. Org. Chem., 1998, 877-88. Selected ¹H and ¹³C NMR data (CDCl₃) δ 3.15 and 3.30 (2m, 8H), 4.01 and 4.06 (2m, 4H), 5.68 (s, 1H), 6.9-7.65 (m), 7.70 (s, 1H). ¹³C NMR (CDCl₃) δ 49.8, 52.0, 65.7, 103.1, 116-152.

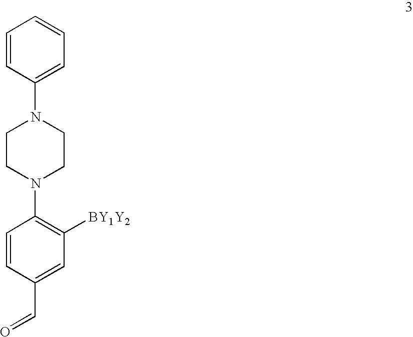

2a, ethylene glycol diacetal is metallated according to methods described in Synthesis Section Schemes 1(i) or (iii) to afford, after deprotection 3, from which 4 are made following the method developed for TRI 974.

### Example 20

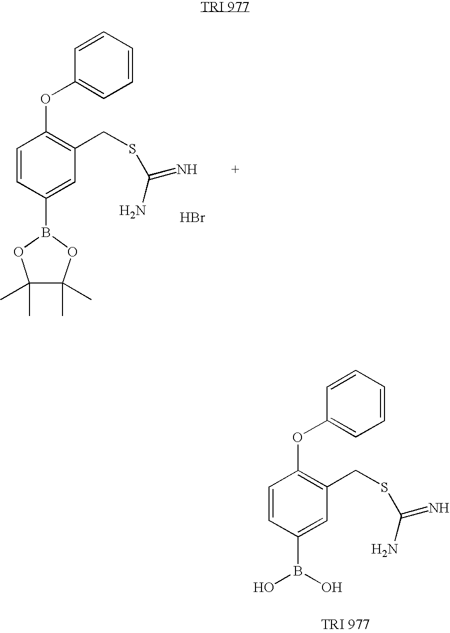

These compounds are made in a multistep procedure starting from 4-fluoro-3-formylbenzeneboronic acid, pinacol ester, involving the steps outlined below:

(a) 3-Formyl-4-phenoxybenzene boronic acid pinacol ester, an Intermediate.

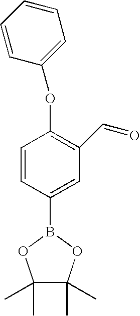

4-Fluoro-3-formylbenzeneboronic acid pinacol ester (1.25 g, 5 mmol), K₂CO₃ (700 mg, 5.1 mmol) and phenol (500 mg, 5.95 mmol) were heated overnight in dimethylacetamide (DMA) (50 ml) (following a general protocol by C. G. Yeager et al. Synthesis, 1994, 28-30). After cooling, the mixture was diluted with dichloromethane and washed with several portions of water. Following silica flash chromatography (Et₂O, neat), a yellow solid (850 mg, 52%) 3-Formyl-4-phenoxybenzene boronic acid pinacol ester was obtained. ¹H NMR (CDCl₃) δ 1.25 (s, 12H), 6.74 (d, 1H, J8), 7.00 (m, 2H), 7.12 (m, 1H), 7.30 (m, 2H), 7.82 (dd, 1H, J7, J2), 8.32 (d, 1H, J2), 10.46 (s, 1H). ¹³C NMR (CDCl₃) δ 23.8, 83.3, 116.0, 118.8, 122.2, 123.3, 127.3, 129.1, 134.7, 141.0, 154.6, 161.6, 188.4. MS (FAB) 325 (MH⁺, 25%) calc. 324 for C₁₉H₂₁BO₄.

(b) 3-hydroxymethyl-4-phenoxybenzene boronic acid pinacol ester, an Intermediate.

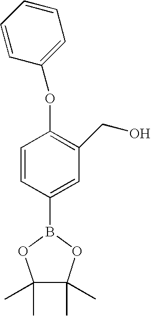

3-Formylphenoxybenzene boronic acid pinacol ester (350 mg, 1.08 mmol) and LiAlH₄ (70 mg, 1.9 mmol) were combined in anhydrous diethyl ether (10 ml) then heated to reflux overnight. After cooling, a further batch of LiAlH₄ (70 mg, 1.9 mmol) was added and the mixture was stirred overnight. The reaction mixture was carefully quenched by the addition of water and HCl (1N, 5 ml) and diluted with further diethyl ether. The organic layer was separated and dried (MgSO₄) before concentration. The product, 3-hydroxymethyl-4-phenoxybenzene boronic acid pinacol ester, was obtained after flash chromatography over a silica gel column (257 mg, 73% with neat Et₂O as eluant). ¹H NMR (CDCl₃) δ 1.25 (s, 12H), 4.69 (S, 2H), 6.72 (d, 1H, J7), 6.90 (m, 2H), 6.93 (m, 1H), 7.25 (m, 2H), 7.70 (d, 1H), 7.82 (d, 1H, J2). ¹³C NMR (CDCl₃) δ 23.8, 60.1, 82.8, 114.3, 118.0, 122.2, 127.9, 128.2, 129.7, 134.9, 156.0, 156.8.

The title compound was made as follows:

3-bromomethyl-4-phenoxybenzene boronic acid pinacol ester was formed in situ by reacting 3-hydroxymethyl-4-phenoxybenzene boronic acid pinacol ester (190 mg, 0.54 mmol) with a 30% solution of HBr/CH₃CO₂H (0.5 ml) in ether (5 ml) overnight and worked-up according to the method employed in Example 22 (see below). The crude product (180 mg) was stirred in methanol (5 ml) for 72 h with thiourea (35 mg, 0.46 mmol) at rt. The reaction mixture was concentrated to afford crude material (58 mg), comprising mainly the pinacol ester derivative.

¹H NMR (DMSO d-₆) δ 1.28 (s, 12H), 4.55 (s, 2H), 6.7-8.1 (aromatics), 9.1 & 9.3 (2brs, 4H). MS (EI, MeOH) 385 (MH⁺, 100) calc. 384 for C₂₀H₂₅BN₂O₃S.HBr. (Rt=24.2 min, gradient 1-60% MeCN/water, RP on Phenomenex Luna Analytical Column). Trituration of the above mixture afforded a white precipitate TRI 977 (2 mg from CH₂Cl₂/Et₂O at −5° C. overnight). ¹H NMR (DMSO d-₆) δ 4.50 (s, 2H), 6.75 (d, 1H, J 8), 7.04 (d, 1H, J 7), 7.22 (t, 1H, J 7), 7.44 (t, 2H), 7.74 (d, 1H, J 9), 7.89 (s, 1H), 8.12 (s, 2H), 9.16 (2brs, 4H). MS (EI, MeOH) 331 (MH+ bis-MeOH adduct, 100%), 317 (MH+ mono-MeOH adduct, 50%) calc. 302 for C₂₀H₂₅BN₂O₃S.HBr. (Rt=11.2 min, gradient 1-60% MeCN/water, RP on Phenomenex Luna Analytical Column).

### Example 21

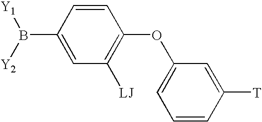

3-Formyl-4-(3-nitrophenoxy)benzene boronic acid pinacol ester, an Intermediate

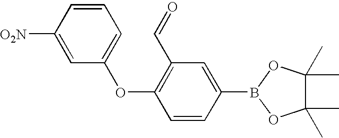

This compound was prepared, as in Example 20, from 4-Fluoro-3-formylbenzeneboronic acid pinacol ester (1 g, 4 mmol), K₂CO₃ (850 mg, excess) and 3-nitrophenol (600 mg, 4.31 mmol) in DMA (20 ml) as a white solid which was obtained in several small crops by repeated trituration in acetone (590 mg, 40%). ¹H NMR (CDCl₃) δ 1.29 (s, 12H), 6.87 (d, 1H, J 8), 7.35 (t, 1H, J 2), 7.50 (t, 1H, J 8), 7.81 (d, 1H, J 2), 7.92-7.97 (m, 2H), 8.37 (d, 1H, J 2), 10.36 (s, 1H). ¹³C (CDCl₃) δ 25.2, 84.7, 114.3, 118.7, 119.4, 125.5, 127.1, 131.2, 137.3, 142.7, 149.8, 157.5, 160.4, 189.0. Anal. Calc. (found), C, 61.81 (61.66), H, 5.46 (5.39), N, 3.79 (3.90).

### Example 22

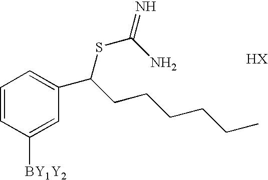

a) 3-formylbenzeneboronic acid, pinacol ester 1 and neopentyl glycol ester 2, Intermediates.

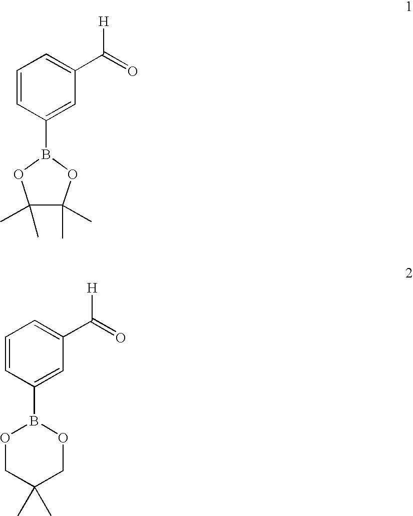

3-Formylbenzeneboronic acid (3.5 g, 23 mmol) and pinacol (2.7 g, 23 mmol) were stirred in anhydrous THF (30 ml) for 3 h with 3A molecular sieves. After filtration, the filtrate was concentrated in vacuo affording an orange oil 3-formylbenzeneboronic acid, pinacol ester 1 (5.1 g, 96%). ¹H NMR (CDCl₃) δ 1.29 (s, 12H), 7.46 (t, 1H, J7), 7.92 (d, 1H), 7.99 (d, 1H), 8.23 (s, 1H), 9.98 (s, 1H). ¹³C NMR (CDCl₃) δ 25.3, 84.7, 128.8, 131.7, 136.1, 137.7, 141.1, 193.0.

The neopentylglycol ester derivative 2 was made in an analogous fashion to its pinacol derivative, except that neopentyl glycol was employed as diol to give 3-formylbenzeneboronic acid, neopentyl glycol ester ¹H NMR (CDCl₃) δ 0.97 (s, 6H), 3.73 (s, 4H), 7.45 (t, 1H, J8), 7.88 (d, 1H), 7.99 (d, 1H), 8.23 (s, 1H), 9.97 (s, 1H). ¹³C NMR (CDCl₃) δ 22.3, 68.4, 72.8, 128.8, 131.2, 136.1, 136.8, 140.3, 193.3.

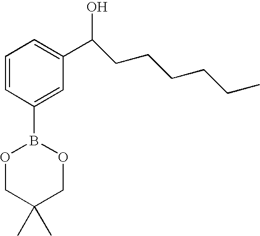

b) 1-(3-phenylboronic acid)hexylmethanol neopentylglycol ester, an Intermediate.

Hexylmagnesium bromide (0.6 ml, 1.22 mmol, 2M solution in THF) was added dropwise to a cooled THF (10 ml) solution of 3-formylbenzeneboronic acid neopentyl glycol ester (as above; 217 mg, 1 mmol) at −78° C. After allowing the reaction mixture to warm to 0° C., the solvent was removed in vacuo. A solution of HCl (0.1 N, 10 ml) and ethyl acetate (10 ml) were added and the organic layer was separated and washed with brine. After MgSO₄ drying, filtration and concentration in vacuo, an oil (3-phenylboronic acid)heptanol neopentylglycol ester (170 mg, 56%) was obtained. ¹H NMR (CDCl₃) δ 0.77-1.7 (m, 12H), 0.95 (s, 6H), 2.54 (s, 2H), 3.70 (s, 4H), 7.24-7.95 (m, 4H), ¹³C DEPT 135 NMR (CDCl₃) δ 14.5, 22.3, 30.0, 127.3-139.5 (all +ve), 23.0, 24.3, 30.0, 32.1, 44.6, 72.6 (all −ve). MS (ESI, MeOH) 301 (M⁺−3H, 100%) calc. 304 for C₁₈H₂₉BO₃.

(3-phenylboronic acid)heptanol neopentylglycol ester is converted to the title (3-phenylboronic add)heptyl-1-thiouronium neopentylglycol ester product by the methods of Scheme 3(ii) and 4(i) under the heading “Synthesis”.

### Example 23

Route to m-(1-isothiouronium)ethylbenzene boronic acid pinacol ester TRI 974 from 3-acetylbenzene boronic acid.

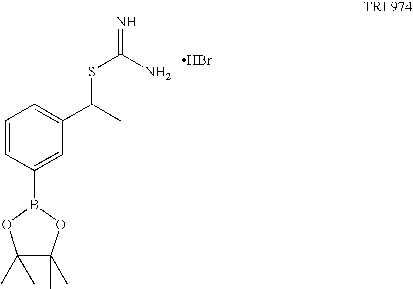

a) 3-acetylbenzeneboronic acid, pinacol ester 1 and neopentyl glycol ester 2, Intermediates.

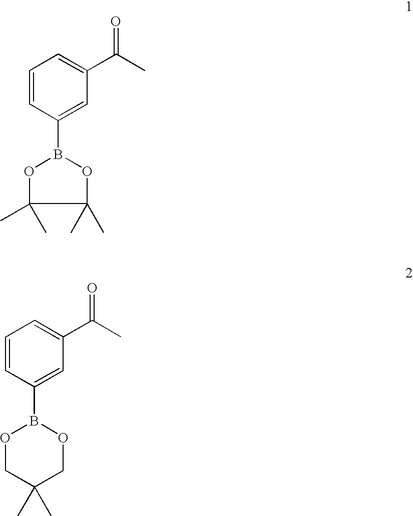

The 3-acetylbenzeneboronic acid pinacol derivative was synthesised as for its 3-formylbenzeneboronic acid analogue (c.f. Example 21) except starting from 3-acetylbenzene boronic acid and pinacol. ¹H NMR (CDCl₃) δ 1.28 (s, 12H), 2.55 (s, 3H), 7.39 (t, 1H, J7), 7.91 (d, 1H), 7.98 (d, 1H), 8.28 (s, 1H). ¹³C NMR (CDCl₃) δ 25.3, 27.2, 84.6, 128.5, 131.2, 135.2, 136.9, 139.8, 198.8. The neopentyl glycol analogue was made in an analogous fashion to its pinacol derivative, except that neopentyl glycol was employed as diol. ¹H NMR (CDCl₃) δ 0.97 (s, 6H), 2.57 (s, 4H), 3.72 (s, 4H), 7.38 (t, 1H, J7), 7.92 (d, 1H), 7.97 (d, 1H), 8.30 (s, 1H). ¹³C NMR (CDCl₃) δ 22.3, 32.3, 68.4, 72.7, 128.3, 130.6, 134.4, 136.8, 138.9, 199.1.

b) m-(1-ethoxybenzene)boronic acid pinacol ester, an Intermediate.

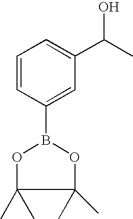

An ethereal solution (5 ml) of 3-acetylbenzeneboronic acid, pinacol ester (250 mg, 1 mmol) was added to an ether suspension (10 ml) of LiAlH₄ (37 mg, 1 mmol). The reaction mixture was stirred overnight and then quenched by the addition of ethyl acetate (2 ml). After filtration over Celite, to remove the grey slurry, the filtrate was concentrated in vacua. The grey slurry was dissolved in 1N HCl (20 ml) and extracted with ethyl acetate (10 ml). The organic layer was washed with brine and dried (MgSO₄) then combined with the earlier filtrate. Concentration afforded an off-white oil 150 mg (60%). ¹H NMR (CDCl₃) δ 1.27 (s, 12H), 1.43 (d, 3H, J6), 4.84 (q, 1H), 7.29 (t, 1H, J7), 7.43 (d, 1H), 7.65 (d, 1H, J7), 7.71 (s, 1H). ¹³C NMR (CDCl₃) δ 19.7, 23.8, 69.4, 82.9, 126.9, 127.3, 130.7, 132.9, 143.9.

c) m-(1-methanesulphonylethoxybenzene)boronic acid pinacol ester, an Intermediate.

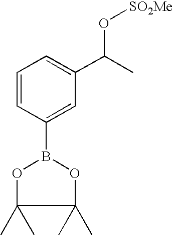

m-(1-hydroxy)ethylbenzeneboronic acid pinacol ester (150 mg, 0.6 mmol) was dissolved in dry dichloromethane (10 ml) and triethylamine (200 μl, 1.5 mmol) was added followed by methanesulphonyl chloride (70 μl). The mixture was stirred overnight then diluted with further dichloromethane (10 ml) and washed with 1N Ha (10 ml) and brine (10 ml) then dried over MgSO₄. Concentration afforded a red oil (210 mg). ¹H NMR (CDCl₃) δ 1.28 (s, 12H), 1.78 (d, 3H, J7), 3.60 (s, 3H), 5.04 (q, 1H), 7.30 (t, 1H, J7), 7.48 (m, 1H), 7.67 (d, 1H, J7), 7.76 (s, 1H). ¹³C (CDCl₃) δ 25.3, 26.9, 52.9, 59.3, 84.3, 128.5, 129.8, 133.1, 135.1, 142.5.

d) m-(1-bromoethylbenzene)boronic acid pinacol ester

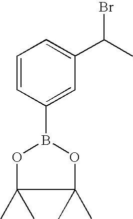

m-(1-hydroxy)ethylbenzene boronic acid pinacol ester (160 mg, 0.63 mmol) was dissolved in anhydrous ether (5 ml) and a 30% solution of HBr/CH₃CO₂H (0.7 ml, excess, see Eur. J. Org. Chem., 1998, 877) was added. The mixture was stirred at room temperature for a further 1.5 h then diluted with ether (10 ml) and carefully washed with a saturated aqueous solution of sodium bicarbonate. After MgSO₄ drying, filtration, the filtrate was concentrated in vacua to afford a pale yellow oil, 180 mg (92%). ¹H NMR (CDCl₃) δ 1.28 and 1.49 (2×s, 2×12H, free pinacol present), 1.99 (d, 3H, J8), 5.16 (q, 1H), 7.29 (t, 1H, J7), 7.50 (m, 1H), 7.66 (d, 1H, J7), 7.76 (s, 1H). ¹³C NMR (CDCl₃) δ 25.3, 27.2, 50.1, 59.3, 84.3, 128.6, 130.2, 133.2, 135.2.

Alternatively, this intermediate can be made using m-(1-hydroxy)ethylbenzeneboronic acid pinacol ester and PPh₃Br₂ as brominating agent. In this case, conversion to the bromide intermediate was observed by NMR, although purificaton by silica gel chromatography resulted in its decomposition (loss of HBr to give styryl product).

e) TRI 974

m-(1-bromo)ethylbenzeneboronic acid pinacol ester (900 mg, 2.9 mmol) and thiourea (228 mg, 3 mmol) were stirred in methanol (10 ml) at rt for 60 h. The mixture was concentrated in vacua. Initial trituration in dichloromethane followed by overnight trituration in ether afforded an off-white solid, which was dried in vacua (120 mg, 10%). ¹H NMR (DMSO-d₆) δ 1.30 (s, 12H), 1.63 (d, 3H, J7), 5.14 (q, 1H), 7.43 (m, 1H), 7.59 (m, 2H), 7.75 (s, 1H), 8.99 (brs, 2H), 9.22 (brs, 2H). ¹³C (DMSO-d₆) a 22.2, 25.0, 44.6, 84.2, 128.9, 130.5, 133.9, 134.6, 140.3, 168.2. MS (ESI, MeOH) 339 (M++MeOH, 30%), 307 (M H⁺, 100%) calc. 306 for C₁₅H₂₃BN₂O₂S.HBr.

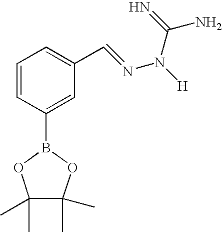

### Example 24

This compound is prepared from m-formylbenzene boronate ester (example 20) by the method of R. M. Soll et al, Bio-Org. Med. Chem. Lett, 2000, 10,1-4.

### Example 25

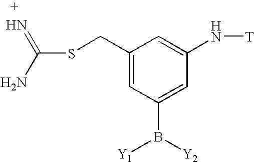

The above compound is made from the intermediate 1 by a series of transformations as outlined in section 10 and 11 in the “Synthesis” section.

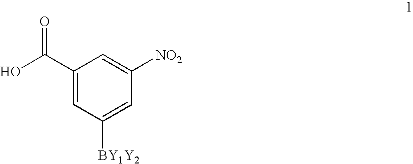

### Example 26

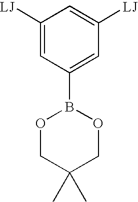

a) 3,5-dimethylcarboxybenzeneboronic acid neopentylglycol ester, an Alternative Intermediate to Compounds of the type of TRI 900 and 968.

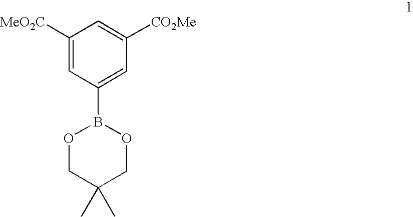

Dimethyl-5-bromoisophthalate (2.73 g, 10 mmol) was combined with bis(neopentyl) glycolatodiboron (2.75 g, 12.2. mmol), PdCl₂dppf (250 mg, 2.5 mol %) and sodium acetate (1.3 g, 15.8 mmol) in MeOH (30 ml). The mixture was purged with argon then heated to reflux overnight. After cooling, the mixture was diluted with ether, filtered over a plug of Celite and concentrated. The mixture was purified by flash chromatography over silica (dichloromethane as eluant) to afford 240 mg of product (8%). ¹H NMR (CDCl₃) δ 1.42 (s, 6H), 3.80 (s, 4H), 3.94 & 4.00 (2s, 6H), 8.52, 8.63, 8.73 (3s, 3H). ¹³C (CDCl₃) δ 21.9, 26.9, 52.4, 72.4, 128.6, 129.9, 130.7, 132.8, 133.8, 135.1, 166.1. MS (FAB) 307 (MH⁺, 100) calc 306 for C₁₅H₁₉BO₆.

1 is converted to the title compound by a series of transformations, involving reduction or alkylation of the carbonyl groups, as outlined in Examples 22 and 23.

### Example 27

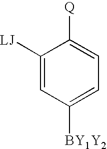

a) 4-Bromo-2-methylbenzoic acid ethyl ester (1), an intermediate.

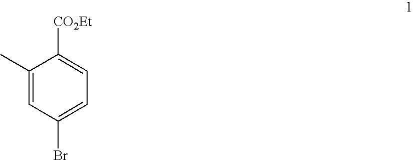

4-Bromo-2-methylbenzoic acid (2.2 g, 10.3 mmol) was treated with excess thionyl chloride in toluene (50 ml). The mixture was refluxed and a catalytic amount of DMF was added, after which, the white suspension dissolved. After 3 h reflux, the reaction mixture was cooled and concentrated then poured into ethanol (20 ml). The mixture was left to stir overnight. After concentration, the residue was extracted with ether, carefully washed with saturated sodium bicarbonate and dried over MgSO₄. Filtration, then concentration afforded an orange liquid, 1.98 g, which was used without further purification. ¹H NMR (CDCl₃) δ 1.38 (t, 3H, J 7), 2.57 (s, 3H), 4.34 (q, 2H), 7.38 (m, 2H), 7.78 (d, 1H, J 8). ¹³C DEPT 135 (CDCl₃) δ 14.3, 21.6, 128.9, 132.0, 134.5 (+ve), 60.9 (−ve).

b) 4-carboxyethyl-3-methyl-phenylboronic acid neopentyl glycol ester, an Intermediate.

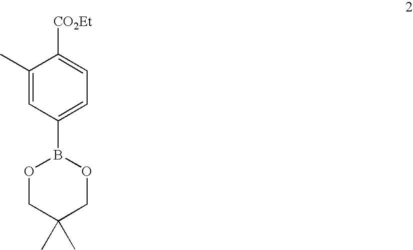

This was synthesized in 71% yield (195 mg) starting from 4-bromo-2-methylbenzoic acid ethyl ester 1 (240 mg, 1 mmol), bis(neopentyl)glycolato)diboron (275 mg, 1.2 mmol), PddppfCl₂ (25 mg, 2.5%) and sodium acetate (130 mg; 1.6 mmol) in MeOH (10 ml) using the same procedure as in Example 26. ¹H NMR (CDCl₃) δ 0.90 & 1.02 (2s, 6H), 1.39 (t, 3H, J7), 2.59 (s, 3H), 3.49 & 3.78 (2s, 4H), 4.36 (q, 2H), 7.66 (m, 2H), 7.86 (d, 1H, J7). ¹³C NMR (CDCl₃) δ 14.3, 21.3, 21.6, 28.9, 36.5, 60.7, 71.6, 72.4, 124.5, 129.4, 131.7, 138.6, 168.0. FAB 277 (MH⁺, 90%), 231 (100%, M-OEt) calc 276 C₁₅H₂₁BO₄.

2 is converted into the title compound following the synthetic procedures as outlined in Examples 2 and 4(i) (transformation into LJ group) and 3 (manipulation of Q).

### Example 28

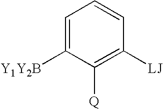

a) 2-iodo-6-methylbenzoic acid methyl ester, an Intermediate.

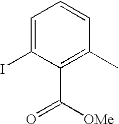

This was made from 2-iodo-6-methylbenzoic acid, thionyl chloride and methanol in an analogous fashion to the method described in Example 24. ¹H NMR (CDCl₃) δ 2.33 (s, 3H), 3.95 (s, 3H), 6.98 (t, 1H, 38), 7.17 (d, 1H), 7.64 (d, 1H, 38). ¹³C DEPT 135 NMR (CDCl₃) δ 20.0, 52.6, 129.7, 130.7, 136.3 (all +ve).

2-Iodo-6-methylbenzoic acid methyl ester is converted into the title compound using the methods described in Example 26 (boronation step) followed by steps as outlined in Examples 2 and 4(i) (transformation into U group) and 3 (manipulation of Q).

### Example 29

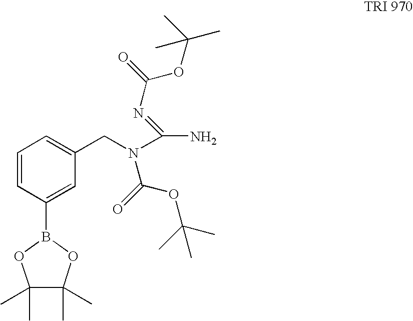

N¹,N²-bis(t-butyloxycarbonyl)guanidine (259 mg, 1 mmol) was added dropwise to a DMF suspension (5 ml) of sodium hydride (42 mg, 1.1 mmol of an unwashed 60% oil suspension). After stirring for 10 min, 3-bromomethylphenylboronic acid pinacol ester (297 mg, 1 mmol), in DMF (5 ml), was added and the mixture was stirred for 2 h at rt. The reaction mixture was poured into water and extracted with ethyl acetate and the organic phase was washed several times with water and brine before being dried over MgSO₄. Filtration and concentration yielded a pale oil which solidified over time to afford a white solid, 290 mg (61%). ¹H NMR (CDCl₃) a 1.34 (s, 12H), 1.35 & 1.49 (2s, 18H), 5.19 (s, 2H), 7.30 (m, 1H), 7.40 (m, 1H), 7.68 (d, 1H, J7), 7.72 (s, 1H), 9.43 (brs, 2H). ¹³C (CDCl₃) δ 24.9, 47.4, 84.1, 127.7, 128.8, 130.1, 132.0, 137.9, 155.0, 160.9, 162.5, 163.8. MS (ESI) 478 (M3H+, 20%), 276 (MH-2Boc, 100%) calc. 475 for C₂₄H₃₈BN₃O₆. Anal. Calc. (found) C, 60.64 (60.81), H, 8.12 (8.06), N, 8.89 (8.84). An alternative route to TRI 970 involved 3-hydroxymethylphenylboronic acid pinacol ester (1 g, 4 mmol, made from TRI 838 and pinacol as in Example 18a), N¹,N²-bis(t-butyloxycarbonyl)guanidine (1.5 g, 6 mmol), triphenylphosphine (1.57 g, 6 mmol) and 1,1′(azodicarbonyl)dipiperidine (1.5 g, 6 mmol) in THF (10 ml) as for D. S. Dodd, A. Kozikowski, Tetrahedron Lett. 1994, 35, 977. TRI 970 (850 mg, 45%) was obtained after chromatography (SiO₂, Ethyl acetate:hexane, 1:4, R_{f}=0.5, spectral data identical to above).

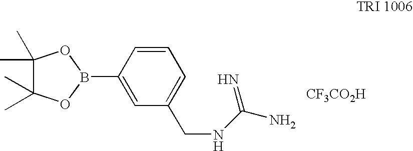

TRI 970 (238 mg, 0.5 mmol) was stirred with a 1.1 mixture of TFA/dichloromethane (4 ml) for 5 h at rt followed by concentration in vacuo to afford the crude colourless oil. ¹H NMR (DMSO-d₆) δ 1.21 (s, 12H), 4.29 (d, 2H, J 5), 7.33 (m, 3H), 7.56 (m, 3H), 7.80 (m, 1H). ¹³C NMR (DMSO-d₆) δ 25.0, 44.1, 84.1, 128.5, 130.8, 133.6, 133.9, 137.1, 157.1. MS (ESI, MeOH) 551 (2M+, 20), 276 (M+H, 100).

### Example 30

Direct Synthesis of Isothiouronium Product from a Benzyl Alcohol and Thiourea.

a) 3-hydroxymethyl-4-(4-benzylpiperazino)phenylboronic acid pinacol ester, an Intermediate

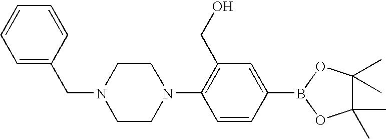

Under Argon, 3-formyl-4-(4-benzylpiperazino)phenylboronic acid pinacol ester (Example 18, 1f, 250 mg, 0.62 mmol) and LiAlH₄ (85 mg, 2.3 mmol, excess) were combined in anhydrous tetrahydrofuran (10 ml) then refluxed overnight. After cooling, ethyl acetate (10 ml) was added to the reaction mixture as well as silica (ca. 5 g) before concentration in vacuo. The thereby-preloaded product was loaded onto a silica column and eluted with neat Et₂O(R_{f}=0.7) to afford a yellow oil (150 mg, 59%). ¹H NMR (CDCl₃) δ 1.25 (s, 12H), 2.56 (m, 4H), 2.95 (s, 4H), 3.50 (s, 2H), 4.72 (s, 2H), 7.09 (m, 1H), 7.12-7.25 (m, 5H), 7.56 (d, 1H, J 2), 7.64 (dd, 1H, J8, J2). ¹³C (CDCl₃) δ 25.2, 52.5, 54.0, 63.4, 65.1, 84.1, 120-135, 158. MS (ESI, MeOH) 409 (M+H, 100) calc 408 for C₂₄H₃₉BN₂O₃.

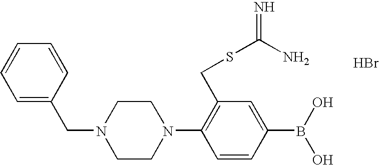

3-hydroxymethyl-4-(4-benzylpiperazino)phenylboronic acid pinacol ester (316 mg, 0.77 mmol) and thiourea (60 mg, 0.78 mmol) were heated to reflux in aqueous hydrobromic acid (4.5 ml, 48% solution) for 3 hours. After cooling, the reaction mixture was concentrated in vacuo and the residue extracted with water (25 ml). After filtration, the filtrate was concentrated in vacuo then washed with ether then dichloromethane to afford an orange oil (60 mg, 17%). ¹H NMR (DMSO-d₆) δ 3.17 (m, 4H), 3.41 (m, 4H), 4.50 (s, 4H), 7.19-7.48 (m, 6H), 7.68 (brs, 2H), 9.08 (m, 4H). ¹³C (DMSO-d₆) δ 30.9, 49.5, 51.4, 55.3, 121.9, 126.0, 129.3, 129.9, 130.0, 131.0, 131.4, 131.9, 150.4, 170.0. MS (ESI, MeOH) 400 (M+OH, 10%), 341 (M-C(═NH)NH₂,100) calc 384 for C₁₉H₂₅BN₄O₂S.2HBr.

### Example 31

Direct Synthesis of Isothiouronium Product from a Benzyl Alcohol and Thiourea.

a)₃-hydroxymethyl-4-(2-pyridylpiperazino)phenylboronic acid pinacol ester, an Intermediate.

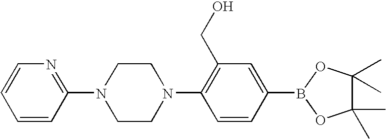

3-formyl-4-(2-pyridylpiperazino)phenylboronic acid pinacol ester (Example 18, 1b, 500 mg, 1.28 mmol) was added dropwise to a suspension of LiAlH₄ (110 mg, 2.97 mmol) in THF (10 ml) at rt then the reaction mixture was heated to 40° C. overnight. After cooling, the reaction was quenched by the addition of ethyl acetate (5 ml) and silica and the resulting suspension was concentrated in vacuo before being loaded on a flash silica column with ethyl acetate as eluant. The product was obtained as a yellow solid (200 mg, 40%). ¹H (CDCl₃) 1.21 (s, 12H), 3.05 (m, 4H), 3.64 (m, 4H), 4.78 (s, 2H), 6.59 (m, 2H), 7.12 (m, 1H), 7.45 (t, 1H, 37), 7.62 (s, 1H), 7.67 (m, 1H), 8.14 (dd, 1H, J5, J2). ¹³C (CDCl₃) 25.2, 46.2, 52.5, 64.7, 84.2, 107.6, 114.2, 120.0, 134.8, 135.7, 135.9, 138.0, 148.4, 153.8. MS (ESI, MeOH) 396 (MH⁺, 100) calc 395 for C₂₂H₃₀BN₃O₃.

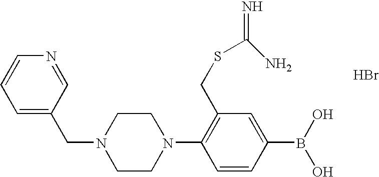

3-hydroxymethyl-4-(2-pyridylpiperazino)phenylboronic acid pinacol ester (160 mg, 0.40 mmol) and thiourea (31 mg, 0.40 mmol) were heated to reflux in aqueous hydrobromic acid (3 ml, 48% solution) for 2 hours. After cooling, the reaction mixture was concentrated in vacuo and the residue was triturated overnight in acetone (10 ml) to afford a white solid (120 mg, 66%). ¹H NMR (DMSO-d₆) δ 3.09 (m, 4H), 3.96 (m, 4H), 4.59 (s, 2H), 7.00 (m, 1H), 7.02-7.55 (m, 4H), 8.06 (m, 2H), 9.19 (m, 3H). MS (ESI, MeOH) 400 (M+ bis MeOH adduct, 10), 386 (mono MeOH adduct, 20), 328 (M-C(═NH)NH₂, 100). calc 371 for C₁₇H₂₂BN₅O₂S.

Tables

The invention is further illustrated by reference to the following tables, in which:

Table 1 shows the effectiveness measured by “score” (see below) of comparative compounds not failing within the scope of the invention;

Table 2 lists certain intermediate compounds useful for making compounds of the invention; and

Table 3 shows the effectiveness measured by “score” of compounds of the invention.

Score

Objective:

Score gives a facile measure of the effectiveness of an inhibitor requiring only one or two data points, rather than the twelve or more data points required to calculate an IC₅₀.

The principle behind calculating the score is that, although the absolute values obtained from inhibitors of the same type may be different, the shape and range of the IC₅₀ curves are quite similar.

Apparatus:

Molecular Devices Corp. Thermomax plate reader.

Reagents:

chromogenic substrates

S-2238 (H-D-Phe-Pip-Arg-pNA, for Thr) was obtained from Quadratech.

S-2288 (H-D-Ileu-Pro-Arg-pNa for Try) was obtained from Quadratech.

S-2251 (H-D-Val-Leu-Lys-pNa for Pla) was obtained from Quadratech.

S-2765 (N-α-Z-D-ArgGly-Arg-pNa for Xa) was obtained from Quadratech.

S-2444 (pyro-Glu-Gly-Arg-pNa for UK) was obtained from Quadratech.

Pefachrome IXa (Kordia, for FIXa)

Reagents:

Buffer for Thr, Try, Pla, UK or Xa assay: 0.1M sodium phosphate; 0.2M sodium chloride; 0.5% PEG6000; 0.02% sodium azide, pH 7.4.

Buffer for FIXa assay: 50 mM Tris; 100 mM sodium chloride; 0.5% PEG 6000, pH 7.4. The aqueous buffer was freshly mixed each day with ethylene glycol (65%:35% respectively) Human α-thrombin was obtained from J. Freysinnet (Strasbourg).

FIXa was obtained from Haematologics.

Typical Volumes:

Thr Assay:

Inhibitor (100 μl of dilution in buffer of 1/20 or higher as appropriate from stock in DMSO) is added to substrate (50 μl of a dilution of 1/100 of 2.4 mg/ml stock) for 2 minutes at 37° C. Thr (50 μl of a dilution of 1/10,000 of stock at 1 mg/ml) is added.

Final Substrates Concentrations were 25 μM, 113 μM, 22 μM and 125 μM for Try, Pla, Xa and UK Respectively.

FIXa Assay:

Inhibitor (100 μl of a dilution in buffer of 1/20 or higher from stock in DMSO) is added to substrate (50 μl of a 1.67 mM solution in buffer) for 2 minutes at 37° C. FIXa (50 μl of a stock at 0.01 mg/ml) is added.

Standard Method of Determination of IC₅₀ for Test Compounds:

A solution of each enzyme was added to a series of 10 fold dilutions of each inhibitor in the presence of a fixed concentration of the appropriate chromogenic substrate in order to determine the inhibitor concentration needed to give approximately 50% inhibition. A series of concentrations of inhibitor either side of this approximate 50% value (at least 5 different inhibitor concentrations each point measured in duplicate) were used to construct a graph of % inhibition against inhibitor concentration. From this graph the exact inhibitor concentration needed to give 50% inhibition can be measured (IC₅₀).

Calculation

Determination of IC₅₀ After Preincubation:

For inhibitors showing possible slowbinding characteristics the enzyme and each dilution of inhibitor were incubated at 37° C. in the absence of substrate for 10 minutes. The reaction was started with the addition of substrate and the IC₅₀ determined as above.

Method for Calculating Score:

Average IC₅₀ Curve:

The data from IC₅₀ curves for 13 compounds were used to construct an average IC₅₀ curve. For each individual IC₅₀ curve the concentration of inhibitor needed to give 5% inhibition was arbitrarily set at a value of 1.

The fold increase in inhibitor concentration needed to increase the % inhibition in steps of 10% was measured from each respective IC₅₀ curve. The average values for the fold increase at each 10% step are shown below:

TABLE A

| Plate reader | | fold increase in inhibitor |
|---|---|---|
| observed value | % inhibition (range) | concentration |
| 9 | 5 (0 to 10) | 1 |
| 8 | 15 (11 to 20) | 2.4 |
| 7 | 25 (21 to 30) | 4.2 |
| 6 | 35 (31 to 40) | 6.4 |
| 5 | 45 (41 to 50) | 8.8 |
| 4 | 55 (51 to 60) | 12.1 |
| 3 | 65 (61 to 70) | 16.7 |
| 2 | 75 (71 to 80) | 24.4 |
| 1 | 85 (81 to 90) | 41.3 |
| 0 | 95 (91 to 100) | 99.2 |

Calculation:

Measurement of single concentrations of inhibitor on a plate reader (using MDC ‘Softmax’ software) gave a value from 9 to 0 when compared to a control without inhibitor which corresponds to the % inhibition a shown in Table 1. Provided that the plate reader number was >2 but <8 (linear part of the graph) the score could then be calculated as follows.

Score=fold increase in inhibitor concentration (see table 1)/concentration of inhibitor used (in mM)

### EXAMPLE

e.g. if a 0.1 mM inhibitor gave an observed value on the plate reader of 5 (between 41% and 50% inhibition)

then: Score=8.8/0.1=88

TABLE 1
Comparators: non- H-bond acceptor isothiouronium and guanidine compounds

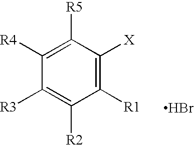

| | | | | | | | FIXa | Thr |
|---|---|---|---|---|---|---|---|---|
| TRI No. | X | R1 | R2 | R3 | R4 | R5 | score | Score |
| 885 | I | H | CH₂S—C(═NH)NH₂ | H | H | H | 0.6 | 10 |
| 887 | CO₂CH₃ | H | CH₂S—C(═NH)NH₂— | H | H | H | 0.9 | 5.3 |
| 929* | Br | F | H | F | H | NH(C═NH)NH(C═NH)NH₂ | 1.6 | 0 |
| 930* | I | H | NH(C═NH)NH(C═NH)NH₂ | CH₃ | H | H | 0.6 | 0 |
| 931* | Br | H | i-Pr | H | H | NH(C═NH)NH(C═NH)NH₂ | 0.8 | 7.7 |
| 932* | I | H | H | NHC(═NH)NHC(═)NH₂ | H | H | 0.3 | 0 |

Legend table 1:
*Indicates commercially available.

TABLE 2
Intermediates

| | | | | | | X = B(OH)₂ |
|---|---|---|---|---|---|---|
| | | | | | | unless |
| | | | | | | otherwise |
| TRI No. | R1 | R2 | R3 | R4 | R5 | specified |
| 743* | CHO | H | H | OMe | H | |
| 744* | CHO | H | OMe | H | H | |
| 745* | H | H | CH₂CH₂CO₂H | H | H | |
| 746* | H | H | CO₂H | H | H | |
| 747* | H | H | CHO | H | H | |
| 755 | H | H | CO₂Succ | H | H | pinanediol |
| | | | | | | ester |
| 756 | H | H | CH₂CH₂CO₂H | H | H | pinacol ester |
| 758* | CHO | H | H | H | H | |
| 765* | CH₂Br | H | H | H | H | |
| 766* | NH₂ | H | H | H | H | pinacol ester |
| 768* | H | NH₂ | CH₃ | H | H | |
| 770* | H | H | NH₂ | H | H | |
| 774* | CO₂H | H | H | H | H | |
| 775* | H | NHCOCH₃ | H | H | H | |
| 776* | H | NH₂ | H | H | H | |
| 838 | H | CH₂OH | H | H | H | |
| 855* | H | H | CN | H | H | |
| 856* | H | CN | H | H | H | |
| 863* | H | H | SO₂CH₃ | H | H | |
| 867* | CH₂Br | H | H | H | H | |
| 868* | H | CH₂Br | H | H | H | |
| 869* | H | H | CH₂Br | H | H | |
| 917* | H | Br | H | Br | H | |
| 918* | H | CH₃ | H | CH₃ | H | |
| 919* | H | F | H | F | H | |
| 920* | CH₂NMe₂ | H | H | H | H | |
| 921* | H | OMe | OMe | H | H | |
| 922* | H | CF₃ | H | CF₃ | H | |
| 923* | F | F | Br | H | H | |
| 924* | OMe | H | H | CHO | H | |
| 925* | H | CHO | F | H | H | |
| 928* | H | Cl | F | H | H | |
| 933* | H | H | CH₃ | NHCONH₂ | H | |
| 934* | H | H | CO₂H | NO₂— | H | |
| 966 | H | NHCONH₂ | H | CO₂H | H | |
| 969 | H | CHO | N(CH₂CH₂)₂NPh | H | H | Pinacol ester |
| 971 | H | CHO | N(CH₂CH₂)₂NMe | H | H | Pinacol ester |

Legend Table 2:
*indicates commercially available

TABLE 3
Final product aryl boronate and nitro aryl protease inhibitors.

| | | | | | | X = B(OH)₂ | | | | | | |
|---|---|---|---|---|---|---|---|---|---|---|---|---|
| | | | | | | unless | | | | | | |
| TRI | | | | | | otherwise | FIXa | Thr | | | | |
| No. | R1 | R2 | R3 | R4 | R5 | specified | score | Score | Try | Xa | Pla | UK |
| 832 | CH₂S— | H | H | H | H | pinacol | 7 | 70 | 7 | 7 | 1 | 26 |
| | C(═NH)NH₂ | | | | | ester | | | | | | |
| 852 | H | CH₂S—C(═NH)NH₂ | H | H | H | pinacol | 76 | 326 | 624 | 624 | 393 | 792 |
| | | | | | | ester | | | | | | |
| 864 | H | CH₂S—C(═NH)NH₂ | H | H | H | | 209 | 421 | 868 | 868 | 289 | 776 |
| 865 | CH₂S— | H | H | H | H | | 5.4 | 70 | 7 | 7 | 3 | 26 |
| | C(═NH)NH₂ | | | | | | | | | | | |
| 866 | H | H | CH₂S—C(═ | H | H | | 6.4 | 6.4 | 440 | 6 | 32 | 3076 |
| | | | NH)NH₂ | | | | | | | | | |
| 896 | H | CH₂S—C(═NH)NH₂ | H | H | H | NO₂ | 2 | 295 | 4 | 4 | 2 | 3 |
| 903 | H | CH₂S—C(═NMe)NH₂ | H | H | H | | 19 | 38.7 | 21 | 39 | 21 | 27 |
| 915 | H | CH₂S— | H | H | H | | 10.1 | 2.0 | 2 | 1 | 1 | 4 |
| | | C(═NH)NHNHCOCH₃ | | | | | | | | | | |
| 967 | H | CH₂SC(═NH)NH₂ | H | Me | H | | 205 | 1645 | 3 | 905 | 312 | 785 |
| 968 | H | CH₂SC(═NH)NH₂ | H | CH₂S(C═ | H | | 59.6 | 1215 | 640 | 1862 | 640 | 880 |
| | | | | NH)NH₂ | | | | | | | | |
| 970 | H | CH₂N(Boc)C(═NBoc)NH₂ | H | H | H | Pinacol | 80 | 12 | nd | nd | nd | nd |
| | | | | | | ester | | | | | | |
| 973 | H | CH₂SC(═NH)NH₂ | H | H | OMe | pinacol | 32 | 1210 | 167 | 167 | 121 | 1210 |
| | | | | | | ester | | | | | | |
| 974 | H | CH(Me)SC(═NH)NH₂ | H | H | H | pinacol | 64 | 6769 | 324 | 680 | 324 | 934 |
| | | | | | | ester | | | | | | |
| 977 | H | CH₂SC(═NH)NH₂ | OPh | H | H | | 306 | 333 | 583 | 889 | 583 | 583 |
| 1006 | H | CH₂NHC(═NH)NH₂ | H | H | H | Pinacol | 46 | 106 | 37 | 51 | 27 | 70 |
| | | | | | | ester | | | | | | |
| 1030 | | CH₂S—C(═NH)NH₂ | Pip-N-Bz | | | | 5.9 | 12 | | | | |
| 1031 | | CH₂S—C(═NH)NH₂ | Pip-N (2-pyridyl) | | | | 39.0 | 15(*) | | | | |
| 1055 | | CH₂S—C(═NH)NH₂ | PhO | | | pinacol | 291 | 2768 | | | | |
| 1056 | | CH₂S—C(═NH)NH₂ | 4-PhO-PhO | | | pinacol | 626 | 455 | | | | |
| 1057 | | CH₂S—C(═NH)NH₂ | 3Cl-PhO | | | pinacol | 157 | 572 | | | | |
| 1058 | | CH₂S—C(═NH)NH₂ | 3MeOPhO | | | pinacol | 97 | 666 | | | | |
| 1059 | | CH₂S—C(═NH)NH₂ | 4-i-BuPhO | | | pinacol | 285 | 1959 | | | | |
| 1069 | | CH₂S—C(═NH)NH₂ | 3-CN-PhO | | | acid | 229 | 4156 | | | | |
| 1070 | | CH₂S—C(═NH)NH₂ | (4H)-β-napthylO | | | acid | 346 | 2174 | | | | |
| 1071 | | CH₂S—C(═NH)NH₂ | 2-(1,2-propenyl)- | | | acid | 287 | 1908 | | | | |
| | | | PhO | | | | | | | | | |
| 1072 | | CH₂S—C(═NH)NH₂ | PhCO-4-PhO | | | acid | 746 | 1410 | | | | |

Legend Table 3:
*Accuracy restricted by low solubility

1-69. (Canceled)

70. A pharmaceutical composition comprising a pharmaceutically acceptable diluent, excipient or carrier and a therapeutically effective amount of a compound of the formula: 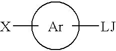
wherein,
Ar is an aryl or heteroaryl group independently selected from the group consisting of fused ring systems, partially aromatic systems, and rings, wherein Ar is optionally substituted by one or more moieties in addition to —X and -LJ;
X is selected from the group consisting of —CO₂E, —BY¹Y², and —CHO, wherein E is selected from the group consisting of H, an ester-forming group, and a cation;
Y¹ and Y² are independently selected from the group consisting of —OR¹, —SR¹, halogen, and —NR¹R²,
or Y¹ and Y² taken together with the boron atom to which they are attached form a cyclic boron ester (i.e. Y¹ and Y² together form a compound having two hydroxy groups), a cyclic boron amide (i.e. Y¹ and Y² together form the residue of a compound having two amino groups), or a cyclic boron amide-ester (i.e. Y¹ and Y² together form the residue of a compound having a hydroxy group and an amino group), and
wherein each R¹ and R² is independently selected from the group consisting of hydrogen and a moiety in which the non-hydrogen atoms are selected from the group consisting of C, N, O and S and number from 1 to 20 which comprises at least one hydrocarbyl group which may be aliphatic or carbocyclic and optionally 1, 2 or 3 heteroatoms selected from O, N and S;
L has the formula —(CR⁵R⁶)ₗ-(Z)ₘ—(CHR⁷)ₙ—, wherein R⁵, R⁶ and R⁷ are independently selected from the group consisting of —H, —OH, —NH₂, —SH, —NCCH₂CO₂(C₁-C₁₄)alkyl, and a moiety in which the atoms other than hydrogen and halogen are selected from the group consisting of C, N, O and S and number 1 to 20 and which comprises (i) at least one hydrocarbyl group, which is optionally substituted by halogen, and (ii) 0, 1, 2 or 3 heteroatoms selected from O, N and S;
wherein l is an integer from 0 to 6,
wherein Z is selected form the group consisting of O, N and S,
wherein m is an integer from 0 to 1,
wherein n is an integer from 0 to 6, and
wherein (l+m+n) is at least 1; and
J is selected from the group consisting of
(i) -GNR³R⁴,
(ii) -GNR¹OH,
(iii) -GNR¹C(NR¹)H,
(iv) -GNR¹C(NR¹)NR¹OH,
(v) -GNR¹C(NR¹)NR¹CN,
(vi) -GNR¹C(NR¹)NR¹COR¹,
(vii) -GNR¹C(NR¹)NR¹R²
(viii) -GC(NR¹)NR¹R²,
(ix) -GC(NR¹)NR¹NR¹COR¹,
(x) -GC(NR¹)NR¹C(NR¹)NR¹Re,
(xi) -GC(NR¹)NR¹COR¹,
(xii) -GNR¹C(O)R¹
(xiii) —OC(O)NR¹R²
(xiv) —OC(O)NR¹C(O)R¹,
(xv) —C(O)ONR³R⁴,
(xvi) -GN(R¹)COOR¹, and
(xvii) -GN(COOR¹)C(NH₂)═NCOOR¹,
wherein G is absent or is selected from the group consisting of SO₂, CO and CO(CH₂)ₚCO, wherein p is an integer selected form the group consisting of 1, 2, 3 and 4;
wherein R¹ and R² are as defined in claim 1 or claim 75 and each R¹ group of a compound having a plurality of R¹ groups is selected from the R¹ options independently of the other R¹ group(s) of the compound;
wherein each R¹ and R² is independently selected from the group consisting of hydrogen, C₁-C₁₀ alkyl optionally interrupted by an —O— linkage, C₅-C₁₀cyclohydrocarbyl, (C₁-C₄)alkyl-(C₅-C₁₀)cyclohydrocarbyl optionally interrupted by an —O— linkage or (C₅-C₁₀)cyclohydrocarbyl-(C₁-C₄)alkyl optionally interrupted by an —O— linkage, or C₅-C₆ cyclohydrocarbyl substituted by up to three groups selected from C₁-C₄ alkyl optionally interrupted by an linkage, C₁-C₄ alkoxy optionally interrupted by an —O— linkage and halogen, and a moiety in which the non-hydrogen atoms are selected from the group consisting of C, N, O and S and number from 1 to 20 which comprises at least one hydrocarbyl group which may be aliphatic or carbocyclic and optionally 1, 2 or 3 heteroatoms selected from O, N and S; wherein each R¹ group of a compound having a plurality of R¹ groups is optionally different;
wherein each R³ and R⁴ is independently selected from the group consisting of hydrogen, a moiety in which the atoms other than hydrogen and halogen are selected from the group consisting of C, N, O and S and number from 1 to 20 and which comprises at least one hydrocarbyl group which is optionally substituted by halogen and may be aliphatic or carbocyclic and optionally 1, 2 or 3 heteroatoms selected from O, N and S, or R³ and R⁴ together with their attached N form a ring, which is optionally part of a fused ring system and/or substituted by C₁-C₁₀ alkyl, a C₁-C₁₀ alkyl moiety substituted by carboxyl, alkoxycarbonyl (wherein the alkoxycarbonylalkyl group contains from 1 to 12 carbon atoms), alkoxy (wherein the alkoxylalkyl group contains from 1 to 12 carbon atoms), hydroxy or halogen; (C₁-C₄)alkyl-(C₅-C₁₀)cyclohydrocarbyl; C₅-C₁₀ cyclohydrocarbyl, which is optionally substituted by 1, 2 or 3 moieties selected from the group consisting of 5- and 6-membered rings, hydroxy, amino, C₁, C₂, C₃ or C₄ alkyl, C₁, C₂, C₃ or C₄ alkoxy, thiol, C₁, C₂, C₃ or C₄ alkylthio, amino, nitrile, carboxy, —CHO, —C(O)alkyl, —SO₂, substituted amino and halogen, the aforesaid organic substituents optionally being substituted by halogen and the aforesaid alkyl-, cyclohydrocarbyl- and ring-containing substituents optionally being terminated by an ether or thioether linkage to the moiety which they substitute and/or, in the case of substituents containing at least one alkylic carbon atom, interrupted at an alkylic carbon by an ether or thioether linkage; or, -L-J is of the formula —C═N—NR¹—C(NR¹)NR¹R²,
wherein the compound additionally includes all variant forms of these compounds, for example tautomers; salts, addition salts, esters, acids and their tautomers; species which are capable of existing in equilibrium with any of the aforesaid; and species which, upon administration, are capable of providing directly or indirectly any of the aforesaid.

71. The composition of claim 70, wherein said species that are capable of existing in equilibrium contain a tetrahedral boronic moiety.

72. The composition of claim 70, wherein Ar contains from 5 to 13 ring-forming atoms and, when a ring, is a 5- or 6-membered ring.

73. A composition of claim 70, wherein Ar wherein Ar is phenyl.

74. The composition of claim 70, wherein X is —BY¹Y².

75. The composition of claim 74, wherein R¹ and R² are independently selected from the group consisting of H, C₁-C₁₀ alkyl optionally interrupted by an —O— linkage, C₅-C₁₀cyclohydrocarbyl, (C₁-C₄)alkyl-(C₅-C₁₀)cyclohydrocarbyl optionally interrupted by an —O— linkage or (C₅-C₁₀)cyclohydrocarbyl-(C₁-C₄)alkyl optionally interrupted by an —O— linkage, or C₅-C₆ cyclohydrocarbyl substituted by up to three groups selected from C₁-C₄ alkyl optionally interrupted by an —O— linkage, C₁-C₄ alkoxy optionally interrupted by an —O— linkage and halogen,
or —BY¹Y² is a moiety convertible in vivo into —B(OH)₂.

76. The composition of claim 75, wherein Y¹ and Y² are independently selected from the group consisting of hydroxy and C₁-C₁₀ alkoxy wherein alkyl portion is optionally interrupted by an ether linkage,
wherein Y¹ and Y² optionally form a cyclic boron ester together with the boron atom to which they are joined.

77. The composition of claim 76, wherein Y¹ and Y² together with the boron atom form a cyclic boron ester which is formed by the boron atom together with the residue of pinacol, pinanediol, neopentylglycol, diethanolamine, 1,2-ethanediol, 1,2-propanediol, 1,3-propanediol, 2,3-butanediol, 1,2-diisopropylethanediol, 5,6-decanediol or 1,2-dicyclohexylethanediol.

78. The composition of claim 70, wherein l is an integer from 0 to 2; m is 1; and n is an integer form 0 to 2.

79. The composition of claim 78, wherein l is 1 and n is 0.

80. The composition of claim 70, wherein R⁵, R⁶ and R⁷ are independently selected from hydrogen and an optionally substituted moiety selected from the group consisting of C₁-C₈ alkyl, C₁-C₈ alkenyl, C₁-C₈ alkynyl, C₅-C₁₀cyclohydrocarbyl, (C₁-C₄)alkyl-(C₅-C₁₀)cyclohydrocarbyl, (C₅-C₁₀)cyclohydrocarbyl-(C₁-C₄)alkyl and C₅-C₁₀ cyclohydrocarbyl and said moieties when terminated by an ether, thioether or amino (—NH—) linkage to the remainder of L and/or, in the case of moieties containing at least one alkylic carbon atom, interrupted at an alkylic carbon atom by a said linkage, the optional substitution being by halogen or by an OH, SH or NH₂ group.

81. The composition of claim 80, wherein each R⁵ and R⁶ is independently H, C₁-C₈ alkyl, phenyl, C₁-C₈ alkoxy, C₁-C₈ alkythio, phenoxy or phenylthio; and R⁷ is H or C₁-C₈ alkyl.

82. The composition of claim 81, wherein each R⁵ and R⁶ is independently selected from H, C₁-C₈ alkyl and phenyl.

83. The composition of claim 82, wherein each R⁵ and R⁶ is independently H or methyl.

84. The composition claim 70, wherein the total of R⁵ and R⁶ groups which is other than hydrogen is 0, 1 or 2.

85. The composition claim 84, wherein the total is 0 or 1.

86. The composition claim 70, wherein there is no more than one R⁷ group which is other than hydrogen.

87. The composition claim 84, wherein the total of (R⁵+R⁶+R⁷) groups which is other than hydrogen is 0 or 1.

88. The composition of claim 70, wherein Z is S.

89. The composition of claim 70, wherein —(CR⁵R⁶)ₗ— is —CH²— or —CHalkyl- wherein said CHalkyl contains from 1 to 9 carbon atoms.

90. The composition of claim 70, wherein CHR⁷)ₙ— is —CH₂— or —CHalkyl-, wherein said CHalkyl contains from 1 to 9 carbon atoms.

91. The composition of claim 70, wherein -L-J is —CHR⁶—S-J, wherein R⁶ is H or C₁-C₈ alkyl.

92. The composition of claim 70, wherein J is -GNR³R⁴, —C(O)ONR³R⁴, -GC(NR¹)NR¹R² or -GC(NR¹)NR¹C(NR¹)N—R¹R².

93. The composition of claim 92 wherein G is absent.

94. The composition of claim 70, wherein each R³ and R⁴ is independently selected from the group consisting of C₁-C₁₀ alkyl; C₁-C₁₀ alkyl substituted by carboxyl; alkoxycarbonyl wherein the alkoxycarbonylalkyl group contains from 1 to 12 carbon atoms; alkoxy wherein the alkoxylalkyl group contains from 1 to 12 carbon atoms; hydroxy; halogen; C₅-C₁₀ cyclohydrocarbyl optionally substituted with a substituent selected from group consisting of C₁-C₄ alkyl, C₁-C₄ alkoxy and halogen; (C₅-C₁₀)cyclohydrocarbyl-(C₁-C₄)alkyl whose cyclohydrocarbyl part is optionally substituted by a substituent selected from the group consisting of C₁-C₄ alkyl, C₁-C₄ alkoxy and halogen; and C₅-C₆ cycloalkyl substituted by two or three groups selected from C₁-C₄ alkyl, C₁-C₄ alkoxy and halogen;
wherein R³ and R⁴ together optionally form a ring as specified in claim 70.

95. The composition of claim 70, wherein J comprises a heterocycle having a pKa of from about 7 to about 9.

96. The composition of claim 95, wherein J comprises a ring structure selected from the group consisting of an imidazole, a benzoxazole, a benzimidazole ring, and a ring comprising a cyclic residue wherein said cyclic residue is selected from the group consisting of 2,4,6-trimethylpyridinyl, 3-hydroxyquinolinyl, 5-hydroxyquiazolinyl and morpholinyl.

97. The composition of claim 96, wherein the ring structure is substituted by a ring substituent listed in claim 70.

98. The composition of claim 97, wherein the heterocycle having a pka of from about 7 to 9 is formed by R³ and R⁴ together with their attached N.

99. The composition of claim 70, wherein each R¹ is H.

100. The composition of claim 70, wherein J is selected from the group consisting of -GNH₂, -GNHalkyl, -GNHCH₂carboxyalkyl, -GN(alkyl)₂ and a moiety of the following structure: 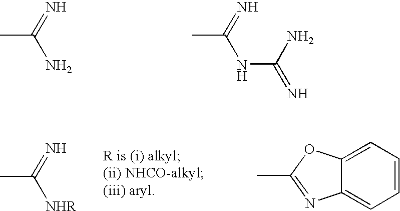
wherein “alkyl” and “alkoxy” have 1, 2, 3, 4, 5 or 6 carbon atoms, and “aryl” is phenyl or phenyl substituted by C₁, C₂, C₃ or C₄ alkyl or C₁, C₂, C₃ or C₄ alkoxy.

101. The composition of claim 70, wherein Ar is additionally substituted by 0 to 4 further substituents selected from the group consisting of -LJ moieties, X moieties, J moieties, -linker-RING, -linker-RING-linker-RING, -linker-D, halogen, hydroxy, a hydroxy derivative, thiol, alkylthio, amino, nitrile, carboxy, —CHO, —C(O)alkyl, —SO₂, and substituted amino, and moieties containing from 1 to 30 carbon atoms and comprising at least one group selected from substituted or unsubstituted aliphatic, alicyclic or (hetero)aromatic groups, substituents for the aliphatic, alicyclic and (hetero)aromatic moieties being selected from halogen, hydroxy, a hydroxy derivative, thiol, alkylthio, amino, nitrile, carboxy, —CHO, —C(O)alkyl, —SO₂, mono- or di-alkylamino or alkylamido, and the alkyl groups, or the alkyl part of alkyl-containing moieties, containing from 1 to 10 carbon atoms,
wherein linker is selected from the group consisting of a bond, —O—, —NH—, —NH-M-, -M- and C, C₂, C₃ or C₄ alkylene optionally interrupted or terminated by one or more —O— or —NH— linkages, where M is SO₂, CO, (CH²)_{q}SO₂, (CH₂)_{q}CO, CO(CH₂)_{q}CO or CHMeCO, where q is 1, 2, 3, or 4,
wherein each RING is a mono- or bi-cyclic ring additionally substituted by 0 to 3 substituents selected from the group consisting of halogen, hydroxy, a hydroxy derivative, thiol, alkylthio, amino, nitrile, carboxy, —CHO, —C(O)alkyl, —SO₂R¹ (where R¹ is as defined in claim 70), —SO₂aa (where aa is a natural or unnatural amino acid), mono- or di-alkylamino and alkylamido; the aforesaid alkyl groups containing from 1 to 10 carbon atoms and optionally being interrupted by one or a plurality of ether linkages and/or substituted by halogen, and
wherein D is a C₁-C₁₆ moiety constituted by alkyl, alkylene, cycloalkyl and/or cycloalkylene residues wherein the alkyl and alkylene residues may be interrupted by, or linked to an adjacent residue by, an —O— linkage or be substituted by —CHO, —SO₂R, or a residue of a natural or unnatural amino acid.

102. The composition of claim 101, wherein Ar is additionally substituted by a single further substituent Q which is at the 2- or 4-position to X, contains up to 20 carbon atoms and is selected from the group consisting of: H, X moieties (in which case the two X moieties of the compound may be the same or different), J moieties (in which case the two J moieties of the compound may be the same or different), -linker-RING (as defined in claim 101), -linker-RING-linker-RING, halogen, hydroxy, hydroxy derivatives, thiol, alkylthio, substituted or unsubstituted aliphatic, alicyclic or (hetero)aromatic groups, and substituted or unsubstituted moieties containing at least two groups selected from aliphatic, alicyclic and (hetero)aromatic groups and optionally one or more M groups (as defined in claim 101) interconnecting adjacent ones of said at least two groups, the substituents for the aliphatic, alicyclic and (hetero)aromatic moieties being selected from halogen, hydroxy, a hydroxy derivative, thiol, alkylthio, amino, nitrile, carboxy, —CHO, —C(O)alkyl, —SO₂, mono- or di-alkylamino and alkylamido, and the alkyl groups containing from 1 to 10 carbon atoms unless otherwise indicated.

103. The composition of claim 70, wherein -LJ is at the 3-position to X.

104. The composition of claim 70 having the formula III, IV or V 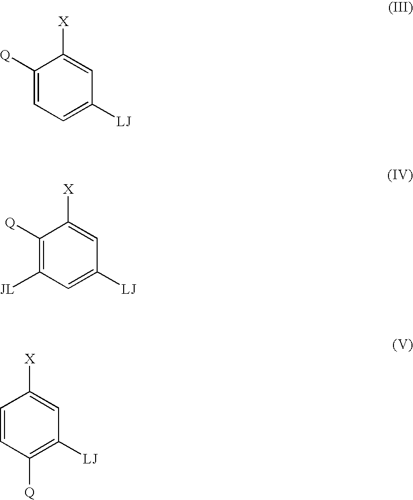
or variants thereof in which phenyl is replaced by a wholly or partially hydrogenated analogue thereof, wherein Q is as defined in claim 102.

105. The composition of claim 104, wherein Q is selected from the group consisting of H, -linker-RING, -linker-RING-linker-RING, or -linker-D.

106. The composition of claim 105, wherein Q is -linker-RING-linker-RING, where each linker independently selected from the group consisting of a bond, —O—, —NH—, —NH—CO—, —CO— and each RING is a monocyclic ring.

107. The composition of claim 104, wherein Q is H or 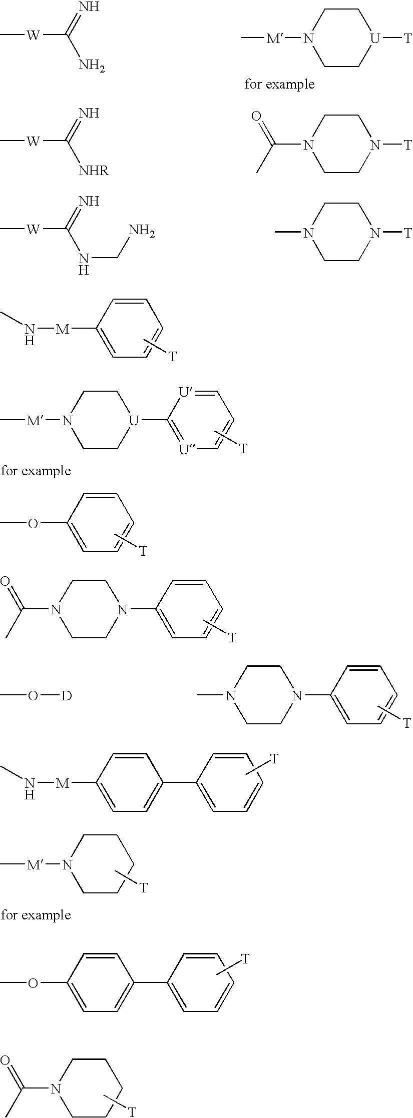
wherein:
W is absent or —CHR¹—S—, wherein R¹ is H or C₁-C₁₀ alkyl;
R is C₁-C₁₀ alkyl, —NHCO—(C₁-C₁₀)alkyl, or C₅-C₁₀cyclohydrocarbyl;
M is M is SO₂, CO, (CH²)_{q}SO₂, (CH₂)_{q}CO, CO(CH₂) or CHMeCO, where q is 1, 2, 3, or 4;
T is H, C₁-C₁₀ alkyl, C₁-C₁₀ alkoxy, —CHO, —SO₂R, halo, nitrile or a residue of a natural or unnatural amino acid;
D is a C₁-C₁₆ moiety constituted by alkyl, alkylene, cycloalkyl and/or cycloalkylene residues wherein the alkyl and alkylene residues may be interrupted by, or linked to an adjacent residue by, an —O— linkage or be substituted by —CHO, —SO₂R, or a residue of a natural or unnatural amino acid;
M′ is nothing or an M group;
U, U′ and U″ are each independently C or N and, if C, are optionally substituted by C₁-C₁₀ alkyl or C₁-C₁₀ alkoxy independently of the substitution status of the others of U, U′ and U″.

108. The composition of claim 104, wherein Q is H and each -LJ group is of the formula
—CH₂—S-J¹ or —CHMe—S-J¹,
wherein J¹ is selected from the group consisting of —C(NH)NH₂ (amidino), —NHC(NH)NH₂ (guanidino), —NH₂ (amino), —C(O)NH₂ (carboxamido), —NH₂OH (hydroxylamino), or imidazolyl or an N-substituted analogue thereof in which there is an N-substituent selected from C₁-C₈ alkyl, C₅-C₆ cycloalkyl and phenyl.

109. The composition of claim 70, wherein -L-J is selected from the group consisting of —(CR⁵R⁶), —S_(CHR⁷)ₙ—C(NH)NHR″, wherein R⁵, R⁶, R⁷ and l are as defined in claim 80, R″ is H, C₁-C₈ alkyl, C₅-C₆ cycloalkyl or phenyl, and X is BY¹Y² as defined in any of claims 70 or 75 to 77.

110. The composition of claim 109, wherein -L-J is —(CR⁵R⁶)₁—S—C(NH)NHR″.

111. The composition of claim 109, wherein Ar comprises phenyl or a wholly or partially hydrogenated analogue thereof and BY¹Y² is in a 1,4 or 1,3 relationship with the -L-J group.

112. The composition of claim 109, wherein the compound is of formula (VII): 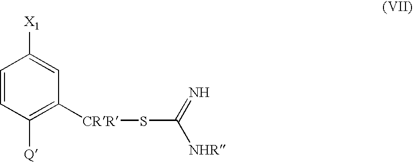
or variants thereof in which the benzene ring is replaced by a wholly or partially hydrogenated analogue thereof,
wherein:
X₁ is —B(OR′″)₂, or a cyclic boron ester wherein R′″ is H or C₁-C₈ alkyl;
each R¹ is independently selected from the group consisting of H and C₁-C₈ alkyl;
R″ is selected from the group consisting of H, C₁-C₈ alkyl, C₅-C₆ cycloalkyl and phenyl; and
Q′ is H, C₁-C₁₀ alkyl, C₁-C₁₀ alkoxyalkyl, C₁-C₁₀ alkoxy, C₆-C₁₀ aryloxy, C₅-C₆ cycloalkyloxy, C₆-C₁₀ aryloxyaryl, wholly or partially hydrogenated analogues of C₁-C₈ aryl or C₁-C₈ aryloxyaryl, the aryl moieties and their hydrogenated analogues being substituted by 0, 1, 2 or 3 substituents selected from halogen, C₁-C₈ alkyl, hydroxy, C₁-C₈ alkoxy, thiol, C₁-C₈ alkylthio, amino, nitrile, carboxy, —CHO, —C(O)C₁-C₈ alkyl, —SO₂R¹ (where R¹ is as defined previously), and mono- or di-(C₁-C₈)alkylamino; the aforesaid alkyl groups (including the alkyl part of alkyl-containing moieties) optionally being interrupted by one or a plurality of ether linkages, or: 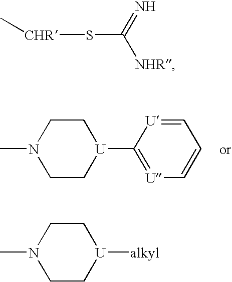
wherein each U, U′ and U″ is independently selected from the group consisignt of C and N wherein, if C, are optionally substituted by C₁-C₈ alkyl or C₁-C₈ alkoxy independently of the substitution status of the others of U, U′ and U″, and R′ and R″ are as defined above.

113. The composition of claim 109, wherein the compound is of Formula (XIV): 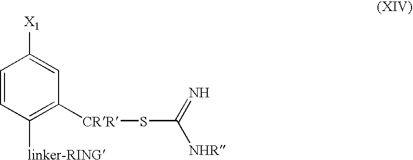
or variants thereof in which phenyl is replaced by a wholly or partially hydrogenated analogue thereof,
wherein X₁, R′ and R″ are as defined in claim 112, linker is as defined in claim 101, and RING′ is a mono- or bi-cyclic ring substituted, through a “linker” as defined in claim 101, by a second mono- or bi-cyclic ring, each mono- or bi-cyclic ring being further substituted by 0, 1, 2 or 3 substituents selected from halogen, alkyl, hydroxy, alkoxy, thiol, alkylthio, amino, nitrile, carboxy, —CHO, —C(O)alkyl, —SO₂R¹ where R¹ is as defined in claim 70, mono- or di-alkylamino and alkylamido; the aforesaid alkyl groups including the alkyl part of alkyl-containing moieties containing from 1 to 10 carbon atoms and optionally being interrupted by one or a plurality of ether linkages.

114. The composition of claim 109, wherein the compound is of formula (XIIIA) or (XIIIB): 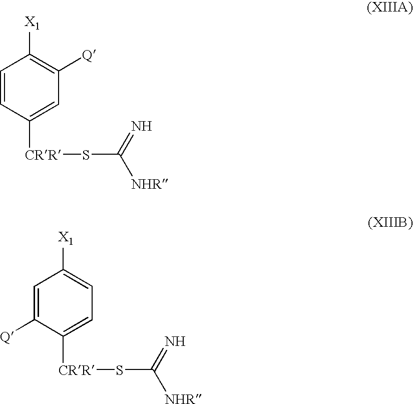
or variants thereof in which the benzene ring is replaced by a wholly or partially hydrogenated analogue thereof, wherein X₁, Q′, R′ and R″ are as defined in claim 112.

115. A method of inhibiting a serine protease in the treatment of disease comprising administering to a mammal a therapeutically effective amount of a compound as defined in claim 70.

116. A method of inhibiting bacterial sporulation, comprising applying a compound as defined in claim 70 to a substrate believed to harbour bacteria.

117. An affinity chromatography column, which comprises a compound as defined in claim 70 bound to a solid phase.

118. A method of inhibiting Factor IXa, Factor Xa or thrombin in the treatment of disease comprising administering to a mammal a therapeutically effective amount of a compound as defined in claim 1.

119. A method of inhibiting Factor IXa in the treatment of disease comprising administering to a mammal a therapeutically effective amount of a compound of Formula (XIV): 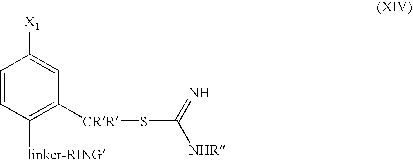
or variants thereof in which phenyl is replaced by a wholly or partially hydrogenated analogue thereof,
wherein X₁, R′ and R″ are as defined in claim 112, linker is as defined in claim 101, and RING′ is a mono- or bi-cyclic ring substituted, through a “linker” as as defined in claim 101, by a second mono- or bi-cyclic ring, each mono- or bi-cyclic ring being further substituted by 0, 1, 2 or 3 substituents selected from halogen, alkyl, hydroxy, alkoxy, thiol, alkylthio, amino, nitrile, carboxy, —CHO, —C(O)alkyl, —SO₂R¹ (where R¹ is as defined in claim 1), mono- or di-alkylamino and alkylamido; the aforesaid alkyl groups (including the alkyl part of alkyl-containing moieties) containing from 1 to 10 carbon atoms and optionally being interrupted by one or a plurality of ether linkages.

120. A method of inhibiting urokinase in the treatment of disease comprising administering to a mammal a therapeutically effective amount of a compound of formula (XIIIA) or (XIIIB) 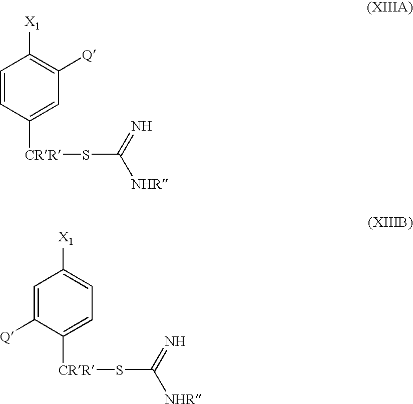
or variants thereof wherein the benzene ring is replaced by a wholly or partially hydrogenated analogue thereof, and wherein X₁, Q′, R′ and R″ are as defined in claim 112.

121. The composition of claim 70, wherein X is —BY¹Y² and Ar is phenyl.

122. The composition of claim 121, wherein-L-J is —CHR⁶—S-J, wherein R⁶ is H or C₁-C₈ alkyl.

123. The composition of claim 101, wherein X is —BY¹Y².

124. A pharmaceutical composition comprising a pharmaceutically acceptable diluent, excipient or carrier and a therapeutically effective amount of a compound of the formula: 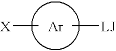
wherein:
Ar is a cyclic moiety selected from the group consisting of (i) 6-membered rings and (ii) fused rings comprising a ring selected from phenyl and wholly and partially hydrogenated analogues of phenyl, and Ar may be substituted by one or more moieties in addition to —X and -LJ;
X is a functional group which is hydrogen-bond acceptor or a group transformed in vivo into a hydrogen bond acceptor;
Y¹ and Y² are each independently selected from the group consisting of —OR¹, —SR¹, halogen and —NR¹R²,
or Y¹ and Y² taken together with the boron atom to which they are attached form a cyclic boron ester (i.e. Y¹ and Y² together form a compound having two hydroxy groups), a cyclic boron amide (i.e. Y¹ and Y² together form the residue of a compound having two amino groups), or a cyclic boron amide-ester (i.e. Y¹ and Y² together form the residue of a compound having a hydroxy group and an amino group),
wherein each R¹ and R² is independently selected form the group consisting of hydrogen and a moiety in which the non-hydrogen atoms are selected from the group consisting of C, N, O and S and number from 1 to 20 which comprises at least one hydrocarbyl group which may be aliphatic or carbocyclic and optionally 1, 2 or 3 heteroatoms selected from O, N and S;
L is of the formula —(CR⁵R⁶)ₗ-(Z)ₘ—(CHR⁷)ₙ—,
wherein R⁵, R⁶ and R⁷ are independently selected from the group consisting of —H, —OH, —NH₂, —SH, —NCCH₂CO₂(C₁-C₁₄)alkyl and a moiety in which the atoms other than hydrogen and halogen are selected from the group consisting of C, N, O and S and number 1 to 20 and which comprises (i) at least one hydrocarbyl group, which is optionally substituted by halogen, and (ii) 0, 1, 2 or 3 heteroatoms selected from O, N and S,
wherein l is an integer from 0 to 6,
wherein Z is O, N or S,
wherein m is an integer from 0 to 1;
wherein n is an integer from 0 to 6, and
wherein (l+m+n) is at least 1; and
J is selected from the group consisting of
(i) -GNR³R⁴,
(ii) -GNR¹OH,
(iii) -GNR¹C(NR¹)H,
(iv) -GNR¹C(NR¹)NR¹OH,
(v) -GNR¹C(NR¹)NR¹CN,
(vi) -GNR¹C(NR¹)NR¹COR¹,
(vii) -G′NR¹C(NR¹)NR¹R²
(viii) -GC(NR¹)NR¹R²,
(ix) -GC(NR¹)NR¹NR¹COR¹,
(x) -GC(NR¹)NR¹C(NR¹)NR¹R²,
(xi) -GC(NR¹)NR¹COR¹,
(xii) -GNR¹C(O)R¹
(xiii) —OC(O)NR¹R²
(xiv) —OC(O)NR¹C(O)R¹,
(xv) —C(O)ONR³R⁴,
(xvi) -GN(R¹)COOR¹, and
(xvii) -GN(COOR¹)C(NH₂)═NCOOR¹,
wherein G is absent or is SO₂, CO or CO(CH₂)ₚCO, wherein p is 1, 2, 3 or 4, G′ is SO₂, CO or CO(CH₂)ₚCO, wherein p is 1, 2, 3 or 4,
wherein R¹ and R² are as defined in claim 1 or claim 75 and each R¹ group of a compound having a plurality of R¹ groups is selected from the R¹ options independently of the other R¹ group(s) of the compound;
wherein each R³ and R⁴ is independently selected from the group consisting of hydrogen, a moiety in which the atoms other than hydrogen and halogen are selected from the group consisting of C, N, O and S and number from 1 to 20 and which comprises at least one hydrocarbyl group which is optionally substituted by halogen and may be aliphatic or carbocyclic and optionally 1, 2 or 3 heteroatoms selected from O, N and S, or R³ and R⁴ together with their attached N form a ring, which is optionally part of a fused ring system and/or substituted by C₁-C₁₀ alkyl, a C₁-C₁₀ alkyl moiety substituted by carboxyl, alkoxycarbonyl wherein the alkoxycarbonylalkyl group contains from 1 to 12 carbon atoms, alkoxy wherein the alkoxylalkyl group contains from 1 to 12 carbon atoms, hydroxy or halogen; (C₁-C₄)alkyl-(C₅-C₁₀)cyclohydrocarbyl; C₅-C₁₀ cyclohydrocarbyl, which is optionally substituted by 1, 2 or 3 moieties selected from the group consisting of 5- and 6-membered rings, hydroxy, amino, C₁, C₂, C₃ or C₄ alkyl, C₁, C₂, C₃ or C₄ alkoxy, thiol, C₁, C₂, C₃ or C₄ alkylthio, amino, nitrile, carboxy, —CHO, —C(O)alkyl, —SO₂, substituted amino and halogen, the aforesaid organic substituents optionally being substituted by halogen and the aforesaid alkyl-, cyclohydrocarbyl- and ring-containing substituents optionally being terminated by an ether or thioether linkage to the moiety which they substitute and/or, in the case of substituents containing at least one alkylic carbon atom, interrupted at an alkylic carbon by an ether or thioether linkage; or, -L-J is of the formula —C═N—NR¹—C(NR¹)NR¹R²; and
wherein the compound additionally comprises all variant forms of these compounds, for example tautomers; salts, addition salts, esters, acids and their tautomers; species which are capable of existing in equilibrium with any of the aforesaid; and species which, upon administration, are capable of providing directly or indirectly any of the aforesaid.

125. The composition of claim 101, wherein X is —BY¹Y².

126. A compound of the formula: 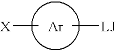
wherein:
Ar is a cyclic moiety selected from the group consisting of 6-membered rings and fused rings and may be substituted by one or more moieties in addition to —X and -LJ;
X is —BY¹Y² or —CN;
Y¹ and Y² are independently selected from the group consisting of —OR¹, —SR¹, halogen and —NR¹R², Y¹ and Y² taken together with the boron atom to which they are attached form a cyclic boron ester (i.e. Y¹ and Y² together form a compound having two hydroxy groups), a cyclic boron amide (i.e. Y¹ and Y² together form the residue of a compound having two amino groups), or a cyclic boron amide-ester (i.e. Y¹ and Y² together form the residue of a compound having a hydroxy group and an amino group),
wherein each R¹ and R² is independently selected from the group consisting of hydrogen and a moiety in which the non-hydrogen atoms are selected from the group consisting of C, N, O and S and number from 1 to 20 which comprises at least one hydrocarbyl group which may be aliphatic or carbocyclic and optionally 1, 2 or 3 heteroatoms selected from O, N and S;
L is of the formula —(CR⁵R⁶)ₗ-(Z)ₘ—(CHR⁷)ₙ—,
wherein R⁵, R⁶ and R⁷ are independently selected from the group consisting of is —H, —OH, —NH₂, —SH, —NCCH₂CO₂(C₁-C₁₄)alkyl and a moiety in which the atoms other than hydrogen and halogen are selected from the group consisting of C, N, O and S and number 1 to 20 and which comprises (i) at least one hydrocarbyl group, which is optionally substituted by halogen, and (ii) 0, 1, 2 or 3 heteroatoms selected from O, N and S;
wherein l is an integer from 0 to 6;
wherein Z is O, N or S;
wherein m is an integer from 0 to 1;
wherein n is an integer from 0 to 6;
wherein (1+m+n) is at least 1; and
J is selected from the group consisting of
(i) -GNR³R⁴,
(ii) -GNR¹OH,
(iii) -GNR¹C(NR¹)H,
(iv) -GNR¹C(NR¹)NR¹OH,
(v) -GNR C(NR¹)NR¹CN,
(vi) -GNR C(O)NR¹COR¹,
(vii) -GNR¹C(NR¹)NR¹R²
(viii) -GC(NR¹)NR¹R²,
(ix) -GC(NR¹)NR¹NR¹COR¹,
(x) -GC(NR¹)NR¹C(NR¹)NR¹R²,
(xi) -GC(NR¹)NR¹COR¹,
(xii) -GNR¹C(O)R¹
(xiii) —OC(O)NR¹R²
(xiv) —OC(O)NR¹C(O)R¹,
(xv) —C(O)ONR³R⁴,
(xvi) -GN(R¹)COOR¹, and
(xvii) -GN(COOR¹)C(NH₂)═NCOOR¹,
wherein G is absent or is SO₂, CO or CO(CH₂)ₚCO, wherein p is 1, 2, 3 or 4,
wherein G′ is SO₂, CO or CO(CH₂)ₚCO, wherein p is 1, 2, 3 or 4,
wherein R¹ and R² are as defined in claim 1 or claim 75 and each R¹ group of a compound having a plurality of R¹ groups is selected from the R¹ options independently of the other R¹ group(s) of the compound,
wherein each R³ and R⁴ is independently selected from hydrogen, a moiety in which the atoms other than hydrogen and halogen are selected from the group consisting of C, N, O and S and number from 1 to 20 and which comprises at least one hydrocarbyl group which is optionally substituted by halogen and may be aliphatic or carbocyclic and optionally 1, 2 or 3 heteroatoms selected from O, N and S, or R³ and R⁴ together with their attached N form a ring, which is optionally part of a fused ring system and/or substituted by C₁-C₁₀ alkyl, a C₁-C₁₀ alkyl moiety substituted by carboxyl, alkoxycarbonyl (wherein the alkoxycarbonylalkyl group contains from 1 to 12 carbon atoms), alkoxy (wherein the alkoxylalkyl group contains from 1 to 12 carbon atoms), hydroxy or halogen; (C₁-C₄)alkyl-(C₅-C₁₀)cyclohydrocarbyl; C₅-C₁₀ cyclohydrocarbyl, which is optionally substituted by 1, 2 or 3 moieties selected from the group consisting of 5- and 6-membered rings, hydroxy, amino, C₁, C₂, C₃ or C₄ alkyl, C₁, C₂, C₃ or C₄ alkoxy, thiol, C₁, C₂, C₃ or C₄ alkylthio, amino, nitrile, carboxy, —CHO, —C(O)alkyl, —SO₂, substituted amino and halogen, the aforesaid organic substituents optionally being substituted by halogen and the aforesaid alkyl-, cyclohydrocarbyl- and ring-containing substituents optionally being terminated by an ether or thioether linkage to the moiety which they substitute and/or, in the case of substituents containing at least one alkylic carbon atom, interrupted at an alkylic carbon by an ether or thioether linkage; or, -L-J is of the formula —C═N—NR¹—C(NR¹)NR¹R²,
wherein the compound additionally comprises all variant forms of these compounds, for example tautomers; salts, addition salts, esters, acids and their tautomers; species which are capable of existing in equilibrium with any of the aforesaid; and species which, upon administration, are capable of providing directly or indirectly any of the aforesaid,
with the proviso that the compound is not a benzeneboronic acid derivative of the formula 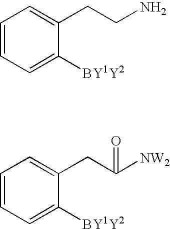
wherein
each W is H, or
one W is H and the other is the residue of 2-aminophenol, or
both W groups together with the adjoining N form piperidine, and
Y¹ and Y² are both —OH or together form the residue of catechol.

127. A compound of the formula: 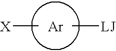
wherein
Ar is a ring or ring system, which may be at least partially aromatic but can be non-aromatic and may be substituted by one or more moieties in addition to —X and -LJ;
X is —BY¹Y², wherein Y¹ and Y² are independently selected from the group consisting of —OR¹, —SR¹, halogen or —NR¹R²,
or Y¹ and Y² taken together with the boron atom to which they are attached form a cyclic boron ester (i.e. Y¹ and Y² together form a compound having two hydroxy groups), a cyclic boron amide (i.e. Y¹ and Y² together form the residue of a compound having two amino groups), or a cyclic boron amide-ester (i.e. Y¹ and Y² together form the residue of a compound having a hydroxy group and an amino group),
wherein each R¹ and R² is independently selected from the group consisting of hydrogen and a moiety in which the non-hydrogen atoms are selected from the group consisting of C, N, O and S and number from 1 to 20 which comprises at least one hydrocarbyl group which may be aliphatic or carbocyclic and optionally 1, 2 or 3 heteroatoms selected from O, N and S;
L is of the formula —(CR⁵R⁶)1-(Z)ₘ—(CHR⁷)ₙ—,
wherein R⁵, R⁶ and R⁷ are independently selected from the group consisting of is —H, —OH, —NH₂, —SH, —NCCH₂CO₂(C₁-C₁₄)alkyl and a moiety in which the atoms other than hydrogen and halogen are selected from the group consisting of C, N, O and S and number 1 to 20 and which comprises (i) at least one hydrocarbyl group, which is optionally substituted by halogen, and (ii) 0, 1, 2 or 3 heteroatoms selected from O, N and S;
wherein l is an integer from 0 to 6;
wherein Z is O, N or S;
wherein m is an integer from 0 to 1;
wherein n is an integer from 0 to 6;
wherein (l+m+n) is at least 1; and
J is selected from the group consisting of
(i) -GNR³R⁴,
(ii) -GNR¹OH,
(iii) -GNR¹C(NR¹)H,
(iv) -GNR¹C(NR¹)NR¹OH,
(v) -GNR¹C(NR¹)NR¹CN,
(vi) -GNR¹C(NR¹)NR¹COR¹,
(vii) -G′NR¹C(NR¹)NR R²
(viii) -GC(NR¹)NR¹R²,
(ix) -GC(NR¹)NR¹NR¹COR¹,
(x) -GC(NR¹)NR¹C(NR¹)NR¹R²,
(xi) -GC(NR¹)NR¹COR¹,
(xii) -GNR¹C(O)R¹
(xiii) —OC(O)NR¹R²
(xiv) —OC(O)NR¹C(O)R¹,
(xv) —C(O)ONR³R⁴,
(xvi) -GN(R¹)COOR¹, and
(xvii) -GN(COOR¹)C(NH₂)═NCOOR¹,
wherein G is absent or is SO₂, CO or CO(CH₂)ₚCO, wherein p is 1, 2, 3 or 4,
wherein G′ is SO₂, CO or CO(CH₂)ₚCO, wherein p is 1, 2, 3 or 4,
wherein R¹ and R² are as defined in claim 1 or claim 75 and each R¹ group of a compound having a plurality of R¹ groups is selected from the R¹ options independently of the other R¹ group(s) of the compound,
wherein each R³ and R⁴ is independently selected from hydrogen, a moiety in which the atoms other than hydrogen and halogen are selected from the group consisting of C, N, O and S and number from 1 to 20 and which comprises at least one hydrocarbyl group which is optionally substituted by halogen and may be aliphatic or carbocyclic and optionally 1, 2 or 3 heteroatoms selected from O, N and S, or R³ and R⁴ together with their attached N form a ring, which is optionally part of a fused ring system and/or substituted by C₁-C₁₀ alkyl, a C₁-C₁₀ alkyl moiety substituted by carboxyl, alkoxycarbonyl (wherein the alkoxycarbonylalkyl group contains from 1 to 12 carbon atoms), alkoxy (wherein the alkoxylalkyl group contains from 1 to 12 carbon atoms), hydroxy or halogen; (C₁-C₄)alkyl-(C₅-C₁₀)cyclohydrocarbyl; C₅-C₁₀ cyclohydrocarbyl, which is optionally substituted by 1, 2 or 3 moieties selected from the group consisting of 5- and 6-membered rings, hydroxy, amino, C₁, C₂, C₃ or C₄ alkyl, C₁, C₂, C₃ or C₄ alkoxy, thiol, C₁, C₂, C₃ or C₄ alkylthio, amino, nitrile, carboxy, —CHO, —C(O)alkyl, —SO₂, substituted amino and halogen, the aforesaid organic substituents optionally being substituted by halogen and the aforesaid alkyl-, cyclohydrocarbyl- and ring-containing substituents optionally being terminated by an ether or thioether linkage to the moiety which they substitute and/or, in the case of substituents containing at least one alkylic carbon atom, interrupted at an alkylic carbon by an ether or thioether linkage; or, -L-J is of the formula —C═N—NR¹—C(NR¹)NR¹R²,
wherein the compound additionally comprises all variant forms of these compounds, for example tautomers; salts, addition salts, esters, acids and their tautomers; species which are capable of existing in equilibrium with any of the aforesaid; and species which, upon administration, are capable of providing directly or indirectly any of the aforesaid,
with the proviso that the compound is not a benzeneboronic acid derivative of the formula 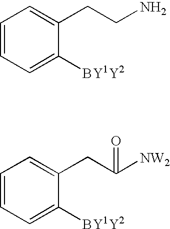
wherein
each W is H, or
one W is H and the other is the residue of 2-aminophenol, or
both W groups together with the adjoining N form piperidine, and
Y¹ and Y² are both —OH or together form the residue of catechol.

128. A compound of the formula: 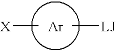
wherein
Ar is a ring or ring system, which may be at least partially aromatic but can be non-aromatic and may be substituted by one or more moieties in addition to —X and -LJ;
X is —BY¹Y², wherein Y¹ and Y² are independently selected from the group consisting of —OR¹, —SR¹, halogen or —NR¹R²,
or Y¹ and Y² taken together with the boron atom form a cyclic boron ester wherein Y¹ and Y² together form the residue of a compound having two hydroxy groups, a cyclic boron amide wherein Y¹ and Y² together form the residue of a compound having two amino groups, or a cyclic boron amide-ester
wherein Y¹ and Y² together form the residue of a compound having a hydroxy group and an amino group, wherein R¹ and R² are independently selected from the group consisting of hydrogen and a moiety in which the non-hydrogen atoms are selected from the group consisting of C, N, O and S and number from 1 to 20 which comprises at least one hydrocarbyl group which may be aliphatic or carbocyclic and optionally 1, 2 or 3 heteroatoms selected from O, N and S;
L is of the formula —(CR⁵R⁶)₁—S—(CHR⁷)ₙ—,
wherein R⁵, R⁶ and R⁷ are independently selected from the group consisting of is —H, —OH, —NH₂, —SH, —NCCH₂CO₂(C₁-C₁₄)alkyl and a moiety in which the atoms other than hydrogen and halogen are selected from the group consisting of C, N, O and S and number 1 to 20 and which comprises (i) at least one hydrocarbyl group, which is optionally substituted by halogen, and (ii) 0, 1, 2 or 3 heteroatoms selected from O, N and S;
wherein l is an integer from 0 to 6;
wherein n is an integer from 0 to 6; and
J is selected from the group consisting of
(i) -GNR³R⁴,
(ii) -GNR¹OH,
(iii) -GNR¹C(NR¹)H,
(iv) -GNR¹C(NR¹)NR¹OH,
(v) -GNR¹C(NR¹)NR¹CN,
(vi) -GNR¹C(NR¹)NR¹COR¹,
(vii) -GNR¹C(NR¹)NR¹R²
(viii) -GC(NR¹)NR¹R²,
(ix) -GC(NR¹)NR¹NR¹COR¹,
(x) -GC(NR¹)NR¹C(NR¹)NR¹R²,
(xi) -GC(NR¹)NR¹COR¹,
(xii) -GNR¹C(O)R¹
(xiii) —OC(O)NR¹R²
(xiv) —OC(O)NR¹C(O)R¹,
(xv) —C(O)ONR³R⁴
(xvi) -GN(R¹)COOR¹, and
(xvii) -GN(COOR¹)C(NH₂)═NCOOR¹,
wherein G is absent or is SO₂, CO or CO(CH₂)ₚCO, wherein p is 1, 2, 3 or 4,
wherein R¹ and R² are as defined in claim 1 or claim 75 and each R¹ group of a compound having a plurality of R¹ groups is selected from the R¹ options independently of the other R¹ group(s) of the compound,
wherein R³ and R⁴ are independently selected from hydrogen, a moiety in which the atoms other than hydrogen and halogen are selected from the group consisting of C, N, O and S and number from 1 to 20 and which comprises at least one hydrocarbyl group which is optionally substituted by halogen and may be aliphatic or carbocyclic and optionally 1, 2 or 3 heteroatoms selected from O, N and S, or R³ and R⁴ together with their attached N form a ring, which is optionally part of a fused ring system and/or substituted by C₁-C₁₀ alkyl, a C₁-C₁₀ alkyl moiety substituted by carboxyl, alkoxycarbonyl wherein the alkoxycarbonylalkyl group contains from 1 to 12 carbon atoms, alkoxy wherein the alkoxylalkyl group contains from 1 to 12 carbon atoms, hydroxy or halogen; (C₁-C₄)alkyl-(C₅-C₁₀)cyclohydrocarbyl; C₅-C₁₀ cyclohydrocarbyl, which is optionally substituted by 1, 2 or 3 moieties selected from the group consisting of 5- and 6-membered rings, hydroxy, amino, C₁, C₂, C₃ or C₄ alkyl, C₁, C₂, C₃ or C₄ alkoxy, thiol, C₁, C₂, C₃ or C₄ alkylthio, amino, nitrile, carboxy, —CHO, —C(O)alkyl, —SO₂, substituted amino and halogen, the aforesaid organic substituents optionally being substituted by halogen and the aforesaid alkyl-, cyclohydrocarbyl- and ring-containing substituents optionally being terminated by an ether or thioether linkage to the moiety which they substitute and/or, in the case of substituents containing at least one alkylic carbon atom, interrupted at an alkylic carbon by an ether or thioether linkage; or, -L-J is of the formula —C═N—NR¹—C(NR¹)NR¹R²,
wherein the compound additionally comprises all variant forms of these compounds, for example tautomers; salts, addition salts, esters, acids and their tautomers; species which are capable of existing in equilibrium with any of the aforesaid; and species which, upon administration, are capable of providing directly or indirectly any of the aforesaid.
